(19) United States
(12) Patent Application Publication
(10) Pub. No.: US 2008/0248511 A1
(43) Pub. Date: Oct. 9, 2008

(54) METHODS TO QUENCH LIGHT FROM OPTICAL REACTIONS

(75) Inventors: William Daily, Sant Maria, CA (US); Erika Hawkins, Pembal (CA); Dieter Klaubert, Arroyo Grande, CA (US); Mark McDougall, Arroyo Grande, CA (US); James Unch, Arroyo Grande, CA (US); Keith V. Wood, Mt. Horeb, WI (US); Wenhui Zhou, Santa Maria, CA (US); Ji Zhu, Ossining, NY (US)

Correspondence Address: SCHWEGMAN, LUNDBERG & WOESSNER, P.A., P.O. BOX 2938, MINNEAPOLIS, MN, 55402, US

(73) Assignee: Promega Corporation

(21) Appl. No.: 12/056,073

(22) Filed: Mar. 26, 2008

Related U.S. Application Data
(60) Provisional application No. 60/920,050, filed on Mar. 26, 2007.

(51) Int. Cl. C07D 209/16; C12Q 1/66

(52) U.S. Cl. 435/8; 548/503

(57) ABSTRACT

The present invention relates to single and dual reporter luminescence assays utilizing reagents to quench an optical, e.g., an enzyme-mediated luminescence, reaction. In one embodiment of the invention, a reagent is added to an assay which selectively quenches a first enzyme-mediated luminescence reaction without affecting a subsequent distinct enzyme-mediated luminescent reaction(s). An assay kit containing one or more selective quench reagents, and compositions comprising the quench reagent(s), are also provided.

### CROSS REFERENCE TO RELATED APPLICATIONS

This application claims the benefit of the filing date of U.S. Provisional Application Ser. No. 60/920,050, filed on Mar. 26, 2007, the disclosure of which is incorporated by reference herein.

### FIELD OF THE INVENTION

The present invention relates to enzyme-mediated single and dual optical reporter assays, and reagents that quench one or more optical reactions. For example, the present invention relates to luminescence assays utilizing at least one enzyme, and one or more luminescence quench reagents.

### BACKGROUND

Luminescence is produced in certain organisms as a result of a luciferase-mediated oxidation reaction. Luciferase genes from a wide variety of vastly different species, particularly the luciferase genes of Photinus pyralis (the common firefly of North America), Pyrophorus plagiophthalamus (the Jamaican click beetle), Renilla reniformis (the sea pansy), and several bacteria (e.g., Xenorhabdus luminescens and Vibrio spp.), are extremely popular luminescence reporter genes. Firefly luciferase is also a popular reporter for ATP concentrations, and, in that role, is widely used to detect biomass. Luminescence is also produced by other enzymes when those enzymes are mixed with certain synthetic substrates, for instance, alkaline phosphatase and adamantyl dioxetanes, or horseradish peroxidase and luminol.

Luciferase genes are widely used as genetic reporters due to the non-radioactive nature, sensitivity, and extreme linear range of luminescence assays. For instance, as few as 10-20 moles of firefly luciferase can be detected. Consequently, luciferase assays of gene activity are used in virtually every experimental biological system, including both prokaryotic and eukaryotic cell cultures, transgenic plants and animals, and cell-free expression systems. Similarly, luciferase assays of ATP are highly sensitive, enabling detection to below 10⁻¹⁶ moles.

Luciferases generate light via the oxidation of enzyme-specific substrates, called luciferins. For firefly luciferase and all other beetle luciferases, light generation occurs in the presence of magnesium ions, oxygen, and ATP. For anthozoan luciferases, including Renilla luciferase, only oxygen is required along with the luciferin. Generally, in luminescence assays of genetic activity, reaction substrates and other luminescence activating reagents are introduced into a biological system suspected of expressing a reporter enzyme. Resultant luminescence, if any, is then measured using a luminometer or any suitable radiant energy-measuring device. The assay is very rapid and sensitive, and provides gene expression data quickly and easily, without the need for radioactive reagents. Reporter assays other than for genetic activity are performed analogously.

The conventional assay of genetic activity using firefly luciferase has been further improved by including coenzyme A (CoA) in the assay reagent to yield greater enzyme turnover and thus greater luminescence intensity (U.S. Pat. No. 5,283,179). Using this reagent, luciferase activity can be readily measured in luminometers or scintillation counters. The luciferase reaction, modified by the addition of CoA to produce persistent light emission, provides an extremely sensitive and rapid assay for quantifying luciferase expression in genetically altered cells or tissues.

Light refracted from one luminous sample may interfere with the subsequent measurement of signal from luminescent samples in successive wells in clear multi-wells. Moreover, with respect to the cumulative nature of refracted light emanating from multiple luminous samples within a single clear plastic plate, while the luminescent signal in the first sample well could be measured accurately, sequential activation of luminescent reactions in following wells would lead to increasingly inaccurate measurements due to the cumulative emission of photons refracted through the plastic from all of the previous samples. This problem of refracted light, or “refractive cross-talk”, would be further exacerbated when brightly illuminated wells were situated adjacent to negative control wells in which no luminescence was generated, or when brightly lit wells were situated near relatively dim wells. This makes determining the absolute and baseline luminescence in a clear multi-well plate quite difficult.

Opaque plates formed of white plastic can yield greater luminescence sensitivity than clear plates, however, photons are readily scattered from adjacent wells, again introducing cross-talk interference between wells. Here, the cross-talk is referred to as “reflective cross-talk.” Moreover, black 96-well plates, originally intended for fluorescent applications, are not ideal for luminescence applications because the sample signal is greatly diminished due to the non-reflective nature of the plastic. Further, opaque plates are inferior for cultured cells because cultured cells cannot be viewed or photographed through the opaque plate, and the plates have undetermined effects on cell adhesion and growth characteristics of the cells.

Luciferases are one of a number of reporters, e.g., firefly luciferase, Renilla luciferase, chloramphenicol acetyl transferase (CAT), beta-galactosidase (lacZ), beta-glucuronidase (GUS) and various phosphatases, such as secreted alkaline phosphatase (SEAP) and uteroferrin (Uf; an acid phosphatase), that have been combined and used as co-reporters of genetic activity. A dual enzyme reporter system relates to the simultaneous use, expression, and measurement of two individual reporter enzymes within a single system. In genetic reporting, dual reporter assays are particularly useful for assays in individual cells or cell populations (such as cells dispersed in culture, segregated tissues, or whole animals) genetically manipulated to simultaneously express two different reporter genes. Most frequently, the activity of one gene reports the impact of the specific experimental conditions, while the activity of the second reporter gene provides an internal control by which all sets of experimental values can be normalized. Dual enzyme reporter technology can also be employed with cell-free reconstituted systems such as cellular lysates derived for the simultaneous translation, or coupled transcription and translation, of independent genetic materials encoding experimental and control reporter enzymes. Immunoassays may, likewise, be designed for dual reporting of both experimental and control values from within a single sample.

The performance of any dual enzyme reporter assay is limited by the characteristics of the constituent enzyme chemistries and the ability to correlate their respective resulting data sets. Disparate enzyme kinetics, assay chemistries and incubation requirements of various reporter enzymes can complicate combining two reporter enzymes into an integrated, single tube or well dual reporter assay format.

What is needed is the identification of luminescence quench agents for use in a method to assay an enzyme-mediated luminescence reaction or a series of enzyme-mediated luminescence reactions.

### SUMMARY OF THE INVENTION

The present invention is directed to compositions and methods to quench (reduce, inhibit or eliminate) light generated by one luminescent reporter so that a second luminescent reporter signal may be subsequently measured. Such a method provides for multiplexing various combinations of light producing reactions with great flexibility. Thus, the invention includes compositions and methods for luminescence assays which utilize one or more reagents to rapidly and efficiently quench, e.g., selectively quench, a first enzyme-mediated luminescence reaction, e.g., an anthozoan, copepod, or decapod luciferase-mediated luminescence reaction. Also included are compositions and methods for luminescence assays which utilize coelenterazine or a derivative thereof as a substrate in an enzyme-mediated luminescence reaction. Selective reagents such as quenching reagents for use in the methods and compositions of the invention include, but are not limited to, a compound of formula (I):

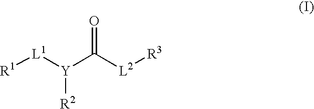

wherein

Y is N or O;

L¹ is (C₁-C₆)alkylene or a direct bond;

R¹ is alkyl, aryl, heteroaryl, or heterocycle;

R² is H, (C₁-C₆)alkyl, or absent;

or L¹ is a direct bond and R¹ and R² together with the nitrogen attached to R² form a heteroaryl or heterocycle group;

L² is optionally unsaturated straight chain or branched (C₁-C₆)alkylene or (C₁-C₆)alkylene-O—;

R³ is alkyl, aryl, heteroaryl, heterocycle;

wherein any alkyl, alkylene, aryl, heteroaryl, or heterocycle is optionally substituted with one to five alkyl, alkenyl, alkoxy, alkanoyl, alkanoyloxy, alkoxycarbonyl, cycloalkyl, cyano, nitro, halo, hydroxy, mercapto, oxo, —SOₙR⁴, N(R^{x})(R^{Y}), N(R^{x})(R^{Y})alkyl, N⁺(R^{X})(R^{Y})(R^{Z}), or N⁺(R^{X})(R^{Y})(R^{Z})alkyl groups wherein R^{X}, R^{Y}, and R^{Z} are each independently H, alkyl, aryl, heteroaryl, or heterocycle; and each substituent is optionally substituted with one to three R³ groups; and a nitrogen atom of a nitrogen heterocycle is optionally protected with a nitrogen protecting group;

n is 0, 1, 2, or 3; and

R⁴ is H, alkyl, or aryl;

or a salt thereof,

or a compound of formula (IV):

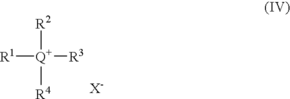

wherein

Q is N or P;

R¹ and R² are each independently alkyl, cycloalkyl, aryl, heteroaryl, heterocycle, arylalkyl;

R³ and R⁴ are each independently (C₁-C₆)alkyl; and

X is an organic or inorganic counterion.

In one embodiment, the selective quenching reagents are inhibitors of anthozoan, copepod, or decapod luciferases. In one embodiment, one or more of the selective quenching reagents are in compositions optionally in combination with other selective quenching reagents, such as a substrate analog inhibitor, e.g., an inhibitor that is structurally similar to a native substrate for the enzyme (i.e., a substrate for the enzyme which occurs in nature) and inhibits the enzyme and/or one that competes with a light generating substrate for the active site on an enzyme (a competitive inhibitor); a sequestering agent, e.g., an agent which physically separates a substrate for an anthozoan, copepod, or decapod luciferase, e.g., colenterazine or a derivative thereof, from the anthozoan, copepod, or decapod luciferase, for instance, the agent physically separates the first substrate or luciferase into micelles or shifts the solubility of the luciferase substrate or the luciferase, so as to inhibit an interaction between the luciferase substrate and the luciferase which results in light generation but does not substantially alter a reaction between a second, distinct enzyme and its corresponding substrate; a colored compound, which quenches the color of light emitted by an anthozoan, copepod, or decapod luciferase-mediated luminescence reaction but not all enzyme-mediated reactions.

The selective quenching agents substantially quench an anthozoan, copepod, or decapod luciferase-mediated luminescence reaction but not all enzyme-mediated luminescence reactions to the same degree. Thus, such reagents are selective in that, in an effective amount, they quench an anthozoan, copepod, or decapod luciferase-mediated luminescence reaction while permitting efficient generation and recordation of light from at least one other distinct enzyme-mediated luminescence reaction. In one embodiment, selective quenching reagents for an anthozoan, copepod, or decapod luciferase-mediated luminescence reaction are not reagents that selectively quench luminescence from a beetle luciferase-mediated reaction.

The invention provides compositions having a compound of formula (I), a compound of formula (IV), or a combination thereof, and optionally one or more other quench reagents, reducing agents, cell lysing agents, or combinations thereof. In one embodiment, the compositions are useful in luciferase-based assays, including coupled assays, such as those employing luciferin derivatives that are prosubstrates of luciferase and a substrate for another enzyme or other molecule. In one embodiment, a compound of formula (I), a compound of formula (IV), or a combination thereof, is present in a luciferase assay buffer.

A “substantial” quenching of light is a percent-quench or fold-quench equal to or greater than the quench for a reference, e.g., a first enzyme-mediated luminescence reaction. For instance, in one embodiment, a selective quench reagent may substantially quench a first enzyme-mediated luminescence reaction by 35-fold, but would not quench or quenches a second, distinct enzyme-mediated luminescence reaction by less than 35-fold, therefore, it is a selective quench reagent for the first reaction relative to the second reaction. In contrast, if a quench reagent quenches a first enzyme-mediated luminescence reaction by 35-fold and quenches a second, distinct enzyme-mediated luminescence reaction by 35-fold or more, it is not a selective quench reagent for the first reaction relative to the second reaction. In one embodiment, a selective quench reagent may substantially quench a first enzyme-mediated luminescence reaction by at least 15% relative to a corresponding enzyme-mediated reaction in the absence of the selective quench reagent and/or relative to a second, distinct enzyme-mediated luminescence reaction.

In one embodiment, a selective quench reagent may quench luminescence from a luminescent reaction by at least 15-fold, preferably by at least 25-fold, more preferably by at least 35-fold, and even more preferably by at least 50-fold, and yet even more preferably by at least 100-fold or more, e.g., 200-fold, 300-fold, 400-fold, 900-fold, 10,000 fold or more, when compared to a corresponding enzyme-mediated reaction in the absence of the selective quench reagent and/or relative to a distinct luminescent reaction. In one embodiment, a plurality of selective quench reagents are combined so as to increase the fold- or percent-quench. In one embodiment, a selective quench reagent may quench luminescence from a luminescent reaction by at least 20%, e.g., 30%, 40%, 50% or more up to at least 99% when compared to a corresponding enzyme-mediated reaction in the absence of the selective quench reagent and/or relative to a distinct luminescent reaction.

A luminescence reporter is a molecule which mediates a luminescence reaction, and by doing so, yields information about the state of a chemical or biochemical system. Examples are genetic reporters (Wood, 1995), immunoassay reporters (Bronstein et al., 1991), ATP reporters (Schram, 1991), as well as reporters of other cellular molecules such as enzymes or cofactors. Enzymes are proteins which catalyze a chemical transformation, and thus are not changed by that transformation. Because the enzyme is regenerated at the conclusion of the transformation, it is available for additional cycles of transformation; enzymes thus have the capacity for substrate turnover. This property allows the capacity for continuous luminescence in an enzyme-mediated luminescence reaction. An enzyme-mediated luminescence reaction is a chemical reaction mediated by an enzyme which yields photons as a consequence of the reaction. The enzyme in an enzyme-mediated luminescence reaction effectively enables the reaction when the majority of the luminescence generated in the reaction follows as a consequence of the action of the enzyme.

The present invention is ideally suited for luminescence reactions as photons are transient in existence. Therefore, quenching of an enzymatic reaction which produces photons immediately diminishes the product photons present in the sample. Thus, once the luminescence measurement is taken, and the enzymatic reaction is quenched, there is no build-up of product photons in the sample. In essence, luminescence reactions can be “turned off” without leaving an accumulation of the experimental or control signal (i.e., photons) within the sample. The same cannot be said of analogous enzymatic reactions in which the buildup of a stable chemical product is measured, or the slow decay of an accumulated chemical product is measured. Here, quenching enzymatic reactions leading to a chemical product still leaves a large accumulation of the chemical product within the sample, leading to potential interference with other assays being simultaneously or sequentially taken from the sample.

Examples of enzymes which mediate luminescence reactions include, but are not limited to, beetle luciferases, which all catalyze ATP-mediated oxidation of beetle luciferin; anthozoan luciferases, which all catalyze oxidation of coelenterazine (Ward, 1985); a peroxidase such as horseradish peroxidase, which catalyzes a reaction involving luminol (Thorp et al., 1986); and a phosphatase such as alkaline phosphatase, which catalyzes a reaction with adamantyl 1,2-dioxetane phosphate (Schaap et al., 1989), as well as other enzymes which catalyze a reaction with a dioxetane substrate, e.g., a substrate such as 3-(2′-spiroadamantane)-4-methoxy-4-(3″-phosphoryloxy)phenyl-1,2-dioxetane, disodium salt, or disodium 3-(4-methoxyspiro[1,2-d]oxetane-3,2′(5′-chloro)-tricyclo-[3.3.1.1^{3,7}]decan]-4-yl]phenyl phosphate, or disodium 2-chloro-5-(4-methoxyspiro{1,2-dioxetane-3,2′-(⁵′-chloro)-tricyclo {3.3.1.13,7]decan}-4-yl)-1-phenyl phosphate, disodium 2-chloro-5-(⁴-methoxyspiro {1,2-dioxetane-3,2′-tricyclo[3.3.1.13,7]decan}-4-yl)-1-phenzyl phosphate (AMPPD, CSPD, CDP-Star® and ADP-Star™, respectively), 3-(2′-spiroadamantane)-4-methoxy-4-(3″-β-D-galactopyranosyl)phenyl-1,2-dioxetane (AMPGD), 3-(4-methoxyspiro[1,2-d]oxetane-3,2′-(5′-chloro)tricyclo[3.3.1.1^{3,7}]-decan]-4-yl-phenyl-β-D-galactopyranoside (Galacton®), 5-chloro-3-(methoxyspiro[1,2-d]oxetane-3,2′-(5′-chloro)tricyclo[3.3.1^{3,7}]decan-4-yl-phenyl-β-D-galactopyranoside (Galacton-Plus®), 2-chloro-5-(4-methoxyspiro[1,2-d]oxetane-3,2′(5′-chloro)-tricyclo-[3.3.1.1^{3,7}]decan]-4-yl)phenyl β-D-galacto-pyranoside (Galacton-Star®), and sodium 3-(4-methoxyspiro{1,2-dioxetane-3,2′-(5′-chloro)-tricyclo[3.3.1.1^{3,7}]decan}-4-yl)phenyl-β-D-glucuronate (Glucuron™); or a functional equivalent of such an enzyme. A functional equivalent of a specified enzyme includes a recombinant enzyme that maintains the ability to catalyze the same luminescence reaction as the corresponding nonrecombinant wild-type enzyme, and thus it remains in the same group of enzymes, but has an altered structure relative to a corresponding wild-type enzyme.

An example of a functional equivalent of an enzyme is a genetic fusion of one enzyme to another peptide or protein to yield a bifunctional hybrid protein (Kobatake et al., 1993). Another example of a functional equivalent of a specified enzyme includes a recombinant enzyme that maintains the ability to catalyze the same luminescence reaction as the corresponding nonrecombinant wild-type enzyme but has one or more amino acid substitutions relative to the corresponding nonrecombinant wild-type enzyme, e.g., up to about 50%, or fewer, e.g., about 20%, or about 10%, of the residues are substituted.

Luciferases can be isolated or obtained from a variety of luminous organisms, such as the firefly luciferase of Photinus pyralis or the Renilla luciferase of Renilla reniformis. A “luciferase” as used herein shall mean any type of luciferase originating from any natural, synthetic, or genetically-altered source, including, but not limited to: luciferases isolated from the firefly Photinus pyralis or other beetle luciferases (such as luciferases obtained from click beetles (e.g., Pyrophorus plagiophthalamus) or glow worms (Pheogodidae spp.)), the sea pansy Renilla reniformis, Vargula species, e.g., Vargula hilgendorfii, Gaussia species, Oplophorus species, the limpet Latia neritoides, and bacterial luciferases isolated from such organisms as Xenorhabdus luminescens, and Vibrio fisherii; and functional equivalents thereof.

In one embodiment, the present invention relates to luminescence assays which employ one or more reagents which quench an enzyme-mediated luminescence reaction. In one embodiment, the one or more quench reagent(s) are added in an amount effective to quench luminescence by at least 15-fold, preferably by at least 25-fold, more preferably by at least 35-fold, and even more preferably by at least 50-fold, and yet even more preferably by at least 100-fold or more, e.g., 200-fold, 300-fold, 400-fold, 900-fold, or 10,000 fold relative to the luminescence generated in the absence of the reagent(s). In one embodiment, one or more quench reagent(s) are added in an amount effective to quench luminescence by 15% or more, e.g., 20%, 30%, 40%, 50% up to at least 99%, or more. Preferably, the quench reagent is a selective quench reagent as described herein.

As described hereinbelow, hydrophobic quaternary ammonium and phosphium molecules, e.g., a compound of formula (IV), and heterocycles such as indole containing molecules, e.g., a compound of fomula (I), quenched Renilla luciferase without similarly quenching firefly luciferase. For a compound of formula (I), the Renilla luciferase luminescence was substantially decreased (e.g., 20 to 100%) in a concentration-dependent manner with much less, if any, effect on firefly luciferase luminescence. As also described herein, a subset of compounds at high concentrations (for example, about 3 mM) can inhibit Renilla luciferase by up to approximately 500-fold with little impact on other enzymes like firefly luciferase. In contrast, other inhibitors affected Renilla luciferase by only about 10-fold before inhibiting firefly luciferase.

For example, the invention includes a method of assaying an enzyme-mediated luminescence reaction. The method includes detecting or determining luminescence energy produced by an anthozoan luciferase-mediated luminescence reaction and quenching photon emission from the anthozoan luciferase-mediated luminescence reaction by introducing a composition comprising at least one selective quench reagent to the luminescence reaction. In another embodiment, the method includes detecting or determining luminescence energy produced by an anthozoan luciferase-mediated luminescence reaction and quenching photon emission from the first enzyme-mediated luminescence reaction by introducing a composition comprising at least two selective quench reagents to the luminescence reaction.

In another embodiment, the present invention relates to luminescence assays which employ one or more reagents which selectively quench an anthozoan luciferase-mediated luminescence reaction without substantially quenching the light generated by a second distinct, sequential enzyme-mediated luminescence reaction. In one embodiment, at least one reagent for the second distinct, enzyme-mediated luminescence reaction is present in the anthozoan luciferase-mediated luminescence reaction.

In one embodiment of the invention, an anthozoan luciferase-mediated luminescence reaction is first initiated by addition of an appropriate initiating reagent or reagents to a sample to yield a reaction mixture. The luminescence signal produced in the reaction mixture is then measured, e.g., so as to detect the presence or amount of one or more molecules in the sample. One or more selective quench reagents are then added so as to diminish the luminescence signal within a relatively short time interval after introduction of the selective quench reagent. In one embodiment, the one or more selective quench reagent(s) are added in an amount effective to quench luminescence by at least 15-fold, preferably by at least 25-fold, more preferably by at least 35-fold, and even more preferably by at least 50-fold, and yet even more preferably by at least 100-fold or more, e.g., 200-fold, 300-fold, 400-fold, or 900-fold, relative to the luminescence generated in the absence of the reagent(s).

By extinguishing the luminescence signal from the enzyme in the sample, addition of the selective quench reagent(s) prevents light from previously-activated samples from interfering with light measurements in subsequently-activated samples, e.g., in a multisample assay format. The second luminescence signal produced is then measured. Preferably, the presence or amount of two or more molecules are detected in a single reaction, e.g., all reactions are conducted in a single receptacle, e.g., well.

The sample employed in the methods of the invention may be a cell lysate, an in vitro transcription/translation reaction, a supernatant of a cell culture, a physiological fluid sample, e.g., a blood, plasma, serum, cerebrospinal fluid, tears or urine sample, and may include intact cells. The cells, cell lysate, or supernatant may be obtained from prokaryotic cells or eukaryotic cells, and the physiological fluid from any avian, reptile, amphibian or mammal. The initiating reagent or reagents may thus be added to intact cells, cell lysates, or supernatants or physiological fluids. The quench reagent may also be added to intact cells, or to a cell lysate, an in vitro transcription/translation reaction, or a physiological fluid sample or supernatant sample.

The present invention thus includes dual luminescence assays which employ one or more reagents which selectively quench an anthozoan luciferase-mediated luminescence reaction without quenching another distinct enzyme-mediated luminescence reaction, i.e., the two distinct enzymes respond differently to various reagents, thereby allowing one of the enzyme-mediated luminescence reactions to be selectively quenched. In one embodiment, both reactions are luciferase-mediated reactions, e.g., the first luciferase-mediated luminescence reaction is a Renilla luciferase-mediated luminescence reaction, which is selectively quenched while allowing a second distinct luciferase-mediated luminescence reaction, for instance, a firefly luciferase-mediated luminescence reaction, to proceed without substantially quenching the luminescence from the second reaction. For example, Renilla luciferase can be selectively quenched using reagents which are selective for anthozoan luciferases and have substantially no effect on other reporters present in or reactions occurring in the sample.

Exemplary reagents for selectively quenching anthozoan luciferase-mediated luminescence reactions, as well as other luminescence reactions, include, but are not limited to, a compound of formula (I), a compound of formula (IV), or a combination thereof, optionally in combination with a substrate analog inhibitor which is structurally related to coelenterazine, a detergent, e.g., one which sequesters an anthozoan luciferase substrate but not the anthozoan luciferase enzyme in micelles, and/or a colored compound which selectively quenches the color emitted by the first reaction, for instance, for blue light, a selective quench reagent is a yellow colored compound.

The quench reagent for the first reaction and the activation reagent for the second reaction can be added simultaneously or sequentially. When the quench reagent is formulated to allow simultaneous initiation of a second enzyme-mediated luminescence reaction, the reagent is referred to as a “quench-and-activate” reagent. Hence, a quench-and-activate reagent simultaneously quenches the anthozoan luciferase-mediated reaction and initiates the second enzymatic reaction and such an assay thus allows the sequential measurement of two separate and distinct luminescence reporters within one sample. As a result, one of the luminescence reporters can be used as an internal standard, while the other is used to report the impact of the experimental variables. Alternatively, each reporter can report two different variables, e.g., the presence of a particular protease and ATP concentration, in a sample. This strategy greatly expedites multiplexing to provide quick, automatable, accurate, and reproducible results using standard multi-well plates and instrumentation.

For instance, the luminescence chemistries of beta-galactosidase, beta-glucuronidase, horseradish peroxidase, alkaline phosphatase or luciferases can be utilized in a dual reporter luminescence assay with a distinct luminescence enzyme. In one embodiment, one of the two luminescent enzymes acts as an internal standard, while the other functions as an experimental marker for gene activity or the presence or amount of an enzyme, substrate or cofactor for an enzyme-mediated reaction. Moreover, the present invention is particularly useful for high-throughput automated assays based on enzyme-mediated luminescence reporter systems, using conventional transparent or opaque multi-well plates.

In one embodiment, the invention includes a method of assaying an enzyme-mediated luminescence reaction. The method includes detecting or determining luminescence energy produced by an anthozoan luciferase-mediated luminescence reaction, and quenching photon emission from the anthozoan luciferase-mediated luminescence reaction and/or quenching the anthozoan-mediated luminescence reaction by introducing at least one quench reagent to the luminescence reaction. In one embodiment, the quench reagent is a compound of formula (I), a compound of formula (IV), or a combination thereof. In one embodiment, an anthozoan luciferase-mediated luminescence reaction may be employed to detect the presence or amount of a molecule, e.g., a protease, which reaction is quenched prior to initiating a beetle luciferase-mediated luminescence reaction, e.g., to detect ATP concentration. Accordingly, the present invention allows multiplexing of enzyme-mediated assays for one or more enzymes, one or more substrates and/or one or more cofactors, or any combination thereof.

The invention thus provides a method for measuring the activity or presence of at least one molecule in a sample. The method includes providing a sample that may contain at least one molecule for an enzyme-mediated reaction, e.g., the sample may contain the enzyme, and contacting the sample with a reaction mixture for the enzyme-mediated reaction which lacks the molecule. For example, the reaction mixture contains a substrate for the enzyme to be detected, e.g., a modified substrate for an enzyme, such as a coelenterazine modified with a protease substrate, and the anthozoan luciferase, where the presence or amount of the molecule in the sample is capable of being detected by an enzyme-mediated luminescence reaction. In one embodiment, after or concurrently with quenching the anthozoan luciferase-mediated luminescence reaction, the reaction mixture is contacted with reagents to detect a molecule capable of being detected by another enzyme-mediated luminescence reaction.

The methods of the present invention allow the detection of multiple enzymes, substrates or cofactors in a sample, e.g., a sample which includes eukaryotic cells, e.g., yeast, avian, plant, insect or mammalian cells, including but not limited to human, simian, murine, canine, bovine, equine, feline, ovine, caprine or swine cells, or prokaryotic cells, or cells from two or more different organisms, or cell lysates or supernatants thereof. The cells may not have been genetically modified via recombinant techniques (nonrecombinant cells), or may be recombinant cells which are transiently transfected with recombinant DNA and/or the genome of which is stably augmented with a recombinant DNA, or which genome has been modified to disrupt a gene, e.g., disrupt a promoter, intron or open reading frame, or replace one DNA fragment with another. The recombinant DNA or replacement DNA fragment may encode a molecule to be detected by the methods of the invention, a moiety which alters the level or activity of the molecule to be detected, and/or a gene product unrelated to the molecule or moiety that alters the level or activity of the molecule.

In one embodiment, the present invention relates to a method of measuring the presence or amount of multiple enzymes in a single aliquot of cells or a lysate thereof. For enzymes present in different cellular locations, such as a secreted and an intracellular enzyme, a substrate for one of the enzymes can be added to a well with intact cells. Thus, in one embodiment, the presence or amount of the secreted enzyme is detected by contacting intact cells with reagents for an anthozoan luciferase-mediated luminescence reaction and a substrate for the secreted enzyme, which substrate, when cleaved, yields a substrate for the luminescence reaction. A selective quench reagent is added concurrently with, before or after cells are lysed, and the presence or amount of the intracellular enzyme is detected, e.g., where the detection of the intracellular enzyme is in the same receptacle, for instance, same well, as that for the secreted enzyme. Detection of the anthozoan luciferase-mediated reaction may be before cell lysis or after cell lysis but before quenching. Thus, the present methods can be employed to detect any molecule in an enzyme-mediated reaction including any enzyme, substrate or cofactor, or any set thereof. Enzymes employed in the methods, either enzymes to be detected or enzymes which are useful to detect a substrate or cofactor, can be selected from any combination of enzymes including recombinant and endogenous (native) enzymes.

The invention also includes quench reagents, compositions and assay kits for analyzing samples using enzyme-mediated luminescence reactions. For example, the invention includes an enzyme-mediated luminescence reaction assay kit which includes at least one functional enzyme substrate corresponding to the anthozoan luciferase-mediated luminescence reaction to be assayed or the anthozoan luciferase; a suitable first container, the at least one functional enzyme substrate or the anthozoan luciferase disposed therein; a composition comprising at least one selective quench reagent, wherein the selective quench reagent comprises a compound of formula (I) or (IV), or a combination thereof, a suitable second container, the composition disposed therein; and optionally instructions for use. The functional enzyme substrates may be obtained from organisms (“native” substrates) or prepared in vitro (“synthetic” substrates). In another embodiment, the enzyme-mediated luminescence reaction assay kit includes at least one functional enzyme substrate for a beetle luciferase-mediated luminescence reaction to be assayed or the beetle luciferase; a suitable first container, the at least one functional enzyme substrate or the beetle luciferase disposed therein; a composition comprising at least one of a compound of formula (I) or (IV), or a combination thereof; a suitable second container, the composition disposed therein; and optionally instructions for use. Kits may also include other reagents, e.g., other functional enzymes or substrates.

In another embodiment, the invention includes a dual reporter enzyme-mediated luminescence reaction assay kit which includes a first functional enzyme substrate for a corresponding anthozoan luciferase-mediated luminescence reaction being assayed; a suitable first container, the first functional enzyme substrate disposed therein; a quench-and-activate composition which includes at least one selective quench reagent for an anthozoan luciferase which includes a compound of formula (I) or (IV), or a combination thereof and a second and distinct functional enzyme substrate corresponding to a second and distinct enzyme-mediated luminescence reaction; a suitable second container, the quench-and-activate composition disposed therein; and optionally instructions for use. In yet another embodiment, the dual reporter enzyme-mediated luminescence reaction assay kit includes a first functional enzyme substrate for a corresponding anthozoan luciferase-mediated luminescence reaction being assayed; a suitable first container, the first functional enzyme substrate disposed therein; a quench-and-activate composition comprising at least two selective quench reagents which includes a compound of formula (I) or (IV) and a second and distinct functional enzyme substrate corresponding to a second and distinct enzyme-mediated luminescence reaction; a suitable second container, the quench-and-activate composition disposed therein; and optionally instructions for use.

Accordingly, the invention also provides compounds of formula (I):

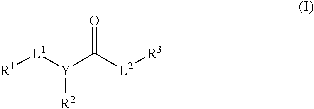

wherein

Y is N or O;

L¹ is (C₁-C₆)alkylene or a direct bond;

R¹ is alkyl, aryl, heteroaryl, or heterocycle;

R² is H, (C₁-C₆)alkyl, or absent;

or L¹ is a direct bond and R¹ and R² together with the nitrogen attached to R² form a heteroaryl or heterocycle group;

L² is optionally unsaturated straight chain or branched (C₁-C₆)alkylene or (C₁-C₆)alkylene-O—;

R³ is alkyl, aryl, heteroaryl, heterocycle;

wherein any alkyl, alkylene, aryl, heteroaryl, or heterocycle is optionally substituted with one to five alkyl, alkenyl, alkoxy, alkanoyl, alkanoyloxy, alkoxycarbonyl, cycloalkyl, cyano, nitro, halo, hydroxy, mercapto, oxo, —SOₙR⁴, N(R^{x})(R^{Y}), N(R^{x})(R^{Y})alkyl, N⁺(R^{X})(R^{Y})(R^{Z}), or N⁺(R^{X})(R^{Y})(R^{Z})alkyl groups wherein R^{X}, R^{Y}, and R^{Z} are each independently H, alkyl, aryl, heteroaryl, or heterocycle; and each substituent is optionally substituted with one to three R³ groups; and a nitrogen atom of a nitrogen heterocycle is optionally substituted with an optionally substituted alkyl or acyl group, or with a nitrogen protecting group;

n is 0, 1, 2, or 3; and

R⁴ is H, alkyl, or aryl;

or a salt thereof;

provided that the compound of formula (I) is not 3-(3-(2-(dimethylamino)ethyl)-1H-indol-5-yl)-N-(4-methoxybenzyl)acrylamide; and compounds of formula (IV):

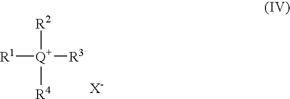

wherein

Q is N or P;

R¹ and R² are each independently alkyl, cycloalkyl, aryl, heteroaryl, heterocycle, arylalkyl;

R³ and R⁴ are each independently (C₁-C₆)alkyl; and

X is an organic or inorganic counterion; their methods of synthesis, and their methods of use.

### BRIEF DESCRIPTION OF THE FIGURES

FIGS. 1A-C show remaining Renilla luciferase luminescence in the presence of various compounds of formula (I), according to various embodiments.

FIGS. 2A-C illustrate various compounds of formula (I), according to various embodiments.

FIG. 3 illustrates compounds of formula (I), according to various embodiments.

FIG. 4 illustrates Renilla luciferase inhibition by four compounds, according to various embodiments.

FIG. 5 shows certain compounds of the invention (3077, 3078, 1424 and 1425), according to various embodiments.

FIG. 6 illustrates coelenterazines useful in the methods of the invention.

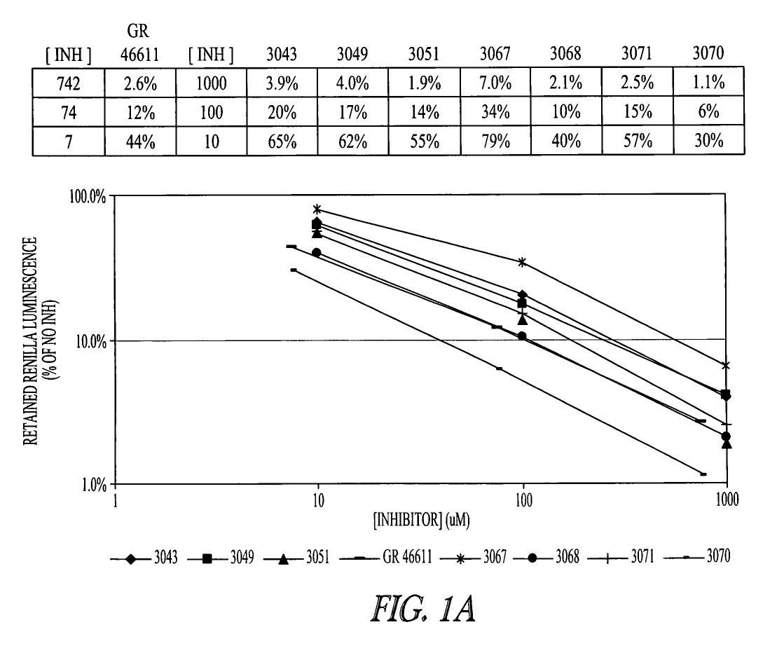

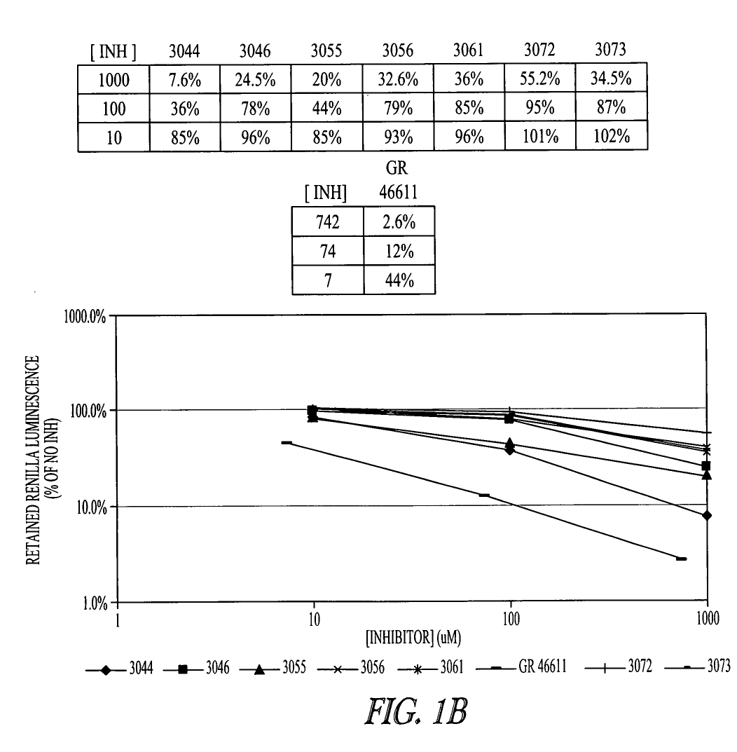

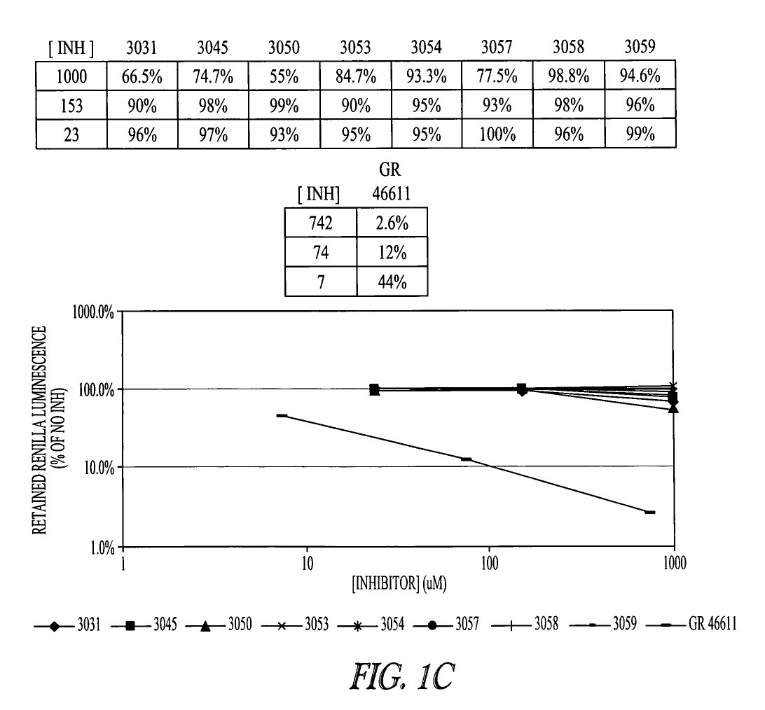

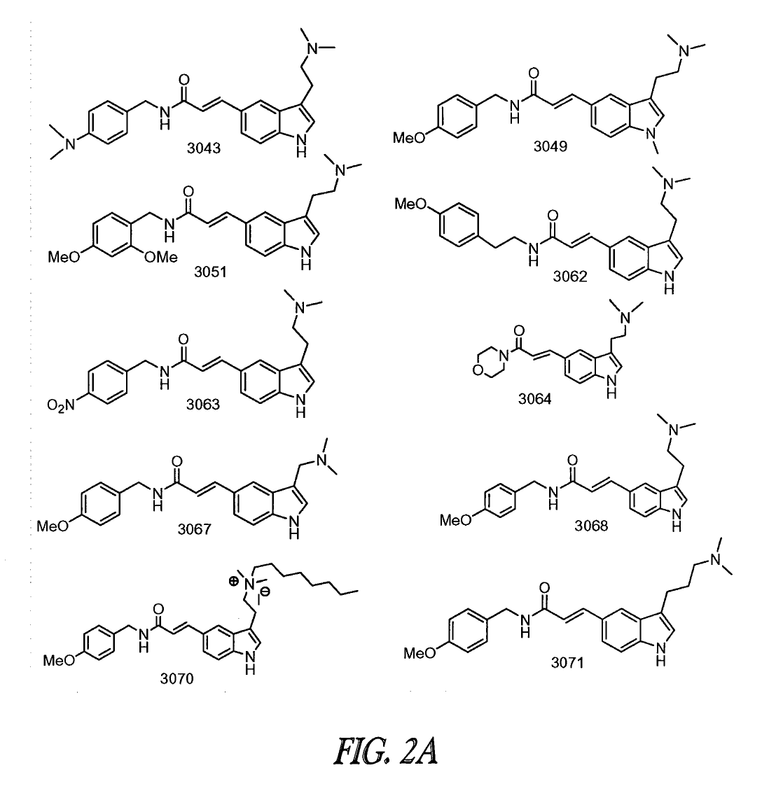

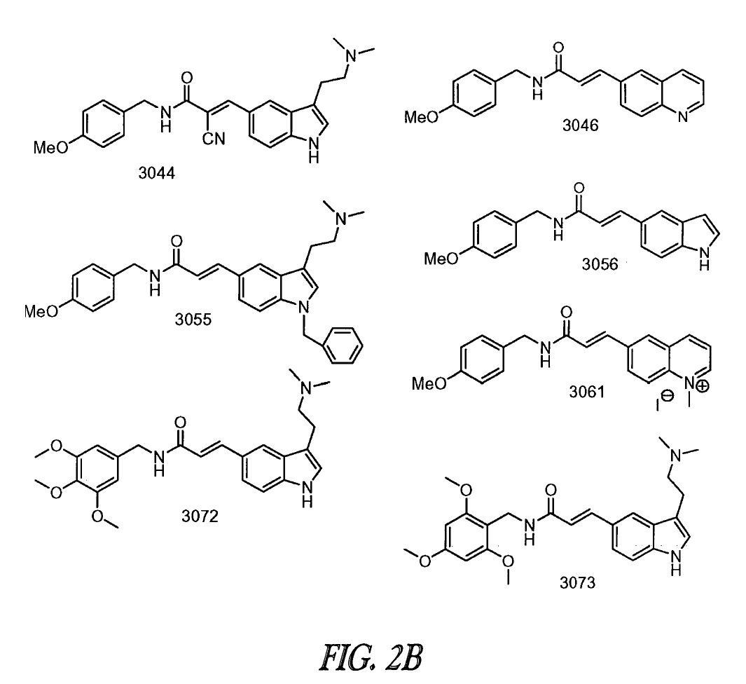

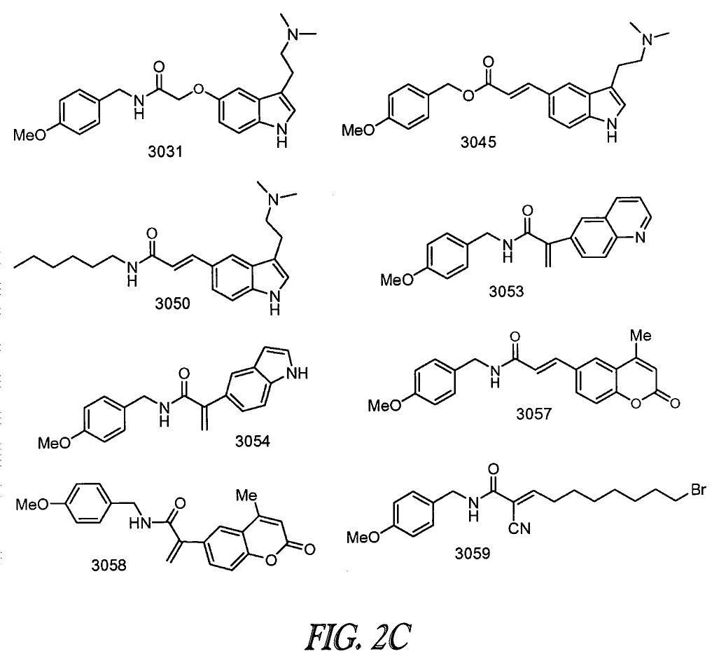

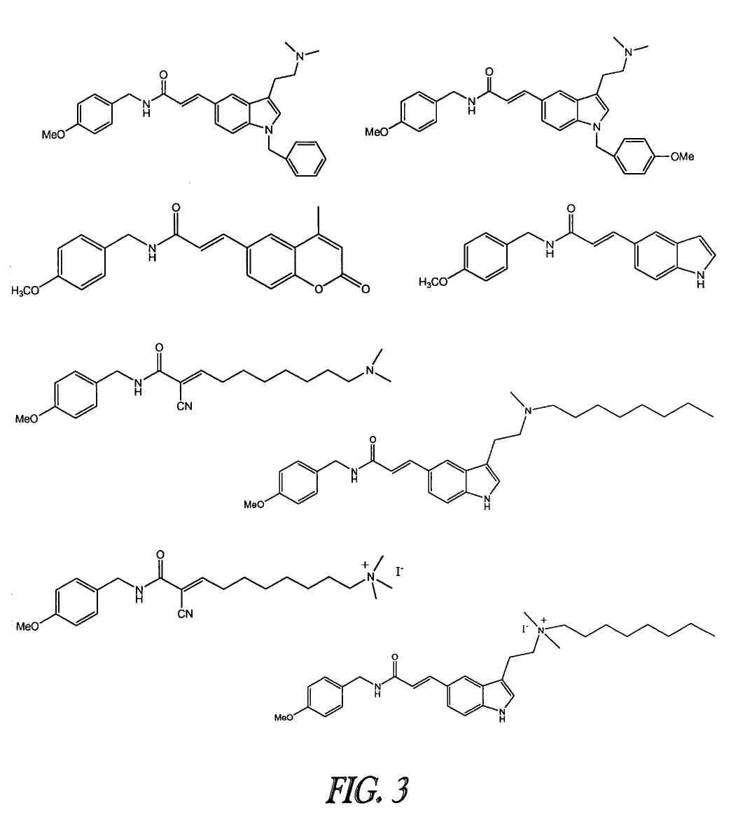

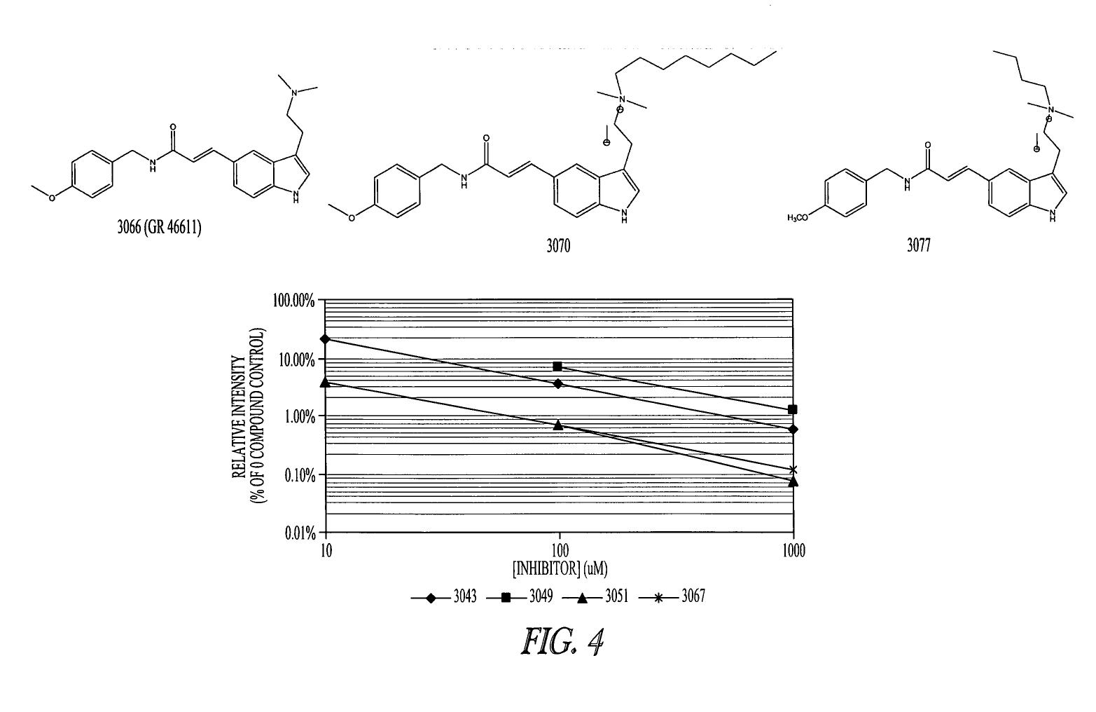

### DETAILED DESCRIPTION

The present invention includes a method of assaying enzyme-mediated luminescence reactions. In one embodiment, the method includes initiating an anthozoan luciferase-mediated luminescence reaction, quantifying luminescence energy produced by the luminescence reaction, and quenching photon emission from the anthozoan luciferase-mediated luminescence reaction by introducing a composition comprising at least one quench reagent to the luminescence reaction. The quench reagent is a selective quench reagent, i.e., the quench reagent does not quench all luminescence reactions and so a second sequential enzyme-mediated luminescence reaction may be conducted. Thus, the invention provides compositions and methods useful to quench as well as selectively quench a first enzyme-mediated luminescence reaction.

The present invention also includes a dual reporter method for assaying enzyme-mediated luminescence reactions in which an anthozoan luciferase-mediated luminescence reaction is initiated, and the luminescent energy of the first reaction detected or determined. This is followed by introduction of a composition comprising at least one selective quench reagent, i.e., a quench reagent which quenches at least the anthozoan luciferase-mediated reaction but not all luminescence reactions, then by a composition comprising a mixture capable of activating or initiating the second enzyme-mediated luminescence reaction, or by a quench-and-activate composition capable of selectively quenching the anthozoan luciferase-mediated luminescence reaction and simultaneously initiating a second enzyme-mediated luminescence reaction which is distinct from the anthozoan luciferase-mediated luminescence reaction.

The luminescent energy produced by the second enzyme-mediated luminescence reaction is then detected or determined. Optionally, the second enzyme-mediated luminescence reaction may subsequently be quenched by the addition of a second quench reagent, which may be selective for the second enzyme-mediated luminescence reaction and preferably does not quench or does not substantially quench a third enzyme-mediated luminescence reaction.

The selective quench reagents are suited for use with automatic injectors and in microtiter plates (both opaque and clear) such as conventional 96-well plates. Because the selective quench reagent effectively reduces or extinguishes the luminescence signal from within a sample, multiple luminescence assays can be performed within a clear multi-well plate without refractive cross-talk between samples.

In one embodiment, at least one of the enzyme-mediated luminescence reactions is an anthozoan luciferase-mediated reaction. Among luciferases specifically, the method of the present invention may be used to assay luminescence reactions mediated by anthozoan luciferases including Renilla reniformis luciferase, as well as beetle luciferases, including Photinus pyralis luciferase, and Pyrophorus plagiophthalamus luciferase. In one embodiment, one luciferase-mediated luminescence reaction is mediated by Renilla luciferase and the second enzyme-mediated reaction is mediated by a distinct enzyme such as beetle luciferase, horseradish peroxidase, alkaline phosphatase, beta-glucuronidase or beta-galactosidase.

A quench reagent for a particular enzyme is likely to quench enzymes in the same class. Thus, generally a quench reagent for Renilla luciferase is likely to quench other anthozoan luciferases, and a quench reagent for firefly luciferase is likely to quench other beetle luciferases. Likewise, generally, a quench reagent for an enzyme that catalyzes a particular reaction, e.g., a peroxidase or a phosphatase, is likely to quench other enzymes that catalyze that reaction, i.e., other peroxidases and other phosphatases, respectively.

In the assays of the invention, a substrate for a luciferase is present. In one embodiment, the substrate is a substrate for an anthozoan luciferase, e.g., a Renilla luciferase, i.e., a coelenterazine, or a protected coelentrazine, such as EnduRen, ViviRen, and those disclosed in WO 03/040100, the disclosure of which is incorporated by reference herein. In one embodiment, the substrate is a substrate for a beetle luciferase, or a prosubstrate for a beetle luciferase, e.g., D-luciferin or a modified luciferin such as one disclosed in U.S. Pat. No. 7,148,030, and U.S. published application Nos. 2004/0171099 and 2007/0015790, the disclosures of which are incorporated by reference herein. The prosubstrate contains a group that is a substrate for another molecule. When the prosubstrate interacts with the appropriate molecule, e.g., a nonluciferase enzyme, the group is removed to yield a substrate for a beetle luciferase. When the appropriate enzyme is absent, the group is not removed.

Thus, in one embodiment, a modified anthozoan luciferase substrate is employed which is structurally related to the native substrate but is modified to contain a substrate for a different enzyme (a “prosubstrate”). A prosubstrate in the absence of its corresponding enzyme and the presence of the luciferase and appropriate reagents, does not result in luminescence, or results in substantially reduced luminescence relative to the unmodified substrate, but in the presence of the corresponding enzyme and the luciferase and appropriate reagents, yields luminescence, and may be employed in the kits and methods of the invention.

The invention includes the use of luciferin and various derivatives thereof. Certain examples of the luciferin derivatives include, but are not limited to, D-luciferin, aminoluciferin, dihydroluciferin, luciferin 6′-methyl ether, luciferin 6′-chloroethylether, 5′-fluoroluciferin, 7′-fluoroluciferin, 5′,7′-difluoroluciferin, various amino fluoro luciferin derivatives (e.g., 5′,7′-aminofluoroluciferin and 7′,5′-aminofluoroluciferin), and derivatives of coelenterazine such as coelenterazine n, coelenterazine h, coelenterazine c, coelenterazine cp, coelenterazine e, coelenterazine f, coelenterazine fcp, coelenterazine i, coelenterazine icp or coelenterazine 2-methyl, that may be modified to contain substrates for other enzymes; for example, see International Application No. PCT/US03/02936. In one embodiment, coelenterazine or a derivative thereof is a substrate for an anthozoan luciferase, a copepod luciferase, e.g., Gaussia luciferase, a decapod luciferase, e.g., Oplophorus luciferase, or other crustacean luciferase.

Typically, for coelenterazine this derivatization involves the modification of functional groups such as phenol (—C₆H₄—OH), carbonyl (>C═O), and aniline (—C₆H₄—NIH2) with an enzyme-removable blocking group. The blocking group may also cause the functional groups to be less reactive toward their surroundings and thus can be referred to as a protecting group. Possible blocking groups include esters, which can be removed by interaction with esterases. Possible blocking groups also include phosphoryls, which can be removed by interaction with phosphatases, including phosphodiesterases and alkaline phosphatase. Possible blocking groups also include glucosyls, which may be removed by interaction with glycosidases, α-D-galactoside, β-D-galactoside, α-D-glucoside, β-D-glucoside, α-D-mannoside, β-D-mannoside, β-D-fructofuranoside, and β-D-glucosiduronate. One skilled in the art would be able to recognize other enzyme-removable blocking groups that could be used in the invention. Examples of the interaction of enzymes and enzyme-removable groups are described in U.S. Pat. No. 5,831,102, as well as Tsien (1981); Redden et al. (1999); and Annaert et al. (1997).

Enzyme-removable groups may be designed such that they can only be removed by the action of a specific enzyme. For example, certain fatty acids may be used as enzyme-removable groups, and only specific esterases will convert these protected coelenterazines into coelenterazines. A blocking group with high steric hindrance, such as a tert-butyl group, may be used. Such a blocking group could be useful in screening for novel esterases that can act upon bulky, hindered esters. Amino acids may also be used as blocking groups. The protected coelenterazines may be further modified by substituting the enol oxygen atom with a nitrogen atom connected to a protecting group. This type of protecting group could then be removed by a protease, and subsequent hydrolysis of the protected coelenterazine to the enol/carbonyl would provide a coelenterazine.

These enzyme-removable groups are preferably derivatives of alcohol functional groups. In the case of a carbonyl functional group in coelenterazines, derivatization may involve the conversion of the carbonyl to an enol group (—C═C—OH). The carbonyl and enol forms of the coelenterazine may be in a dynamic equilibrium in solution such that there is always a proportion of the substrates that are in the enol form (see the Enol Acylation Scheme below). The hydroxyl (—OH) portion of the enol group can readily be derivatized. Derivatization via ester formation using an acylating agent is illustrated schematically below.

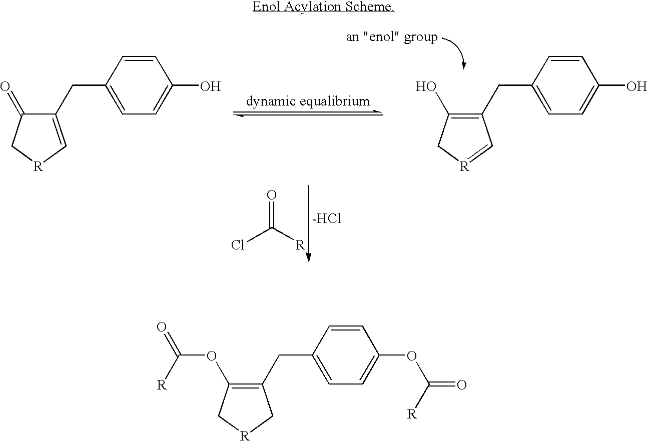

The coelenterazine having structure V-A contains two phenolic groups and one carbonyl group, and any combination of these groups may be protected. Derivatization with ether protecting groups can be carried out, for example, by treating the coelenterazine with an alkylating agent such as acetoxymethyl bromide. Derivatization with ester protecting groups can be carried out for example by treating the coelenterazine with an acylating agent, such as an acetic anhydride or an acetyl chloride. These derivatizations are carried out in basic conditions, that is pH between about 7 and 14. Under these conditions, both the phenolic hydroxyls as well as the imidazolone oxygen can react to form the corresponding esters or ethers. The imidazolone oxygen is believed to react when in the form of the enol. Examples of the protection/deprotection process as well as various protecting groups are described in “Protective Groups in Organic Synthesis”; Eds. Greene and Wuts. John Wiley and Sons, New York, 1991.

One example of the derivatization process is the synthesis of protected coelenterazine V-B from coelenterazine V-A. Protected coelenterazine V-B is also known as triacetyl-coelenterazine due to the presence of three acetyl protecting groups.

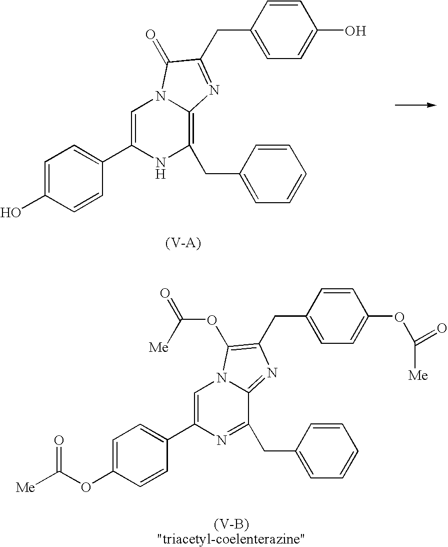

A compound having the structure of compound V-B has reportedly been used as an intermediate in efforts to establish the structure of native coelenterazine V-A (Inoue et al., 1977). It is expected that protected coelenterazine V-B would have fairly low stability relative to other protected coelenterazines, given the lability of the acetyl-derivatized enol group.

For a given protecting group, a derivatized enol is more labile than a similarly derivatized phenol. This increased ability of the enol derivative to react permits the selective hydrolysis of the enol derivative to again provide the imidazolone carbonyl. This type of compound is referred to as a partially protected species since some of the functional groups are protected while others are not. These partially protected species can be used in biological assays, or they can be further reacted with a different acylating or alkylating agent to form an unsymmetrical compound, that is a compound with more than one type of protecting group which also can be used in assays. Selection of the appropriate protecting group may depend on the cell type under consideration and on the desired rate of hydrolysis. The selective hydrolysis can be carried out, for example, as described in Inoue et al. (1977). This is illustrated in the following reaction scheme, for the selective hydrolysis of triacetyl-coelenterazine (VB) to diacetyl-coelenterazine (VC) and subsequent formation of an unsymmetrical protected coelenterazine (VI).

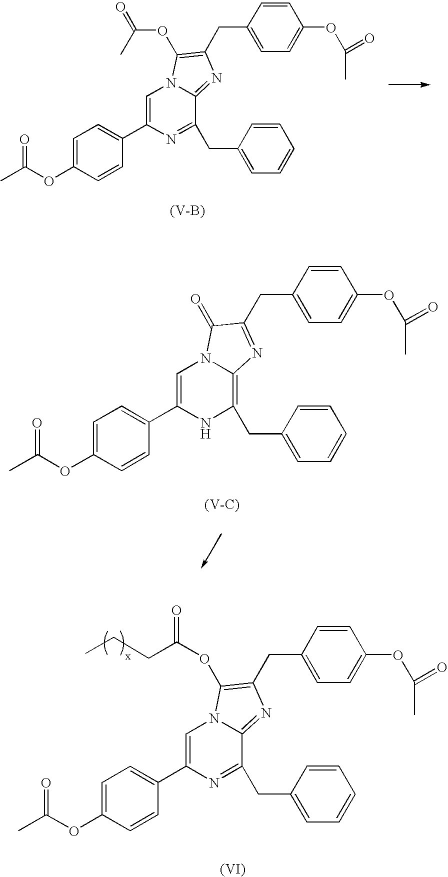

Structures VII-IX illustrate protected coelenterazines having a protecting group on the carbonyl.

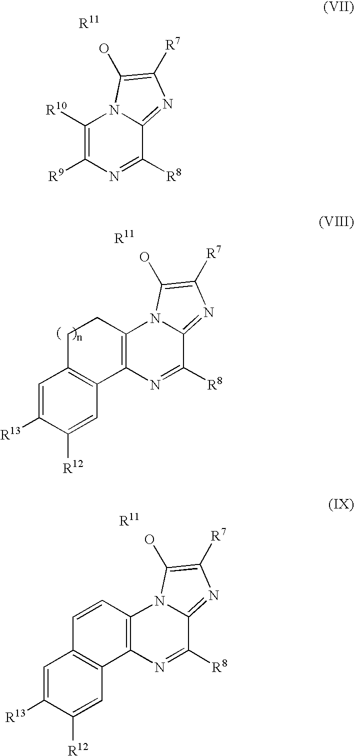

R⁷, R⁸, R⁹ and R¹⁰ can independently be H, alkyl, heteroalkyl, aryl, or combinations thereof. R¹² and R¹³ can independently be —OR¹⁶, H, OH, alkyl, heteroalkyl, aryl, or combinations thereof. For structure VIII, n can be 0, 1, or 2, and preferably 1.

Preferably, R⁷ is as described for R¹ or is —CH₂—C₆H₄OR⁴.

Preferably R⁸ is as described for R², and R¹⁰ is as described for R⁴.

Preferably, R⁹ as described for R³ or is —C₆H₄OR⁵.

R¹¹, R¹⁴, R¹⁵, and R¹⁶, together identified as R^{P}, are protecting groups and can be independently any of a variety of protecting groups. Preferably, these species, together with their corresponding O atom, are ethers, esters, or combinations thereof. For example, the protecting group can be acetyl (R^{P}=—C(═O)—CH₃), butyryl (R^{P}=—C(═O)—C₃H₇), acetoxymethyl (R^{P}=—CH₂—O—C(═O)—CH₃), propanoyloxymethyl (R^{P}—CH₂—O—C(═O)—C₂H₅), butyryloxymethyl (R^{P}=—CH₂—O—C(═O)—C₃H₇), pivaloyloxymethyl (R^{P}=—CH₂—O—C(═O)—C(CH₃)₃), or t-butyryl (R^{P}=—C(═O)—C(CH₃)₃).

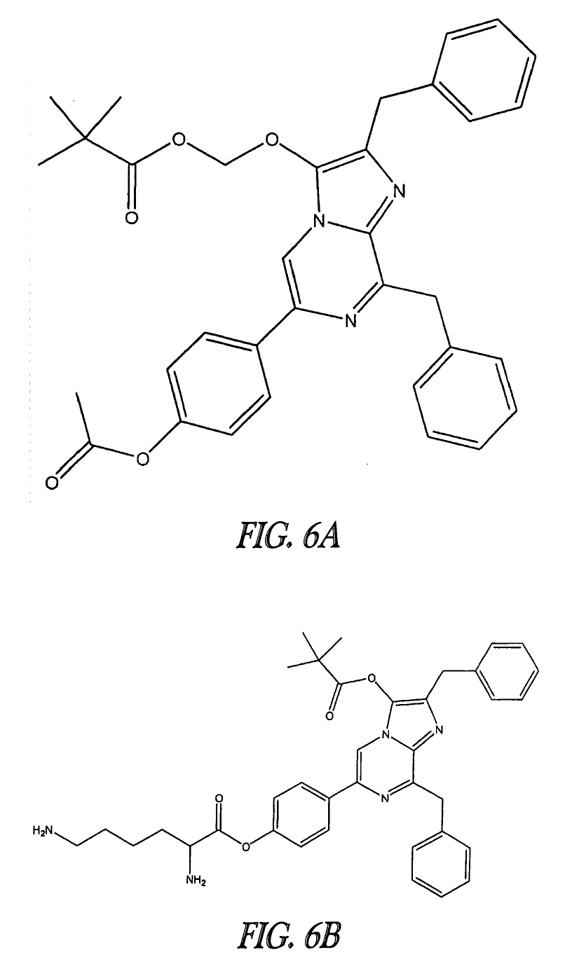

Specific examples of protected coelenterazines include triacetyl-coelenterazine (V-B), tributyryl-coelenterazine (X), diacetyl-coelenterazine-h (XI), acetoxymethyl diacetyl-coelenterazine (XII), acetoxymethyl acetyl-coelenterazine-h (XIII), pivaloyloxymethyl-coelenterazine-h (XIV), acetoxymethyl-dideoxycoelenterazine (XV) (see also FIG. 6)

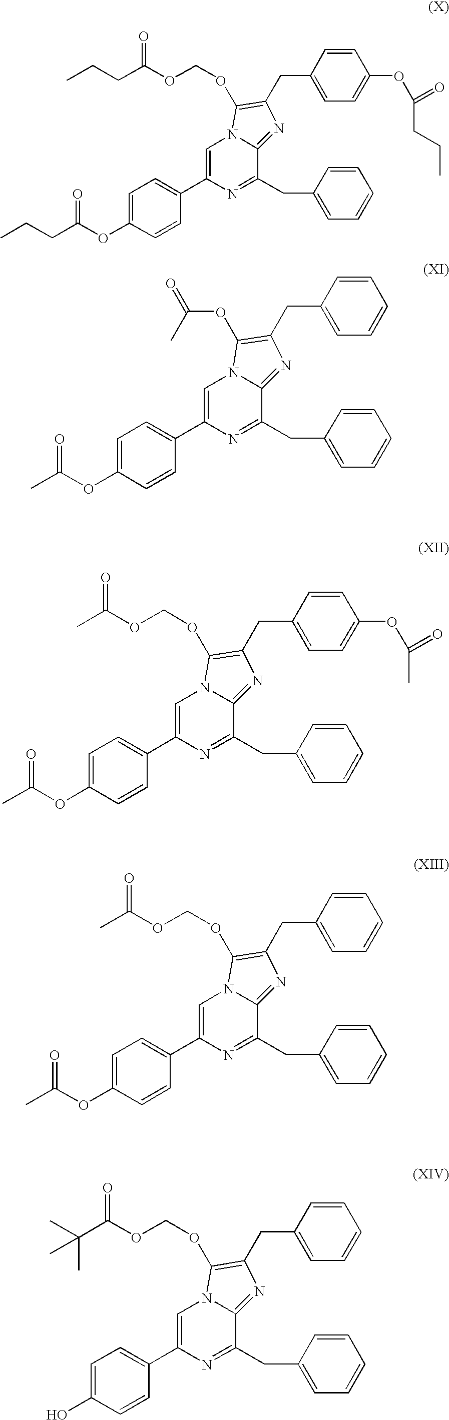

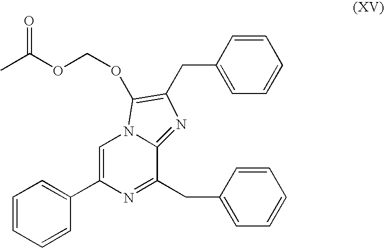

The protecting groups can be removed, and the original functional group restored, when the protected coelenterazine interacts with the appropriate deprotecting enzyme. When the appropriate deprotecting enzyme is absent, the protecting group is not removed and in some embodiments, the protected coelenterazine may be employed as an inhibitor of the luciferase. For ester and ether protecting groups, the deprotecting enzyme can for example be any hydrolase, including esterases. For coelenterazines, having the carbonyl functional group in its deprotected form (i.e., carbonyl) allows for a luminescent interaction with a luminogenic protein, including Renilla luciferase, Oplophorus luciferase, Cypridina luciferase, and aequorin. The protected coelenterazine may only need to be deprotected at the carbonyl site to be converted into a coelenterazine. The presence of protecting groups on the phenolic hydroxyls may still hinder or prevent a luminescent interaction, however.

For enzymes which employ dioxetane substrates, substrates for the reaction may include, and prosubstrates of the reaction may be structurally related to, a dioxetane-containing substrate having the formula (XVI):

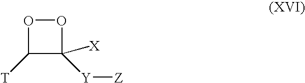

where T is a substituted (i.e., containing one or more (C₁-C₇)alkyl groups or heteroatom groups, e.g. halogens) or unsubstituted cycloalkyl ring (having between 6 and 12 carbon atoms, inclusive, in the ring) or polycycloalkyl group (having 2 or more fused rings, each ring independently having between 5 and 12 carbon atoms, inclusive), bonded to the 4-membered dioxetane ring by a Spiro linkage, e.g., a chloroadamantyl or an adamantyl group, most preferably chloroadamantyl: Y is a fluorescent chromophore, (i.e. Y is group capable of absorbing energy to form an excited, i.e. higher energy, state, from which it emits light to return to its original energy state); X is hydrogen, a straight or branched chain alkyl or heteroalkyl group (having between 1 and 7 carbon atoms, inclusive, e.g., methoxy, trifluoromethoxy, hydroxyethyl, trifluoroethoxy or hydroxypropyl), an aryl group (having at least 1 ring e.g., phenyl), a heteroaryl group (having at least 1 ring e.g., pyrrolyl or pyrazolyl), a heteroalkyl group (having between 2 and 7 carbon atoms, inclusive, in the ring, e.g., dioxane), an aralkyl group (having at least 1 ring e.g., benzyl), an alkaryl group (having at least 1 ring e.g., tolyl), or an enzyme-cleavable group i.e., a group having a moiety which can be cleaved by an enzyme to yield an electron-rich group bonded to the dioxetane, e.g., phosphate, where a phosphorus-oxygen bond can be cleaved by an enzyme, e.g., acid phosphatase or alkaline phosphatase, to yield a negatively charged oxygen bonded to the dioxetane or OR; and Z is hydrogen, hydroxyl, or an enzyme-cleavable group, provided that at least one of X or Z is an enzyme-cleavable group, so that the enzyme cleaves the enzyme-cleavable group, which then leads to the formation of a negatively charged group (e.g., an oxygen anion) bonded to the dioxetane, the negatively charged group causing the dioxetane to decompose to form a luminescencing substance (i.e., a substance that emits energy in the form of light) that includes group Y. The luminescent signal is detected as an indication of the activity of the enzyme. By measuring the intensity of luminescence, the activity of the enzyme can be determined.

An active substrate for a chemiluminescent reaction is generated when X, in formula (XVI), is OR, moiety R is a straight or branched alkyl, aryl, cycloalkyl or arylalkyl of 1-20 carbon atoms. R may include 1 or 2 heteroatoms which may be P, N, S or O. The substituent R is halogenated. The degree of halogenation will vary depending on the selection of substituents on the adamantyl group, on the aryl group, and the desired enzyme kinetics for the particular application envisioned. Most preferably, R is a trihaloalkyl moiety. Preferred groups include trihalo lower alkyls, including trifluoroethyl, trifluoropropyl, heptafluoro butyrol, hexafluoro-2-propyl, a-trifluoromethyl benzyl, α-trifluoromethyl ethyl and difluorochloro butyl moieties. The carbon atoms of substituent R may be partially or fully substituted with halogens. When R is aryl, preferred groups may include a phenyl ring substituted with one or more chloro, fluoro, or trifluoromethyl groups, e.g., 2,5-dichlorophenyl, 2,4-difluorophenyl, 2,3,5-trifluorophenyl, 2-chloro-4-fluorophenyl or 3-trifluoromethyl phenyl. Fluorine and chlorine are particularly preferred substituents, although bromine and iodine may be employed in special circumstances.

Group Y is a fluorescent chromophore or fluorophore bonded to enzyme-cleavable group Z. Y becomes luminescent upon the dioxetane decomposition when the reporter enzyme cleaves group Z, thereby creating an electron-rich moiety which destabilizes the dioxetane, causing the dioxetane to decompose. Decomposition produces two individual carbonyl compounds, one of which contains group T, and the other of which contains groups X and Y. The energy released from dioxetane decomposition causes compounds containing the X and the Y groups to luminesce (if group X is hydrogen, an aldehyde is produced). Y preferably is phenyl or aryl. The aryl moiety bears group Z, as in formula (XVI), and additionally 1-3 electron active groups, such as chlorine or methoxy, as described in U.S. Pat. No. 5,582,980.

Any chromophore can be used as Y. In general, it is desirable to use a chromophore which maximizes the quantum yield in order to increase sensitivity. Therefore, Y usually contains aromatic groups. Examples of suitable chromophores are further detailed in U.S. Pat. No. 4,978,614.

Group Z bonded to chromophore Y is an enzyme cleavable group. Upon contact with an enzyme, the enzyme-cleavable group is cleaved yielding an electron-rich moiety bonded to a chromophore Y; this moiety initiates the decomposition of the dioxetane into two individual carbonyl containing compounds e.g., into a ketone or an ester and an aldehyde if group X is hydrogen. Examples of electron-rich moieties include oxygen, sulfur, and amine or amino anions. The most preferred moiety is an oxygen anion. Examples of suitable Z groups, and the enzymes specific to these groups are given in Table 1 of U.S. Pat. No. 4,978,614. Such enzymes include alkaline and acid phosphatases, esterases, decarboxylases, phospholipase D, β-xylosidase, β-D-fucosidase, thioglucosidase, β-D-galactosidase, α-D-galactosidase, α-D-glucosidase, β-D-glucosidase. β-D-glucouronidase α-D-mannosidase, β-D-mannosidase, β-D-fructofuranosidase, β-D-glucosiduronase, and trypsin.

Dioxetane analogs may also contain one or more solubilizing substituents attached to any of the T, Y and X, i.e., substituents which enhance the solubility of the analogs in aqueous solution. Examples of solubilizing substituents include carboxylic acids, e.g., acetic acid; sulfonic acids, e.g., methanesulfonic acid; and quaternary amino salts, e.g., ammonium bromide; the most preferred solubilizing substituent is methane or ethanesulfonic acid. Other dioxetanes from which dioxetane analogs useful in the practice of this invention may be prepared are described in U.S. Pat. No. 5,089,630; U.S. Pat. No. 5,112,960; U.S. Pat. No. 5,538,847 and U.S. Pat. No. 5,582,980.

Sequestering agents include surfactants and detergents, e.g., those which, in an effective amount, physically separate a substrate, or selectively partition it, from its corresponding enzyme so that, preferably, enzymatic activity is substantially reduced or is eliminated, as well as antibodies or other ligands for the substrate or the enzyme. Preferred sequestering agents include agents which sequester at least a portion, e.g., 35% or more, for instance 50%, 60%, 70%, 80%, 90% or more, of the substrate for a first enzyme, but not a second, distinct enzyme and its corresponding substrate, e.g., into micelles, or shifts the solubility of the first substrate or first enzyme but not that of a second, distinct substrate and its corresponding enzyme, so as to inhibit, e.g., inhibit by at least 35% or more, for instance 50%, 60%, 70%, 80%, 90% or more, an interaction between the first substrate and first enzyme which results in light generation.

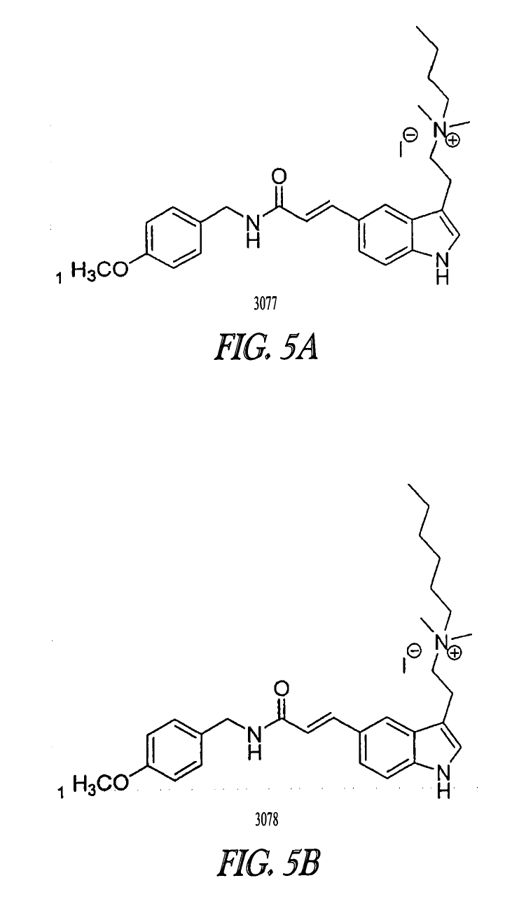

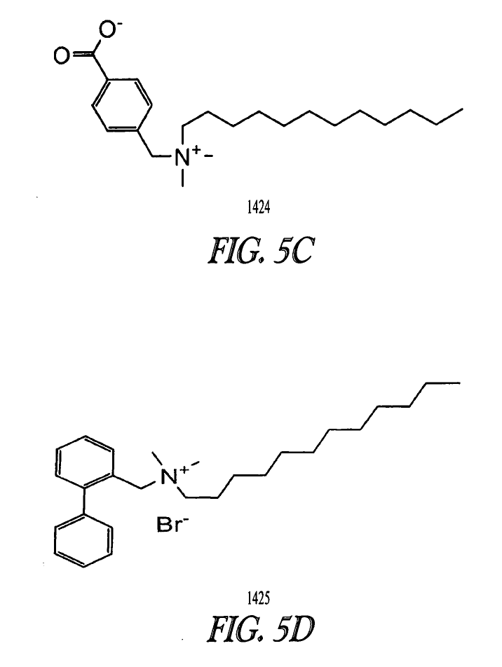

Preferred sequestering agents, include, but are not limited to, anionic, nonionic, amphiteric or cationic detergents or surfactants including those in FIG. 5 of U.S. application publication No. 2004/0224377, which in incorporated herein by reference. In one embodiment, preferred sequestering agents include, but are not limited to, crown ethers, ethoxylated Tomahs such as Tomah E®, azacrown ether, cyclodextran, Tween® 20 (poly(oxyethylene)ₓ-sorbitane-monolaurate), Tween 80, Big Chaps, CHAPS, DTAB, Triton® X-100 (alkylpolyether alcohol; [C₁₆H₂₆O₂]ₙ), and Tergitol®, e.g., Tergitol® NP-9, polyvinylpyrolidone, and glycols, e.g., polyethylene glycol, e.g., 400 or 600. Thus, for instance, the addition of an agent that physically separates a substrate, e.g., a majority of a substrate, from a corresponding enzyme may sequester the substrate (e.g., coelenterazine) in micelles while the enzyme, e.g., Renilla luciferase, remains in the aqueous portion of the solution. In particular, a preferred sequestering agent for a first luminescent reaction is one which physically separates at least a majority of a substrate for a first enzyme which mediates a luminescence reaction from the enzyme, and does not substantially quench the light from a second, distinct enzyme-mediated luminescent reaction.

In one embodiment, the sequestering agent for a first anthozoan luciferase-mediated reaction may be a charged detergent, e.g., about 0.05%, 0.1%, 1.0%, 2% w/v or greater CHAPS, for instance, when a second enzyme-mediated luminescence reaction is mediated by a firefly luciferase such as Ppe2 (WO 01/20002). In another embodiment, the sequestering agent for a first anthozoan luciferase-mediated reaction may be Triton X-100 or Tergitol® NP-9, e.g., 0.01%, 0.05%, 0.1%, 0.5%, 1.0%, 2% and greater Triton X-100 or Tergitol® NP-9, for instance, when a second enzyme-mediated luminescence reaction is mediated by Luc+ (U.S. Pat. No. 5,670,356).

In one embodiment, colored compounds are those which quench blue, green or red light. Compounds may be screened by eye or by absorption spectra to identify candidates for compounds which quench blue, green or red light (see, The Sigma-Aldrich Handbook of Stains, Dyes and Indicators, Green, ed., Aldrich Chemical Company, Milwaukee, Wis., 1990, which is specifically incorporated by reference herein).

Red light as used herein includes light of wavelengths longer than about 590 nm and less than about 730 nm, e.g., wavelengths of 610 nm to 650 nm. Yellow-green light as used herein includes light of wavelengths from about 490 nm to about 590 nm, preferably from about 520 nm to about 570 nm. Yellow light as used herein includes light of wavelengths greater than 560 nm to about 590 nm. Green light as used herein includes light of wavelengths greater than 490 nm to about 560 nm. Blue light as used herein includes light of wavelengths greater than 400 nm to about 490 nm.

For example, red light may correspond to a wavelength of 700 nm, a frequency of 4.29×10¹⁴ Hz or 1.77 eV, as well as to a wavelength of 650 nm, a frequency of 4.62×10¹⁴ Hz or 1.91 eV. Yellow light may correspond to a wavelength of 580 nm, a frequency of 5.16×10¹⁴ Hz or an energy of 2.14 eV. Green light may correspond to a wavelength of 550 nm, a frequency of 5.45×10¹⁴ Hz or an energy of 2.25 eV. Blue light may correspond to a wavelength of 450 nm, a frequency of 6.66×10¹⁴ Hz or an energy of 2.75 eV, while purple light may correspond to a wavelength of 400 nm, a frequency of 7.50×10¹⁴ Hz or an energy of 3.10 eV.

For instance, yellow compounds are useful to quench blue light such as the light emitted by Renilla luciferase- and horseradish peroxidase-mediated reactions. Moreover, yellow compounds do not quench the green-yellow light emitted by some beetle luciferases and so they may be used to quench a dual assay such as a Renilla luciferase/firefly luciferase assay. Certain yellow compounds include, but are not limited to, those which, when dissolved in an aqueous solution, have a peak absorbance within 75 nm of 560 to 590 nm, such as dipyridamole and berberine hemisulfate.

Other compounds that can be used to quench light emitted by Renilla luciferase include, but are not limited to, compounds that absorb blue light and, in one embodiment, may permit yellow-green light to be transmitted, including but not limited to acridine orange, basic orange 21, 4-(4-dimethylaminophenylazo)-benzenene-arsonic acid hydrochloride, 5-aminofluorescein, bis[N,N-bis(carboxymethyl)-aminomethyl]fluoresceine, 2,4-diamino-5-(2-hydroxy-5-nitrophenylazo)-benzenesulfonic acid, Nubian yellow TB, acid orange 10, rosolic acid, napthol yellow S, and solvent yellow 14.

In another embodiment, preferred compounds include compounds which quench red light, e.g., those compounds which, in solution, are cyan or blue colored, including but not limited to azure B tetrafluoroborate, acid blue 93, 5,5′,7-indigo-trisulfinic acid tripotassium salt, cresyl violet acetate, tryptan blue, Twort stain, and lissamine green B. In some embodiments, blue compounds, when dissolved in an aqueous solution, can have a peak absorbance within 75 nm of the range 400 to 490 nm.

Blue compounds for quenching red or yellow, but not blue, light include, but are not limited to, blue chromate dye, isosulfan blue, methylene blue, Coomassie blue, acid blue, orcein, Prussian blue, potassium indigotrisulfonate, alpha-napthophthalein, azure II, oil blue N, patent blue VF, pararoaniline base, rhodanile blue, tetrabromophenol blue, toluidine blue 0, Victoria pure blue BO, Victoria Blue B, alkali blue 6B, alphazurine A, and cyanine dye.

In yet another embodiment, preferred compounds include compounds which quench green light, e.g., those compounds which in solution are magenta or red colored, and, in one embodiment permit red light to be transmitted, including but not limited to, acid blue, acid violet 19, amido naphthol red 6B, and basic red 9. In one preferred embodiment, compounds which quench green light and transmit blue light include acid violet 17, indigo blue, pinacyanol chloride, rhodamine 6G perchlorate, rhodanile blue, pararosanaline base, rose Bengal bis(triethylammonium) salt, and 3,3′-dimethylphenolphthalein. In some embodiments, the compounds, when dissolved in an aqueous solution, can have a peak absorbance within 75 nm of the range 590 to 730 nm.

In one embodiment, suitable compounds useful as a quench reagent for chemiluminescent reactions but not enzyme-mediated luminescence reactions that occur within the enzyme active site include organic compounds (i.e., compounds that comprise one or more carbon atoms), such as those disclosed in U.S. application Ser. No. 09/590,884, the disclosure of which is incorporated by reference herein. Suitable organic compounds can comprise a carbon-sulfur bond or a carbon-selenium bond, for example suitable organic compounds can comprise a carbon-sulfur double bond (C═S), a carbon selenium double bond (C═Se), a carbon-sulfur single bond (C—S), or carbon-selenium single bond (C—Se). Suitable organic compounds can also comprise a carbon bound mercapto group (C—SH) or a sulfur atom bound to two carbon atoms (C—S—C). Preferred compounds are lipophyllic in nature.

Suitable compounds that comprise a carbon sulfur double bond or a carbon selenium double bond include for example compounds of formula (XVII):

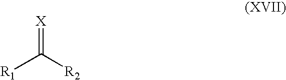

wherein X is S or Se; R₁ and R₂ are each independently hydrogen, (C₁-C₂₀)alkyl, (C₃-C₈)cycloalkyl, (C₁-C₂₀)alkoxy, (C₂-C₂₀)alkenyl, (C₂-C₂₀)alkynyl, aryl, heteroaryl, or NRₐR_{b}; or R₁ and R₂ together with the carbon to which they are attached form a 5, 6, 7, or 8 membered saturated or unsaturated ring comprising carbon and optionally comprising 1, 2, or 3 heteroatoms selected from oxy (—O—), thio (—S—), or nitrogen (—NRC)—, wherein said ring is optionally substituted with 1, 2, or 3 halo, hydroxy, oxo, thioxo, carboxy, (C₁-C₂₀)alkyl, (C₃-C₈)cycloalkyl, (C₁-C₂₀)alkoxy, (C₁-C₂₀)alkanoyl, (C₁-C₂₀)alkoxycarbonyl, (C₂-C₂₀)alkenyl, (C₂-C₂₀)alkynyl, aryl, or heteroaryl; and Rₐ, R_{b} and R_{c} are each independently hydrogen, (C₁-C₂₀)alkyl, (C₃-C₈)cycloalkyl, (C₂-C₂₀)alkenyl, (C₁-C₂₀)alkanoyl, (C₁-C₂₀)alkoxycarbonyl, (C₂-C₂₀)alkynyl, aryl, heteroaryl; wherein any (C₁-C₂₀)alkyl, (C₃-C₈)cycloalkyl, (C₁-C₂₀)alkoxy, (C₂-C₂₀)alkenyl (C₁-C₂₀)alkanoyl, (C₁-C₂₀)alkoxycarbonyl, or (C₂-C₂₀)alkynyl of R₁, R₂, Rₐ, R_{b}, and R_{c} is optionally substituted with one or more (e.g., 1, 2, 3, or 4) halo, hydroxy, mercapto, oxo, thioxo, carboxy, (C₁-C₂₀)alkanoyl, (C₁-C₂₀)alkoxycarbonyl, aryl, or heteroaryl; and wherein any aryl or heteroaryl is optionally substituted with one or more (1, 2, 3, or 4) halo, hydroxy, mercapto, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl; or a salt thereof.

The term “halo” as used herein denotes fluoro, chloro, bromo, or iodo.

The terms “alkyl”, “alkoxy”, “alkenyl”, “alkynyl”, etc. as used herein denote both branched and unbranched groups; but reference to an individual radical such as “propyl” embraces only the straight, unbranched chain radical, a branched chain isomer such as “isopropyl” being specifically referred to.

The term “aryl”, as used herein, denotes a monocyclic or polycyclic hydrocarbon radical comprising 6 to 30 atoms wherein at least one ring is aromatic. Preferably, aryl denotes a phenyl radical or an ortho-fused bicyclic carbocyclic radical having about nine to ten ring atoms in which at least one ring is aromatic. “Heteroaryl” encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of a polycyclic ring comprising 8 to 30 atoms derived therefrom. Preferably, heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

Certain compounds of the invention can be optionally substituted. The term “substituted” indicates that one or more (e.g., 1, 2, 3, 4, or 5; in some embodiments 1, 2, or 3; and in other embodiments 1 or 2) hydrogen atoms on the group indicated in the expression using “substituted” is replaced with a “substituent”, which can be a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, halo, haloalkyl, hydroxy, hydroxyalkyl, aryl, aroyl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, arylsulfinyl, arylsulfonyl, heteroarylsulfinyl, heteroarylsulfonyl, heterocyclesulfinyl, heterocyclesulfonyl, phosphate, sulfate, hydroxylamine, hydroxyl (alkyl)amine, and cyano. Additionally, the suitable indicated groups can include, e.g., —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NRR —S(═O)₂O⁻, —S(═O)₂OH, —S(═O)₂R, —OS(═O)₂OR, —S(═O)₂NR, —S(═O)R, —OP(═O)O₂RR, —P(═O)O₂RR, —P(═O)(O⁻)₂, —P(═O)(OH)₂, —C(═O)R, —C(═O)X, —C(S)R, —C(O)OR, —C(O)O⁻, —C(S)OR, —C(O)SR, —C(S)SR, —C(O)NRR, —C(S)NRR, —C(NR)NRR, where each X is independently a halogen (“halo”): F, Cl, Br, or I; and each R is independently H, alkyl, aryl, heteroaryl, heterocycle, or a protecting group. As would be readily understood by one skilled in the art, when a substituent is keto (═O) or thioxo (═S), or the like, then two hydrogen atoms on the substituted atom are replaced. In some embodiments, one or more of the substituents above are excluded from the group of potential values for substituents on the substituted group.

Suitable compounds that comprise a mercapto group include for example compounds of the formula R₃SH wherein: R₃ is (C₁-C₂₀)alkyl, (C₃-C₈)cycloalkyl, (C₂-C₂₀)alkenyl, (C₂-C₂₀)alkynyl, aryl, or heteroaryl; wherein any (C₁-C₂₀)alkyl, (C₃-C₈)cycloalkyl, (C₂-C₂₀)alkenyl, or (C₂-C₂₀)alkynyl of R₃ is optionally substituted with one or more (e.g., 1, 2, 3, or 4) halo, hydroxy, mercapto oxo, thioxo, carboxy, (C₁-C₂₀)alkanoyl, (C₁-C₂₀)alkoxycarbonyl, aryl, heteroaryl, or NR_{d}Rₑ; wherein R_{d} and Rₑ are each independently hydrogen, (C₁-C₂₀)alkyl, (C₃-C₈)cycloalkyl, (C₂-C₂₀)alkenyl, (C₂-C₂₀)alkynyl, (C₁-C₂₀)alkanoyl, (C₁-C₂₀)alkoxycarbonyl aryl, or heteroaryl; and wherein any aryl or heteroaryl is optionally substituted with one or more (1, 2, 3, or 4) halo, mercapto, hydroxy, oxo, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl; or a salt thereof.

Other suitable compounds include for example compounds of the formula R₄NCS wherein: R₄ is (C₁-C₂₀)alkyl, (C₃-C₈)cycloalkyl, (C₂-C₂₀)alkenyl, (C₂-C₂₀)alkynyl, aryl, or heteroaryl; wherein any (C₁-C₂₀)alkyl, (C₃-C₈)cycloalkyl, (C₂-C₂₀)alkenyl, or (C₂-C₂₀)alkynyl of R₃ is optionally substituted with one or more (e.g 1, 2, 3, or 4) halo, hydroxy, mercapto oxo, thioxo, carboxy, (C₁-C₂₀)alkanoyl, (C₁-C₂₀)alkoxycarbonyl, aryl, heteroaryl, or NR_{f}R_{g}; wherein R_{f} and R_{g} are each independently hydrogen, (C₁-C₂₀)alkyl, (C₃-C₈)cycloalkyl, (C₂-C₂₀)alkenyl, (C₂-C₂₀)alkynyl, (C₁-C₂₀)alkanoyl, (C₁-C₂₀)alkoxycarbonyl aryl, or heteroaryl; and wherein any aryl or heteroaryl is optionally substituted with one or more (1, 2, 3, or 4) halo, mercapto, hydroxy, oxo, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl; or a salt thereof.

Other suitable compounds that comprise a carbon-selenium single bond or a carbon sulfur single bond include compounds of formula R₅—X—R₆ wherein:

X is —S— or —Se—;

R₅ is (C₁-C₂₀)alkyl, (C₃-C₈)cycloalkyl, (C₂-C₂₀)alkenyl, (C₂-C₂₀)alkynyl, aryl, or heteroaryl; and R₆ is hydrogen, (C₁-C₂₀)alkyl, (C₃-C₈)cycloalkyl, (C₂-C₂₀)alkenyl, (C₂-C₂₀)alkynyl, aryl, or heteroaryl;

or R₅ and R₆ together with X form a heteroaryl;

wherein any (C₁-C₂₀)alkyl, (C₃-C₈)cycloalkyl, (C₂-C₂₀)alkenyl, or (C₂-C₂₀)alkynyl of R₅ or R₆ is optionally substituted with one or more (e.g 1, 2, 3, or 4) halo, hydroxy, mercapto oxo, thioxo, carboxy, (C₁-C₂₀)alkanoyl, (C₁-C₂₀)alkoxycarbonyl, aryl, heteroaryl, or NRₖRₘ;

wherein Rₖ and Rₘ are each independently hydrogen, (C₁-C₂₀)alkyl, (C₃-C₈)cycloalkyl, (C₂-C₂₀)alkenyl, (C₂-C₂₀)alkynyl, (C₁-C₂₀)alkanoyl, (C₁-C₂₀)alkoxycarbonyl aryl, or heteroaryl; and

wherein any aryl or heteroaryl is optionally substituted with one or more (1, 2, 3, or 4) halo, mercapto, hydroxy, oxo, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl; or a salt thereof.

Specific and preferred values listed below for radicals, substituents, and ranges, are for illustration only; they do not exclude other defined values or other values within defined ranges for the radicals and substituents.

Specifically, (C₁-C₂₀)alkyl can be methyl, ethyl, propyl, isopropyl, butyl, iso-butyl, sec-butyl, tert-butyl, pentyl, 3-pentyl, or hexyl; (C₃-C₈)cycloalkyl can be cyclopropyl, cyclobutyl, cyclopentyl, or cyclohexyl; (C₁-C₂₀)alkoxy can be methoxy, ethoxy, propoxy, isopropoxy, butoxy, iso-butoxy, sec-butoxy, pentoxy, 3-pentoxy, or hexyloxy; (C₂-C₂₀)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; (C₂-C₂₀)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₂₀)alkanoyl can be acetyl, propanoyl or butanoyl; (C₁-C₂₀)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₂-C₂₀)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide). Each of these groups can be optionally substituted, as described above.

Specifically, R₁ and R₂ can each independently be hydrogen, (C₁-C₂₀)alkyl, (C₃-C₈)cycloalkyl, (C₂-C₂₀)alkenyl, (C₂-C₂₀)alkynyl, aryl, heteroaryl, or NRₐR_{b}; wherein Rₐ and R_{b} are each independently hydrogen, (C₁-C₂₀)alkyl, (C₃-C₈)cycloalkyl, (C₂-C₂₀)alkenyl, (C₁-C₂₀)alkanoyl, (C₁-C₂₀)alkoxycarbonyl, (C₂-C₂₀)alkynyl, aryl, or heteroaryl; wherein any (C₁-C₂₀)alkyl, (C₃-C₈)cycloalkyl, (C₁-C₂₀)alkoxy, (C₂-C₂₀)alkenyl (C₁-C₂₀)alkanoyl, (C₁-C₂₀)alkoxycarbonyl, or (C₂-C₂₀)alkynyl of R₁, R₂, Rₐ, and R_{b} is optionally substituted with 1 or 2 halo, hydroxy, mercapto, oxo, thioxo, carboxy, (C₁-C₂₀)alkanoyl, (C₁-C₂₀)alkoxy-carbonyl, aryl, or heteroaryl; and wherein any aryl or heteroaryl is optionally substituted with one or more halo, hydroxy, mercapto, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl.

Specifically, R₁ and R₂ can each independently be hydrogen, (C₁-C₁₀)alkyl, (C₂-C₁₀)alkenyl, (C₂-C₁₀)alkynyl, aryl, or NRₐR_{b}.

Specifically, R₁ and R₂ together with the carbon to which they are attached can form a 5 or 6 membered saturated or unsaturated ring comprising carbon and optionally comprising 1 or 2 heteroatoms selected from oxy (—O—), thio (—S—), or nitrogen (—NR_{c})—, wherein said ring is optionally substituted with 1, 2, or 3 halo, hydroxy, oxo, thioxo, carboxy, (C₁-C₂₀)alkyl, (C₃-C₈)cycloalkyl, (C₁-C₂₀)alkoxy, (C₁-C₂₀)alkanoyl, (C₁-C₂₀)alkoxycarbonyl, (C₂-C₂₀)alkenyl, (C₂-C₂₀)alkynyl, aryl, or heteroaryl; wherein R_{c} is hydrogen, (C₁-C₂₀)alkyl, (C₃-C₈)cycloalkyl, (C₂-C₂₀)alkenyl, (C₁-C₂₀)alkanoyl, (C₁-C₂₀)alkoxycarbonyl, (C₂-C₂₀)alkynyl, aryl, heteroaryl; wherein any (C₁-C₂₀)alkyl, (C₃-C₂₀)cycloalkyl, (C₁-C₂₀)alkoxy, (C₂-C₂₀)alkenyl (C₁-C₂₀)alkanoyl, (C₁-C₂₀)alkoxycarbonyl, or (C₂-C₂₀)alkynyl of R₁, R₂, and R_{c} is optionally substituted with one or more halo, hydroxy, mercapto, oxo, thioxo, carboxy, (C₁-C₂₀)alkanoyl, (C₁-C₂₀)alkoxycarbonyl, aryl, or heteroaryl; and wherein any aryl or heteroaryl is optionally substituted with one or more halo, hydroxy, mercapto, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl.

Specifically, R₁ and R₂ can each independently be NRₐR_{b}; wherein Rₐ and R_{b} are each independently hydrogen, (C₁-C₂₀)alkyl, (C₃-C₈)cycloalkyl, (C₂-C₂₀)alkenyl, (C₁-C₂₀)alkanoyl, (C₁-C₂₀)alkoxycarbonyl, (C₂-C₂₀)alkynyl, aryl, heteroaryl; wherein any (C₁-C₂₀)alkyl, (C₃-C₈)cycloalkyl, (C₂-C₂₀)alkenyl (C₁-C₂₀)alkanoyl, (C₁-C₂₀)alkoxycarbonyl, or (C₂-C₂₀)alkynyl is optionally substituted with one or more halo, hydroxy, mercapto, oxo, thioxo, carboxy, aryl, or heteroaryl; and wherein any aryl or heteroaryl is optionally substituted with one or more halo, hydroxy, mercapto, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl.

Specifically, R₁ and R₂ can each independently be amino, (C₁-C₂₀)alkyl, (C₁-C₂₀)alkylamino, allylamino, 2-hydroxyethylamino, phenylamino, or 4-thiazoylamino.

Specifically, R₁ and R₂ can each independently be amino, methyl, allylamino, 2-hydroxyethylamino, phenylamino, or 4-thiazoylamino.

A specific value for R₃ is (C₁-C₂₀)alkyl optionally substituted with one or more halo, mercapto oxo, thioxo, carboxy, (C₁-C₂₀)alkanoyl, (C₁-C₂₀)alkoxy-carbonyl, aryl, heteroaryl, or NR_{d}Rₑ.

A specific value for R₃ is 2-aminoethyl, 2-amino-2-carboxyethyl, or 2-acylamino-2-carboxyethyl.

A specific value for R₄ is aryl, optionally substituted with one or more halo, mercapto, hydroxy, oxo, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl.

Specifically, R₅ is (C₁-C₁₀)alkyl, (C₃-C₆)cycloalkyl, (C₂-C₁₀)alkenyl, (C₂-C₁₀)alkynyl, aryl, or heteroaryl; and R₆ is hydrogen, (C₁-C₁₀)alkyl, (C₃-C₆)cycloalkyl, (C₂-C₁₀)alkenyl, (C₂-C₁₀)alkynyl, aryl, or heteroaryl.

Specifically, R₅ and R₆ together with X can form a heteroaryl.

Preferred organic compounds exclude polypeptides and proteins comprising one or more mercapto (C—SH) groups.

Preferred organic compounds exclude compounds that comprise one or more mercapto (C—SH) groups.

In one embodiment, preferably the quench reagent is not iodide, iodine, sulfate, nitrate, iso-propanol, 2-(4-aminophenyl)-6-methylbenzothiazole (APBNH), dimethyldecylphosphine oxide, pyrophosphate, benzothiazole, 2-phenyl-benzothiazole, n-butanol, trans-1,2,-diaminocyclohexane-N,N,N′,N′-tetraacetic acid (CDTA), 2-(6′-hydroxy-2′-benzothiazolyl)-thiazole-4-carboxylic acid, ethylenediaminetetrethylenediaminetetraacetic acid, 2(o-hydroxyphenyl)-benzothiazole, adenosine 5′-triphosphate, 2′,3′-acyclic dialcohol periodate oxidized borohydride reduced, sodium dodecyl sulfate (SDS), citric acid, Tween® 20, or Triton® X-100. In another embodiment, the composition comprising the quench reagent does not comprise citric acid, n-butanol, isopropanol, ethanol, iodide, iodine, Tween® 20, Triton® X-100, cetyl trimethyl ammonium bromide, or any combination thereof. In one embodiment, the quench reagent is not a thiol.

The invention also includes single reporter and dual reporter assay kits which contain one or more selective quench reagents. The single reporter kit comprises at least one selective quench reagent composition, e.g., one capable of quenching photon emission from an anthozoan luciferase-mediated luminescence reaction. The at least one selective quench reagent composition is disposed within a suitable first container. At least one functional enzyme substrate for the anthozoan luciferase-mediated luminescence reaction is optionally included in the kit, along with a suitable second container into which the at least one functional enzyme substrate is disposed. The kit may include instructions on its use.

In one embodiment, two or more selective quench reagents are employed in compositions and kits of the invention. In one embodiment, their combined effect on quenching is more than additive.

The dual reporter kit includes at least one selective quench reagent capable of quenching photon emission from an anthozoan luciferase-mediated luminescence reaction but not capable of substantially quenching at least one second and distinct enzyme-mediated luminescence reaction. Alternatively, or in addition to at least one selective quench reagent, the kit includes a quench-and-activate composition comprising at least one first quench reagent capable of selectively quenching photon emission from an anthozoan luciferase-mediated luminescence reaction but not capable of substantially quenching photon emission from a second and distinct enzyme-mediated luminescence reaction.

The at least one selective quench reagent composition, or the quench-and-activate composition, is disposed within a suitable first container. At least one functional enzyme substrate for the anthozoan luciferase-mediated luminescence reaction is contained within a suitable second container. Optionally, the dual reporter kit comprises at least one functional enzyme substrate for the second enzyme-mediated luminescence reaction contained within a suitable third container. The dual reporter kit may include instructions for its use. Also optionally, the dual reporter kit may also contain at least a second quench reagent, which is different than the first selective quench reagent, contained within a suitable third container. The second quench reagent, which may be a selective quench reagent, is capable of quenching the second and distinct enzyme-mediated luminescent reaction.

The invention also includes assay kits for carrying out the methods of the invention. Such kits comprise, in one or more containers, usually conveniently packaged to facilitate use in assays, quantities of various compositions for carrying out the methods. Thus, in kits for assaying an anthozoan luciferase and a beetle luciferase, a substrate for the luciferase, or ATP, for the anthozoan luciferase, e.g., a Renilla luciferase, the kit may include a composition having a reagent buffer, e.g., at pH 5 or pH 7, high salt, e.g., about 0.5 M KCl or NaCl, a substrate such as coelenterazine or coelenterazine hh, and may comprise other components, e.g., the anthozoan luciferase. For the beetle luciferase reaction, there may be a composition that may contain one or more or any combination of the following: magnesium ion, ATP, beetle luciferase, luciferin, and/or a thiol reagent. The luciferin may be a luciferin derivative, such as fluoroluciferin. The luciferin derivative, e.g., fluoroluciferin, may be more stable at acidic pH. Nevertheless, the reaction may be conducted at a pH of about 6.0 to 8.5, e.g., 7.0 to 7.7, e.g., a pH of about 7.4.

In one embodiment, a composition for a beetle luciferase may comprise both CoA and a thiol reagent, such as dithiothreitol (DTT), other than CoA, and may comprise other components, such as, for example, a proteinaceous luciferase-activity enhancer (e.g., bovine serum albumin or glycol in purified enzyme preparations), EDTA or CDTA, a phosphate salt or 2-aminoethanol, or a buffer to provide a solution at a pH and ionic strength at which the beetle luciferase-luciferin reaction will proceed at a suitable rate.

One component of such kits and compositions may be a cation, e.g., magnesium, calcium, manganese and the like.

The reactions may include a reducing agent, such as a thiol reagent. Thiol reagents that may be used in the methods and compositions of the invention are CoA or thiol reagents other than CoA. The thiol reagents other than CoA are reagents which have a free sulfhydryl group that is capable of being effective as a reducing agent in an air-saturated aqueous solution under conditions, of temperature, pH, ionic strength, chemical composition, and the like, at which the reaction occurs. Among these reagents is DTT. Among others which can be employed are beta-mercaptoethanol, 2-mercaptopropanol (either enantiomer or both enantiomers in any combination), 3-mercaptopropanol, 2,3-dithiopropanol, and glutathione. However, non-thiol reducing agents may be present in the reaction, which reaction may lack or contain a thiol containing reagent.

The assay kits may also include one or more substrates, e.g., a substrate for the first reaction and a substrate for the second reaction, e.g., a substrate for an enzyme that yields a product which is a substrate for a luminescence reaction. The substrate may be prepared synthetically. For instance, modified forms of coelenterazine or other luciferins, “blocked” or “protected” forms, as described herein may be employed in the kits and methods of the invention. Blocked luciferins such as blocked coelenterazine include modified forms of luciferin that no longer interact with a luciferase to yield luminescence.

In one embodiment, the modification is the addition of any enzyme-removable group to the coelenterazine or luciferin and the interaction of the blocked coelenterazine or luciferin with an appropriate enzyme yields an active substrate capable of luminescence. The enzyme which converts the blocked coelenterazine or luciferin into an active luciferin is preferably a non-luminogenic enzyme. All of the coelenterazines disclosed in WO 03/040100, the disclosure of which is incorporated by reference herein, may be converted into blocked coelenterazines. Exemplary modified luciferins having an enzyme-removable group are disclosed in U.S. Pat. No. 7,148,030, and U.S. application Ser. Nos. 10/665,314 and 11/444,145.

The various components described above can be combined, e.g., in solution or a lyophilized mixture, in a single container or in various combinations (including individually) in a plurality of containers. In one kit for assaying for an enzyme, substrate or cofactor via an enzyme-mediated luminescence reaction in cells in which the enzyme, cofactor or substrate may be present, a solution (or the components for preparing a solution) useful for lysing the cells while preserving (against the action of various enzymes released during lysis) the enzyme, substrate or cofactor that might be in the cells in an active form, or a form which can be made active, is included.

The skilled are also aware that compositions including those described herein, and other than those described herein, may be present in any assay reaction mixture, and thus in the kits of the invention, in order to, for example, maintain or enhance the activity of an enzyme or as a consequence of the procedures used to obtain the aliquot of sample being subjected to the assay procedures. Thus, typically buffering agents, such as tricine, HEPPS, HEPES, MOPS, Tris, glycylglycine, a phosphate salt, or the like, will be present to maintain pH and ionic strength; a proteinaceous material, such as a mammalian serum albumin (preferably bovine serum albumin) or lactalbumin or an ovalbumin, that enhances the activity of an enzyme, may be present; EDTA or CDTA (cyclohexylenediaminetetraacetate) or the like, may be present, to minimize enzyme inactivation by toxic metal ions and to suppress the activity of metal-containing proteases or phosphatases that might be present in systems (e.g., cells) from which the reporter to be assayed is extracted and that could adversely affect the reporter or other components of the reaction. Glycerol or ethylene glycol, which stabilize enzymes, might be present.

For instance, counterions to a cation, e.g., magnesium, may be present. As the skilled will understand, the chemical identities and concentrations of these counterions can vary widely, depending on the magnesium salt used to provide the magnesium ion, the buffer employed, the pH of the solution, the substance (acid or base) used to adjust the pH, and the anions present in the solution from sources other than the magnesium salt, buffer, and acid or base used to adjust pH.

In one embodiment, the magnesium ion can be supplied as the carbonate salt, to provide the desired magnesium ion concentration, in a solution with the buffer to be used (e.g., tricine) and then the pH of the buffered solution can be adjusted by addition of a strong acid, such as sulfuric, which will result in loss of most of the carbonate (and bicarbonate) as carbon dioxide and replacement of these anions with sulfate, bisulfate, tricine anion, and possibly also other types of anions (depending on other substances (e.g., phosphate salts) that provide anions and might be present in the solution). Oxygen-saturation from the air of the solution in which the assay method is carried out is sufficient to provide the molecular oxygen required in the luciferase reaction. In any case, it is well within the skill of the ordinarily skilled to readily ascertain the concentrations of the various components in an assay reaction mixture, including the components specifically recited above in the description of the method, that are effective for activity of the luciferase.

The test kits of the invention can also include, as well known to the skilled, various controls and standards, such as solutions of known enzyme, substrate or cofactor, e.g., ATP, concentration, including no enzyme, no substrate or no cofactor (e.g., no ATP which is for a firefly luciferase negative control) solutions, to ensure the reliability and accuracy of the assays carried out using the kits, and to permit quantitative analyses of samples for the analytes (e.g., enzyme, substrate, cofactor and the like) of the kits.

The types of samples which can be assayed in accordance with the method of the invention include, among others, samples which include a luminescent reporter as a genetic reporter, a luminescent reporter as a reporter for a cellular molecule or a modulator of that molecule, a reporter in an immunoassay or a reporter in a nucleic acid probe hybridization assay. As understood in the immunoassay and nucleic acid probe arts, the enzyme assayed in accordance with the present invention is physically, e.g., chemically or recombinantly, linked, by any of numerous methods known in those arts, to an antibody or fragment thereof or nucleic acid probe used in detecting an analyte in an immunoassay or nucleic acid probe hybridization assay, respectively. Then, also following well known methods, the reporter-labeled antibody or nucleic acid probe is combined with a sample to be analyzed, to become bound to a molecule (e.g., antigen or an anti-antigen antibody, in the case of an immunoassay, or a target nucleic acid, in the case of a nucleic acid probe hybridization assay) that is sought to be detected and might be present in the sample and then reporter-labeled antibody or nucleic acid probe that did not become bound to analyte is separated from that, if any, which did become bound.

The reporter can remain physically linked to the labeled antibody or probe during the assay for the reporter in accordance with the present invention or, again by known methods, can be separated from the antibody or nucleic acid probe prior to the assay for the reporter in accordance with the present invention. Immunoassays and nucleic acid probe hybridization assays, in which an enzyme that mediates a luminescence reaction can be used as a reporter or label, have many practical and research uses in biology, biotechnology, and medicine, including detection of pathogens, detection of genetic defects, diagnosis of diseases, and the like.

Another type of sample which can be assayed for the presence of a reporter in accordance with the method of the invention is an extract of cells in which expression of the reporter occurs in response to activation of transcription from a promoter, or other transcription-regulating element, linked to a DNA segment which encodes the reporter, or as a result of translation of RNA encoding the reporter. In such cells, luminescent reporters are used, similarly to the way other enzymes, such as chloramphenicol acetyltransferase, have been used to monitor genetic events such as transcription or regulation of transcription. Such uses of luminescent reporters are of value in molecular biology and biomedicine and can be employed, for example, in screening of compounds for therapeutic activity by virtue of transcription-activating or transcription-repressing activity at particular promoters or other transcription-regulating elements.

For instance, in a dual assay, a sample containing two distinct enzymes, such as firefly luciferase and a Renilla luciferase, or any combination of distinct molecules which are capable of being detected by distinct enzyme-mediated luminescence reaction, e.g., a protease and ATP, is assayed. In one embodiment, the assay is a coupled assay, e.g., a prosubstrate for a luciferase having a protease substrate is contacted with a sample suspected of having the protease, where the sample includes cells expressing the luciferase or includes exogenously added luciferase.

A sample includes a non-cellular sample, e.g., a sample with purified enzymes, an in vitro translation reaction or an in vitro transcription/translation reaction, a cellular (intact) sample, either a prokaryotic or eukaryotic sample, or a cellular lysate. In one embodiment, an activating (initiating) agent for one of the two enzyme-mediated reactions is added to the sample, in a vessel such as a well in a multi-well plate and the resulting luminescence measured. A specific quench-and-activate reagent is then added to the well so as to selectively quench the first enzyme-mediated reaction, and simultaneously activate the second enzyme-mediated reaction.

Alternatively, the selective quench reagent and a second light activating reagent specific for the second enzyme-mediated luminescence reaction can be added to the sample sequentially. The luminescence from the second reaction is then measured in the same manner as the first. Optionally, luminescence from the sample may then be quenched by adding a second quench reagent, e.g., a nonselective quench reagent or a selective quench reagent for the second enzyme-mediated reaction to the sample. In this manner, the present invention affords a multiplex luminescence assay capable of measuring two distinct parameters within a single sample.

As noted above, one of the enzyme-mediated reactions can act as an internal standard, while the other of the enzyme-mediated reactions may function as a genetic marker or other experimental variable, or alternatively, each reaction can measure a different experimental variable. Moreover, as the skilled will understand, the method of the invention, being an assay method, will usually be carried out with suitable controls or standards (e.g., a sample being analyzed will be analyzed in parallel with solutions with no enzyme and with known concentrations of enzyme) and, with appropriate standards, the method can be adapted to quantitating the concentration of the molecules to be detected in a test sample (i.e., a sample being analyzed).

In compositions of the invention, e.g., those used in methods of the invention, which are aqueous solutions, the substrate is typically present in a concentration of about 0.01 μM to about 2 mM. For firefly luciferase, luciferin saturates at about 0.47 mM in a reagent optimized for maximal light output and at about 1 mM in a reagent optimized for stable signal. For Renilla luciferase, coelenterazine saturates at about 2 μM in a reagent optimized for maximal light output and at about 60 to 100 μM in a reagent optimized for stable signal. In compositions in which ATP is present, the ATP concentration ranges from about 0.01 mM to about 15 mM, preferably about 0.5 mM. When CoA is present in such compositions which are aqueous solutions, the concentration of CoA ranges from about 0.001 mM to about 10 mM, preferably about 0.2 mM to 4 mM. Similarly, the concentration of DTT present is from about 5 mM to about 200 mM, or about 20 to 40 mM.

For sequential Renilla luciferase and beetle luciferase assays, the 100% control value for Reporter #1, the Renilla luciferase-mediated luminescent reaction, may be determined by quantifying light emission from the reaction prior to addition of the quench reagent(s). The 100% control value for Reporter #2, e.g., a firefly luciferase-mediated luminescent reaction, may be determined by quantifying light emission from a reaction which does not contain the quench reagent(s) and does not contain a substrate for Reporter #1.

Thus, the invention provides a series of general and selective inhibitors, for example, inhibitors of Renilla luciferase. The activity of the inhibitors may be non-competitive and may be reversible (e.g., by dilution). Accordingly, the invention provides compounds and uses of compounds of formula (I):

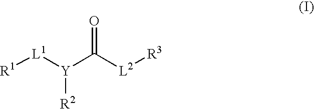

wherein

Y is N or O;

L¹ is (C₁-C₆)alkylene or a direct bond;

R¹ is alkyl, aryl, heteroaryl, or heterocycle;

R² is H, (C₁-C₆)alkyl, or absent;

or L¹ is a direct bond and R¹ and R² together with the nitrogen attached to R² form a heteroaryl or heterocycle group;

L² is optionally unsaturated straight chain or branched (C₁-C₆)alkylene or (C₁-C₆)alkylene-O—;

R³ is alkyl, aryl, heteroaryl, heterocycle;

wherein any alkyl, alkylene, aryl, heteroaryl, or heterocycle is optionally substituted with one to five alkyl, alkenyl, alkoxy, alkanoyl, alkanoyloxy, alkoxycarbonyl, cycloalkyl, cyano, nitro, halo, hydroxy, mercapto, oxo, —SOₙR⁴, N(R^{X})(R^{Y}), N(R^{X})(R^{Y})alkyl, N⁺(R^{X})(R^{Y})(R^{Z}), or N⁺(R^{X})(R^{Y})(R^{Z})alkyl groups wherein R^{X}, R^{Y}, and R^{Z} are each independently H, alkyl, aryl, heteroaryl, or heterocycle; each substituent is optionally substituted with one to three R³ groups; and any nitrogen atom of a nitrogen heterocycle is optionally substituted with an optionally substituted alkyl or acyl group, or with a nitrogen protecting group;

n is 0, 1, 2, or 3; and

R⁴ is H, alkyl, or aryl;

or a salt thereof. In various embodiments, the R³ is heteroaryl substituted with at least one quaternary ammonium group. In some embodiments, R³ is heteroaryl comprising at least one quaternary amine-containing substituent. Thus, the compound of formula (I) can be a compound that is, in at least one position of the ring or at one position on a substituent, substituted with a quaternary ammonium group. In various embodiments, the compound of formula (I) is not 3-(3-(2-(dimethylamino)ethyl)-1H-indol-5-yl)-N-(4-methoxybenzyl)acrylamide. In some embodiments, the salt of formula I is a (C₁-C₂₀)alkyl halide salt. The invention further provides methods for their preparation and methods for their use.

In one embodiment, Y is N. In another embodiment, Y is O.

In one embodiment, L¹ can be (C₁-C₂)alkylene. In another embodiment, L¹ can be —CH₂—.

In various embodiments, R¹ is aryl optionally substituted with one or two alkoxy, nitro, or N(R^{x})(R^{Y}) groups.

In various embodiments, R² is H.

In various embodiments, L¹ is a direct bond and R¹ and R² together with the nitrogen attached to R² form a heterocycle group. The heterocycle can be a morpholino or piperizino group.

In one embodiment, L² is —CH═CH—. In other embodiments, L² can be a 1,1-disubstituted ethene group, or a substituted 1,2-ethene group.

In various embodiments, R³ can be a heterocycle group. R³ can be an optionally substituted indolyl group. The indolyl group can be attached to the compound of formula (I) at the indolyl 5-position. The indolyl group can be substituted at its 3-position with various substituents. The indolyl group can be substituted with, for example, a N(R^{x})(R^{Y})alkyl- or N⁺(R^{X})(R^{Y})(R^{Z})alkyl-group. The N(R^{x})(R^{Y})alkyl-group can be dimethylaminomethyl-, dimethylaminoethyl-, or dimethylaminopropyl-. The N⁺(R^{X})(R^{Y})(R^{Z})alkyl-group can be dimethyl((C₁-C₁₀)alkyl)ammonium ethyl-. In other embodiments, the N⁺(R^{X})(R^{Y})(R^{Z})alkyl-group can form an ion pair with a halide. In other embodiments, the counterion can be various organic or inorganic counterions, such as citrate, maleate, and fumarate, and chlorate, nitrate, and phosphate.

The compound of formula (I) can be a compound of formula (II):

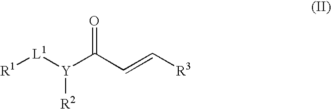

wherein L¹, R¹, R², and R³ are as defined above for formula (I); or a salt thereof. In one embodiment, R³ is a heterocycle comprising a quaternary ammonium substituent, such as a trialkylammonium group at an indole 3-position, or a trialkylammomium alkyl group at any position of the heterocycle, such as an indole 2-, 3-, 4-, 5-, 6-, or 7-position.

The compound of formula (I) also can be a compound of formula (III):

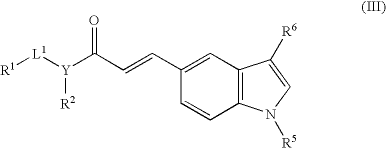

wherein L¹, R¹, and R² are as defined above for formula (I);

R⁵ is H, alkyl, for example, (C₁-C₁₀)alkyl, aralkyl, such as benzyl or substituted aralkyl, aryl, such as phenyl, naphthyl, and substituted versions thereof, or a nitrogen protecting group; and

R⁶ is an N(R^{X})(R^{Y})alkyl- or N⁺(R^{X})(R^{Y})(R^{Z})alkyl-group; or a salt thereof. In one embodiment, R⁶ is a N⁺(R^{X})(R^{Y})(R^{Z})alkyl-group.

In certain specific embodiments, the compounds of formula (I) can include (E)-N-(4-(dimethylamino)benzyl)-3-(3-(2-(dimethylamino)ethyl)-1H-indol-5-yl)acrylamide (3043); (E)-3-(3-(2-(dimethylamino)ethyl)-1-methyl-1H-indol-5-yl)-N-(4-methoxybenzyl)acrylamide (3049); (E)-N-(2,4-dimethoxybenzyl)-3-(3-(2-(dimethylamino)ethyl)-1H-indol-5-yl)acrylamide (3051); (E)-3-(3-(2-(dimethylamino)ethyl)-1H-indol-5-yl)-N-(4-methoxyphenethyl)acrylamide (3062); (E)-3-(3-(2-(dimethylamino)ethyl)-1H-indol-5-yl)-N-(4-nitrobenzyl)acrylamide (3063); (E)-3-(3-(2-(dimethylamino)ethyl)-1H-indol-5-yl)-1-morpholinoprop-2-en-1-one (3064); (E)-3-(3-((dimethylamino)methyl)-1H-indol-5-yl)-N-(4-methoxybenzyl)acrylamide (3067); (E)-3-(3-(2-(dimethylamino)ethyl)-1H-indol-5-yl)-N-(4-methoxybenzyl)acrylamide (3068; GR 46611); (E)-N-(2-(5-(3-(4-methoxybenzylamino)-3-oxoprop-1-enyl)-1H-indol-3-yl)ethyl)-N,N-dimethyloctan-1-ammonium iodide (3070); (E)-3-(3-(3-(dimethylamino)propyl)-1H-indol-5-yl)-N-(4-methoxybenzyl)acrylamide (3071); which are excellent inhibitors of Renilla luciferase.

In other specific embodiments, the compounds of formula (I) can include (E)-2-cyano-3-(3-(2-(dimethylamino)ethyl)-1H-indol-5-yl)-N-(4-methoxybenzyl)acrylamide (3044); (E)-N-(4-methoxybenzyl)-3-(quinolin-6-yl)acrylamide (3046); (E)-3-(1-benzyl-3-(2-(dimethylamino)ethyl)-1H-indol-5-yl)-N-(4-methoxybenzyl)acrylamide (3055); (E)-3-(1H-indol-5-yl)-N-(4-methoxybenzyl)acrylamide (3056); (E)-6-(3-(4-methoxybenzylamino)-3-oxoprop-1-enyl)-1-methylquinolinium iodide (3061); (E)-3-(3-(2-(dimethylamino)ethyl)-1H-indol-5-yl)-N-(3,4,5-trimethoxybenzyl)acrylamide (3072); and (E)-3-(3-(2-(dimethylamino)ethyl)-1H-indol-5-yl)-N-(2,4,6-trimethoxybenzyl)acrylamide (3073), which are inhibitors of Renilla luciferase.

In other specific embodiments, the compounds of formula (I) can include 2-(3-(2-(dimethylamino)ethyl)-1H-indol-5-yloxy)-N-(4-methoxybenzyl)acetamide (3031); (E)-4-methoxybenzyl 3-(3-(2-(dimethylamino)ethyl)-1H-indol-5-yl)acrylate (3045); (E)-3-(3-(2-(dimethylamino)ethyl)-1H-indol-5-yl)-N-hexylacrylamide (3050); N-(4-methoxybenzyl)-2-(quinolin-6-yl)acrylamide (3053); 2-(1H-indol-5-yl)-N-(4-methoxybenzyl)acrylamide (3054); (E)-N-(4-methoxybenzyl)-3-(4-methyl-2-oxo-2H-chromen-6-yl)acrylamide (3057); N-(4-methoxybenzyl)-2-(4-methyl-2-oxo-2H-chromen-6-yl)acrylamide (3058); and (E)-10-bromo-2-cyano-N-(4-methoxybenzyl)dec-2-enamide (3059), which are suitable inhibitors of Renilla luciferase.

Other embodiments of the invention include (C₁-C₂₀)alkyl halide salts of a tertiary amine of any of the preceding specific compounds of formula I, e.g., those described in the three paragraphs immediately above, and substituted versions thereof. Such quaternary ammonium halides can be prepared by combining the compound of formula I with an alkyl halide under suitable reaction conditions, which are commonly known in the art. Suitable reaction conditions include refluxing the compound of formula I in an alcoholic solvent, such as methanol or ethanol, with an alkyl halide. Examples of alkyl halides include, but are not limited to, (C₁-C₂₀)alkyl halide, for example, methyl iodide, ethyl iodide, butyl iodide, hexyl iodide, octyl iodide, decyl iodide, dodecyl iodide, the corresponding bromides and chlorides, and branched alkyl versions thereof.

The invention also provides compounds of formula (IV):

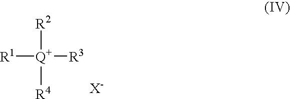

wherein

Q is N or P;

R¹ and R² are each independently alkyl, cycloalkyl, aryl, heteroaryl, heterocycle, arylalkyl;

R³ and R⁴ are each independently (C₁-C₆)alkyl; and

X is an organic or inorganic counterion. The invention further provides methods for their preparation and methods for their use.

In one embodiment, the compound of formula (IV) is an ammonium halide, such as, for example, an ammonium chloride, an ammonium bromide, or an ammonium iodide.

In various embodiments, R¹ can be an arylalkyl group wherein the aryl moiety is a biphenyl group. In one specific embodiment, R¹ can be an N-(biphenyl-2-ylalkyl) group, such as a N-(biphenyl-2-ylmethyl) group.

In various embodiments, R¹ can be alkyl, such as a (C₄-C₂₄)alkyl, a (C₆-C₂₀)alkyl, or a (C₈-C₁₂)alkyl, for example, ethyl, propyl, butyl, pentyl, hexyl, octyl, decyl, or dodecyl. The alkyl groups can be straight chain, branched, or cyclic. In other embodiments, R¹ can be cycloalkyl, such as (C₃-C₈)cycloalkyl, for example, cyclohexyl.

In various embodiments, R² can be an arylalkyl, such as an optionally substituted phenyl(C₁-C₄)alkyl-, or an optionally substituted phenyl(C₁-C₂)alkyl-, for example, benzyl or phenethyl. In certain embodiments, the aryl or heteroaryl component of R² can be substituted with various groups as described above for substituent groups. In one embodiment, the aryl moiety of R² can be substituted with one or more (e.g., 1, 2, 3, 4, or 5) carboxy groups.

In various embodiments, R³ and R⁴ can be alkyl, such as (C₁-C₆)alkyl, more specifically, (C₁-C₄)alkyl, for example, methyl, ethyl, propyl, or butyl. In certain embodiments, R³ and R⁴ can be the same.

In various embodiments, X can be an inorganic counterion, such as halo, for example, fluoride, chloride, bromide, or iodide. In other embodiments, the inorganic counterion can be sulfate or chlorate, and other oxidation states of sulfur and chlorine anions. In yet other embodiments, X can be an organic counterion, such as acetate, carbonate, or an alkyl carboxylate.

In one specific embodiment, the compound of formula (IV) is benzyldodecyldimethylammonium bromide. In other embodiments, the compound of formula (IV) can be N-(biphenyl-2-ylmethyl)-N,N-dimethyldodecan-1-ammonium halide; N-dodecyl-N,N-dimethyl-9H-fluoren-9-ammonium halide; N-benzyl-N,N-dimethylhexan-1-ammonium halide; N-benzyl-N,N-dimethylcyclohexan-ammonium halide; N-benzyl-N,N-diethylethan ammonium halide; or N-benzyl-N,N-diethylethan ammonium halide. In another embodiment, the compound of formula (IV) is N-(biphenyl-2-ylmethyl)-N,N-dimethyldodecan-1-aminium halide, wherein halide is fluoro, chloro, bromo, or iodo. In yet another embodiment, the compound of formula (IV) is 4-((dodecyldimethyl-ammonio)methyl)benzoate.

The invention will be further described by reference to the following non-limiting Examples. The Examples are intended to illustrate various aspects of the invention and should not be construed as to narrow its scope. One skilled in the art will readily recognize that the Examples suggest many other ways in which the present invention could be practiced. It should be understood that many variations and modifications may be made while remaining within the scope of the invention.

### EXAMPLES

The effect of a variety of compounds on firefly (Photinus pyralis) and Renilla (Renilla reniformis) luciferase luminescence was determined to identify compounds which quench Renilla luciferase luminescence without substantially affecting firefly luciferase luminescence. Although inhibitors of Renilla luciferase activity have been reported, they are ineffective in reagents which contain detergents necessary for cell lysis. The Renilla luciferase inhibitors (quenchers) disclosed herein are useful in a Renilla-firefly dual luciferase assay in which Renilla luciferase luminescence is measured followed by the measurement of firefly luciferase luminescence. Thus, the Renilla luciferase quenchers reduce the Renilla luciferase-dependent luminescence after the Renilla luciferase luminescence in a sample has been measured. The quenching of Renilla luciferase prevents the Renilla signal from interfering with the measurement of the firefly luciferase activity in a sample.

### Example 1

Matthews et al. (Biochemistry, 16:5217 (1977)) found that competitive inhibitors for Renilla luciferase invariably were molecules which contained one or more phenyl groups. The inhibitors containing one phenyl group (such as tyramine, phenol and toluene) displayed Kᵢ values ranging from 10⁻⁴ to 10⁻⁵ M, those having two phenyl groups, such as etioluciferin and debenzyl amine, displayed Kᵢ; values between 10⁻⁶ to 10⁻⁷ M, those having two or more phenyl groups had Kᵢ values between 10⁻⁶ to 10⁻⁹ M, and those having three phenyl groups, such as luciferyl sulfate (coelenterazine sulfate) and benzyl oxyluciferin (coelenteramide-h), were observed to have Kᵢ values between 10⁻⁷ and 10⁻⁹ M. The assays described in Matthews et al. were conducted in the absence of cell debris and detergents, thus, the inhibitory effect of agents described in Matthews et al. may be different in the presence of such detergents, which are often present in assays utilizing dual reporters.

Renilla luciferase (1×10⁻⁹ M in Renilla Luciferase Lysis Buffer (Promega))+0.1% Prionex (PentaPharma) was added to Renilla Luciferase Assay Buffer (Promega) containing 3 μM coelenterazine-h. The initial Renilla luciferase-mediated luminescence was measured for 10 seconds using a Turner 20/20 n (Turner BioSystems). A quenching reagent containing 50 mM HEPES pH 7.4, 16 mM MgSO₄, 1 mM CDTA, 0.5% Tergitol NP-9, 0.015% dodecyltrimethylammonium bromide, 10 mM thiourea, 0.05% Mazu-DF-204 (PGP), and 1.5 mM ATP (Pharmacia) was added to the reactions to reduce the Renilla luciferase-mediated luciferase luminescence. To the quenching reagent, the competitive inhibitor methylether coelenterazine-h, methylether coelenterazine-hh or coelenteramide was titrated to either 10 μM or 100 μM in the quenching reagent. After the addition of the inhibitors, a second Renilla luciferase-mediated luminescence reading was taken. Unless otherwise specified, materials were obtained from Sigma-Aldrich.

As the data in Table 1 shows, the inhibitors identified by Matthews et al. are not as effective in the presence of detergents used for cell lysis. The Kᵢ values for each inhibitor in the presence of a lysing detergent and a quenching reagent is much greater (>100 μM versus 0.001 μM; about a 100,000 fold difference). The quenching agents described herein are effective in the presence of detergents used for cell lysis as well as other quenching agents.

TABLE 1

| | Reduction in Renilla | |
|---|---|---|
| | luciferase luminescence | |
| Sample | after 2^{nd} measurement | Kᵢ value |
| Quench Reagent Alone | 18-fold | |
| Quench Reagent + 100 μM | 30-fold | >100 μM |
| methylether coelenterazine-h | | |
| Quench Reagent + 10 μM | 15-fold | |
| methylether coelenterazine-h | | |
| Quench Reagent + 100 μM | 34-fold | about 100 μM |
| methylether coelenterazine-hh | | |
| Quench Reagent + 10 μM | 21-fold | |
| methylether coelenterazine-hh | | |
| Quench Reagent + 100 μM | 21-fold | >100 μM |
| coelenteramide | | |
| Quench Reagent + 10 μM | 18-fold | |
| coelenteramide | | |

### Example 2

To screen for compounds which are able to quench Renilla luciferase luminescence without substantially affecting firefly luciferase luminescence, firefly luciferase and Renilla luciferase luminescence was measured in reactions in which the reagent solutions either did or did not contain a test detergent. The retained luminescence for each luciferase was calculated by dividing the luminescence in the presence of the detergent by the luminescence in the absence of the test detergent. The selectivity value for each of the test detergents was determined by dividing the retained firefly luciferase luminescence by the retained Renilla luciferase luminescence. Those detergents having a selectivity value of 3 or greater are considered highly desirable compounds and selective quenchers of Renilla luciferase-mediated luminescence.

A set of detergents (Table 2; Sigma-Aldrich) was added at 0.5% (from a 5% or 10% stock solution) to either Luciferase Assay Reagent (Promega) or Renilla Luciferase Assay Reagent (Promega) and allowed to equilibrate at room temperature.

The firefly luciferase and Renilla luciferase enzymes were diluted as follows. Firefly luciferase was diluted to 14 ng/ml in Phosphate Buffered Saline (PBS) containing 0.1% of the protein carrier Prionex® (Pentapharm). Renilla luciferase was diluted to 10 ng/ml in 1× Renilla Luciferase Lysis Buffer (Promega) containing 0.1% Prionex®. The Renilla Luciferase Lysis Buffer inhibits the formation of detergent micelles when using the Renilla Luciferase Assay Reagent. In the absence of other detergents, this prevents sequestering of coelenterazine which inhibits Renilla luciferase-mediated luminescence.

The luciferase reactions were initiated by adding 40 μl of either the firefly or Renilla luciferase enzyme solution to 200 μl of the appropriate reagent solution (firefly luciferase enzyme solution+firefly luciferase reagent solution or Renilla luciferase enzyme solution+Renilla luciferase reagent solution, respectively). The reactions were performed in an opaque white, 96-well plate and luminescence was measured for 0.5 seconds per well using a Turner BioSystems Veritas™ Microplate luminometer. The retained firefly luciferase and Renilla luciferase luminescence, as well as the selectivity values for each detergent, were calculated (Table 2). Benzyldimethyldodecylammonium bromide (BDDABr) and dodecyltrimethylammonium bromide were highly effective at selectively quenching Renilla luciferase luminescence. Tergitol NP-9 (a selectivity value of 2.9) was not as effective at inhibiting Renilla luciferase (Tergitol NP-9 is a sequestering agent of coelenterazine). This level of selectivity can be set as an indicator of selective quench from sequestering colenterazine. As other detergents were able to selectively quench Renilla luciferase-mediated luminescence much more efficiently, these detergents most likely have an effect on the Renilla luciferase enzyme rather than on sequestering of the enzyme.

TABLE 2

| | Retained | Retained | |
|---|---|---|---|
| | Firefly | Renilla | |
| | Luciferase | Luciferase | |
| | Luminescence | Luminescence | Selectivity |
| Detergent | (%) | (%) | Value |
| Anionic Detergents | | | |
| Sodium deoxycholate | 39.75 | 60.64 | 0.7 |
| Taurolithocholic acid | 81.32 | 75.03 | 1.1 |
| Zwitterionic Detergents | | | |
| CHAPSO | 70.07 | 52.15 | 1.3 |
| Zwittergent 3-10 | 73.92 | 33.45 | 2.2 |
| CHAPS | 73.84 | 47.29 | 1.6 |
| Nonionic Detergents | | | |
| Tergitol NP-9 | 79.16 | 27.14 | 2.9 |
| Cationic Detergents | | | |
| Benzalkonium | 0.20 | 0.04 | 5.0 |
| s-Acetylthiochloline bromide | 70.97 | 90.65 | 0.8 |
| (5-Bromopentyl) | 79.14 | 74.97 | 1.1 |
| trimethylammonium bromide | | | |
| Tetraphenylphosphonium | 90.86 | 24.49 | 3.7 |
| bromide | | | |
| Tetrapentylammounium | 80.89 | 37.01 | 2.2 |
| bromide | | | |
| Benzyldimethyldodecyl- | 20.67 | 0.08 | 259.9 |
| ammonium bromide (BDDABr) | | | |
| Trimethyl(2,4,5- | 0.06 | 1.87 | 0.0 |
| Trimethylbenzyl)ammonium | | | |
| bromide | | | |
| (3-Bromopropyl) | 72.20 | 71.58 | 1.0 |
| trimethylammonium bromide | | | |
| Cetyltrimethylammonium | 0.51 | 5.23 | 0.1 |
| chloride | | | |
| Hexamethonium bromide | 95.98 | 91.00 | 1.1 |
| (2-Bromoethyl) | 91.42 | 78.43 | 1.2 |
| trimethylammonium bromide | | | |
| Benzyldimethyltetradecyl- | 0.21 | 0.13 | 1.6 |
| ammonium bromide | | | |
| Cetyltrimethylammonium | 3.26 | 3.68 | 0.9 |
| chloride | | | |
| Decamthonium bromide | 85.42 | 76.68 | 1.1 |
| Dodecyltrimethylammonium | 42.07 | 1.74 | 24.2 |
| bromide | | | |
| Tetraoctylammonium | 43.66 | 43.12 | 1.0 |
| bromide | | | |

### Example 3

Additional detergents similar in structure to the highly effective selective quenching agents identified in Example 2 were screened for the ability to selectively quench Renilla luciferase-mediated luminescence. The assay was performed in the presence of Tergitol NP-9 to identify compounds that may affect Renilla luciferase rather than sequester coelenterazine. In addition to being a sequestering agent, Tergitol NP-9 is also a cell lysis detergent. Therefore, identified detergents are those that effectively quench in the presence of cell lysis detergents. Common structural features for successful quenchers in the presence of cell lysis detergents were the presence of a long carbon chain, phenyl groups, ammonium and bromide.

Each tested detergent (Table 3) was added to 0.1% (w/v) to a reagent solution containing 50 mM HEPES pH 7.4, 5 mM MgSO₄, 0.5 mM 1,2-diaminocyclohexanetetra-acetic acid (CDTA), 0.5% Tergitol NP-9, 30 mM thiourea, 1.58 mM ATP (Pharmacia/Invitrogen) and 0.1 mM 5′ fluoroluciferin (Promega Biosciences). Serial dilutions of 20% were made using reagent solutions which contained or did not contain the tested detergent. Just prior to the luciferase reaction, 0.1% Prionex® (Pentapharm) and 2.9 μM coelenterazine-h (Promega) were added to each solution. Unless otherwise specified, materials were obtained from Sigma-Aldrich.

The firefly luciferase and Renilla luciferase enzymes were diluted as follows. Firefly luciferase was diluted to 14 ng/ml in Delbecco's Minimal Essential (DMEM) medium (Gibco/Invitrogen) containing 0.1% Prionex®. Renilla luciferase was diluted to 10 ng/ml in DMEM medium containing 0.1% Prionex®.

The luciferase reactions were initiated by adding 50 μl of the firefly luciferase or Renilla luciferase enzyme solution to 50 μl of reagent solution. The reactions were performed as described above. The retained firefly luciferase and Renilla luciferase luminescence as well as the selectivity values for each quencher were calculated (Table 3). The values listed are based on 0.06% quencher in the reagent solution.

As the data in Table 3 shows, additional quenching agents were identified which selectively quenched Renilla luciferase luminescence (e.g., N-(biphenyl-2-ylmethyl)-N,N-dimethyldodecyan-1-ammonium bromide and N-dodecyl-N,N-dimethyl-9H-fluoren-9-aminium bromide) as well as or better than those in Example 1. The agents identified also had selectivity values greater than that of Tergitol NP-9, demonstrating that they may effect Renilla luciferase rather than sequester coelenterazine. It should be noted that benzyldodecyldimethylammonium bromide (BDDABr) was tested in Example 2. In the screen in this Example, the selectivity value for BDDABr was much lower (about 50-fold) than in Example 1. This is due to the presence of Tergitol NP-9 in the reagent solution. Tergitol NP-9 prevents BDDABr from inhibiting Renilla luciferase-mediated luminescence. The effect of detergents and cell lysis agents on Renilla luciferase quencher is further examined in Example 6.

TABLE 3

| | Retained | Retained | |
|---|---|---|---|
| | Firefly | Renilla | |
| | Luminescence | Luminescence | Selectivity |
| Detergent | (%) | (%) | Value |
| Benzyldimethyl(2- | 174.0 | 20.5 | 8.5 |
| dodecyloxethyl)ammonium | | | |
| chloride | | | |
| Dimethyldodecyl(5,6,7,8- | 112.0 | 118.0 | 0.9 |
| tetrahydro-2-naphthylmethyl) | | | |
| ammonium chloride | | | |
| 4-((Dodecyldimethylammonium) | 127.0 | 20.0 | 6.4 |
| methyl)benzoate (#1424) | | | |
| N-(biphenyl-2-ylmethyl)-NN- | 177.0 | 0.8 | 221.3 |
| dimethyldodecyan-1-ammonium | | | |
| bromide | | | |
| Benzyldodecyldimethyl | 119 | 22 | 5.4 |
| ammonium bromide (BDDABr) | | | |
| N-benzyl-N,N-dimethylloctan- | 134 | 13 | 10.3 |
| 1-aminium bromide | | | |
| N-benzyl-N,N-dimethylhexan- | 103 | 54 | 1.9 |
| 1-aminium bromide | | | |
| N-dodecyl-N,N-dimethyl-9H- | 37 | 1 | 37 |
| fluoren-9-aminium bromide | | | |
| N-benzyl-N,N- | 90 | 76 | 1.2 |
| dimethylcyclohexanaminium | | | |
| bromide | | | |

### Example 4

Neutral detergents, such as those present in cell lysis reagents, sequester inhibiting agents, thereby preventing them from inhibiting Renilla luciferase-mediated luminescence. This effect was seen in Examples 1 and 2 when a different selectivity value was obtained for BDDABr in the absence and presence of Tergitol NP-9. To determine if this effect was seen with other identified quenchers, Tergitol NP-9, a non-ionic detergent, and digitonin, a cell lysis agent with high critical micelle concentration (CMC), were tested with 6 cationic, quaternary ammonium detergent quenching agents described herein.

Renilla luciferase-mediated luminescence was measured from samples containing 9.6 mM phosphate buffer pH 6.8, 2.7 mM KCl, 137 mM NaCl (in PBS), 0.1% gelatin, 75 nM coelentrazine (Promega) and 10.2 ng/ml Renilla luciferase. Reactions contained either no lysing agent, 0.5% Tergitol NP-9 or 20 μg/ml digitonin. The detergents to be tested were titrated to 0.05%, 0.005% or 0.0005% in the reactions prior to the addition of the Renilla luciferase. Renilla luciferase-mediated luminescence was measured as described herein.

For measurement of firefly luciferase-mediated luminescence, a solution containing 110 mM HEPES pH 7.5, 8 mM MgSO₄, 0.05% gelatin, 7.35 ng/ml firefly luciferase (QuantiLum® Luciferase; Promega) and the content of the Steady-Glo Luciferase Substrate (Promega) at the equivalent of 1×, As in the Renilla luciferase reactions, reactions contained either no lysing agent, 0.5% Tergitol NP-9 or 20 μg/ml digitonin. The same detergents were titrated as described above and firefly luciferase-mediated luminescence was measured.

As the data shows (Table 4), the selective inhibition of the detergents tested was diminished in the presence of either Tergitol NP-9 or digitonin. Those quenching agents containing a benzyl group, a long chain carbon, or both, and demonstrated to selectively quench Renilla luciferase-mediated luminescence in the absence of neutral detergents, only moderately quench in the presence of such detergents. For example, in the absence of Tergitol NP-9, BDDABr and benzyldimethylteraceylammonium bromide (both containing a long carbon chain and benzyl group) are excellent selective quenchers (>1500-fold), but in the presence of Tergitol NP-9, the compounds were not as effective (<8-fold). As neutral detergents are often present for cell lysis, quenchers not affected by these detergents would be preferred as a Renilla luciferase luminescence quencher.

TABLE 4

| | | | Relative Renilla Luc | | | Relative Firefly Luc | | | Selective Inhibition | | |
|---|---|---|---|---|---|---|---|---|---|---|---|
| | | [Detergent] | Intensity | | | Intensity | | | (Ff Luc Inh/Ren Luc Inh) | | |
| Detergent | Description | (w:v) | Terg | Dig | Water | Terg | Dig | Water | Terg | Dig | Water |
| Cetyltrimethyl- | no benzyl | 0.0500% | 6% | 0.02% | 0.02% | 38% | 29% | 31% | 6 | 1395 | 1611 |
| ammonium chloride | group, has | 0.0050% | 71% | 0.02% | 0.02% | 94% | 65% | 70% | 1.3 | 2829 | 3848 |
| | long chain | 0.0005% | 95% | 14% | 13% | 98% | 164% | 179% | 1.0 | 12 | 13 |
| | (16C) | 0.0000% | 100% | 100% | 100% | 100% | 100% | 100% | 1.0 | 1.0 | 1.0 |
| Benzyldimethyldodecyl- | all parts, | 0.0500% | 2% | 0.02% | 0.02% | 50% | 37% | 79% | 21 | 1575 | 3627 |
| ammonium bromide | (12C) | 0.0050% | 58% | 5% | 4% | 101% | 139% | 155% | 1.7 | 29 | 36 |
| | | 0.0005% | 90% | 61% | 54% | 96% | 111% | 116% | 1.1 | 1.8 | 2.2 |
| | | 0.0000% | 100% | 100% | 100% | 100% | 100% | 100% | 1.0 | 1.0 | 1.0 |
| Benzyldimethyl- | all parts, | 0.0500% | 5% | 0.02% | 0.02% | 45% | 1.4% | 10% | 10 | 60 | 451 |
| tetradecylammonium | (14C) | 0.0050% | 70% | 0.03% | 0.01% | 93% | 128% | 106% | 1.3 | 5067 | 7068 |
| bromide | | 0.0005% | 96% | 14% | 12% | 98% | 155% | 126% | 1.0 | 11.0 | 10.2 |
| | | 0.0000% | 100% | 100% | 100% | 100% | 100% | 100% | 1.0 | 1.0 | 1.0 |
| Tetrapentylammonium | no benzyl | 0.0500% | 38% | 40% | 37% | 95% | 117% | 110% | 2.5 | 2.9 | 3.0 |
| bromide | group, no | 0.0050% | 81% | 87% | 83% | 96% | 104% | 101% | 1.2 | 1.2 | 1.2 |
| | long straight | 0.0005% | 96% | 99% | 98% | 97% | 100% | 98% | 1.0 | 1.0 | 1.0 |
| | chain | 0.0000% | 100% | 100% | 100% | 100% | 100% | 100% | 1.0 | 1.0 | 1.0 |
| Dodecyltrimethyl- | no benzyl | 0.0500% | 9% | 2% | 2% | 71% | 184% | 197% | 8 | 98 | 110 |
| ammonium bromide | group, has | 0.0050% | 76% | 55% | 51% | 93% | 128% | 128% | 1.2 | 2.3 | 2.5 |
| | long chain | 0.0005% | 98% | 96% | 95% | 96% | 103% | 107% | 1.0 | 1.1 | 1.1 |
| | (12C) | 0.0000% | 100% | 100% | 100% | 100% | 100% | 100% | 1.0 | 1.0 | 1.0 |
| Trimethyl(2,4,5Tri- | no long | 0.0500% | 4% | 0.03% | 0.03% | 12% | 1% | 1% | 2.9 | 47 | 50 |
| methylbenzyl) | chain, has | 0.0050% | 67% | 3% | 3% | 83% | 52% | 54% | 1.2 | 18 | 17 |
| ammonium bromide | benzyl | 0.0005% | 92% | 63% | 62% | 95% | 116% | 116% | 1.0 | 1.8 | 1.9 |
| | | 0.0000% | 100% | 100% | 100% | 100% | 100% | 100% | 1.0 | 1.0 | 1.0 |

### Example 5

Commercially available quaternary and ammonium/phosphonium detergents were also screened for the ability to selectively quench Renilla luciferase-mediated luminescence. The detergents screened were structurally similar to the quenching agents identified above in Examples 2-3. These detergents were also screened in the presence of Tergitol NP-9 to identify those that quench Renilla luciferase-mediated luminescence in the presence of cell lysis agents. Unless otherwise specified, materials were obtained from Sigma-Aldrich.

Each detergent was added at 0.2% to a reagent solution containing 50 mM Tricine pH 8.3, 3.5 mM MgSO₄, 1 mM CDTA, 30 mM thiourea and 0.5% Tergitol NP-9. This solution or one not containing a detergent inhibitor was used to resuspend the Bright-Glo™ luciferin substrate (Promega). Serial dilutions of 20% were made using reagent solutions which contained or did not contain the detergent. Prior to initiation of the luciferase reactions, 0.1% Prionex® (Pentapharm) and 2.9 μM coelentrazine-h (Promega) were added to each solution.

The firefly luciferase and Renilla luciferase enzymes were diluted as described in Example 3. The luciferase reactions were initiated by adding 100 μl of the firefly luciferase or Renilla luciferase enzyme solutions to 100 μl of reagent solution. The reactions were performed as described above. The retained firefly luciferase and Renilla luciferase luminescence as well as the selectivity values were calculated (Table 5). The detergent concentrations listed refer to the final concentration in the reaction.

As the data in Table 5 shows, additional quenching agents were identified which effectively and selectively quench Renilla luciferase-mediated luminescence. Generally, the best inhibitors had values >10, and those with moderate activity had values between 10 and 2. Structurally, carbons chains with less than about 10 atoms, or at least 1 to 2 atoms between a benzyl ring and a nitrogen, were present in inhibitors with high selectivity values.

TABLE 5

| | | Retained | Retained | |
|---|---|---|---|---|
| | | Firefly | Renilla | |
| | Final | Luciferase | Luciferase | |
| | Conc. | Luminescence | Luminescence | Selectivity |
| Detergent | (%) | (%) | (%) | Value |
| Benzyldimethyldodecyl- | 0.03 | 100 | 25 | 4 |
| ammonium bromide | 0.10 | 97 | 2 | 48.5 |
| (BDDABr) | | | | |
| Benzyldimethyldecyl- | 0.03 | 114 | 18 | 6.3 |
| ammonium chloride | 0.10 | 112 | 1 | 112 |
| Benzyldimethylhexa- | 0.03 | 100 | 47 | 2.1 |
| decylammonium chloride | 0.10 | 97 | 3 | 32.2 |
| (−)-N-dodecyl-N-methyl | 0.03 | 106 | 15 | 7.1 |
| ephedrium bromide | 0.10 | 102 | 1 | 102.1 |
| (1-(4-methoxy- | 0.03 | 106 | 20 | 5.3 |
| benzoyl)-undecyl)- | 0.10 | 104 | 2 | 52 |
| trimethylammonium | | | | |
| bromide | | | | |
| Benzyldimethylphenyl- | 0.03 | 82 | 38 | 2.2 |
| ammonium chloride | 0.10 | 55 | 12 | 4.6 |
| Benzyl-but-2-enyl- | 0.03 | 106 | 50 | 2.1 |
| diphenyl-phosphonium | 0.10 | 109 | 14 | 7.8 |
| bromide | | | | |
| (4-Penten-1- | 0.03 | 98 | 57 | 1.7 |
| yl)triphenylphos- | 0.10 | 102 | 21 | 4.9 |
| phonium bromide | | | | |
| (4-Methyl- | 0.03 | 99 | 65 | 1.5 |
| benzyl)tributyl- | 0.10 | 106 | 32 | 3.3 |
| phosphonium chloride | | | | |
| Benzyldiethyl(2,6- | 0.03 | 110 | 74 | 1.5 |
| xylycarbamoylethyl)- | 0.10 | 102 | 46 | 2.2 |
| ammonium benzoate | | | | |
| Benzyldimethylphenyl- | 0.03 | 101 | 78 | 1.3 |
| ammonium bromide | 0.10 | 107 | 81 | 1.3 |
| Dimethyl(4- | 0.03 | 99 | 78 | 1.3 |
| nitrobenzyl)phenyl- | 0.10 | 106 | 49 | 2.2 |
| phosphonium bromide | | | | |
| Isopropenylmethyldi- | 0.03 | 95 | 82 | 1.2 |
| phenylphosphonium | 0.10 | 109 | 51 | 2.1 |
| iodide | | | | |
| R424005 | 0.03 | 101 | 86 | 1.2 |
| | 0.10 | 101 | 81 | 1.2 |
| Benzyltriethyl- | 0.03 | 97 | 88 | 1.1 |
| phosphonium bromide | 0.10 | 102 | 51 | 2.0 |
| 1,1,2,2-Tetramethyl- | 0.03 | 87 | 88 | 1.0 |
| 1,2-dihydor- | 0.10 | 74 | 89 | 1.2 |
| quinolynium iodide | | | | |
| 1,1-Dimethyl-8- | 0.03 | 81 | 88 | 0.9 |
| hydroxy-1,2,3,4- | 0.10 | 68 | 82 | 0.8 |
| tetrahydroquinolium | | | | |
| iodide | | | | |
| Bezyltrimethyl- | 0.03 | 76 | 93 | 0.8 |
| ammonium iodide | 0.10 | 62 | 97 | 0.6 |
| Edrophonium chloride | 0.03 | 98 | 93 | 1.0 |
| | 0.10 | 102 | 89 | 1.1 |
| Benzyltriethyl- | 0.03 | 97 | 95 | 1.0 |
| ammonium bromide | 0.10 | 100 | 87 | 0.9 |
| Benzyltrimethyl- | 0.03 | 99 | 95 | 1.0 |
| ammonium bromide | 0.10 | 100 | 87 | 1.0 |
| (3-Carboxyl-propyl)- | 0.03 | 98 | 99 | 1.0 |
| methyl-diphenyl- | 0.10 | 102 | 89 | 0.9 |
| phosphonium chloride | | | | |

### Example 6

Non-detergent compounds were screened for the ability to selectively quench Renilla luciferase-mediated luminescence. Unless otherwise specified, materials were obtained from Sigma-Aldrich.

Each compound was diluted into 200 mM MOPS pH 6.7, 4 μM MgSO₄, 1 mM CDTA, 0.5% Tergitol NP-9, 0.05% Mazu-DF-204, 0.03% BDDABr, 30 mM thiourea, 1 mM bis-(2-mercaptoethylsulfone) (BMS), 4 μM CoA, and 1 μM 5-fluoroluciferin. Just prior to the luciferase reaction, 0.1% Prionex® (Pentapharm) and 2.9 μM coelenterazine-h (Promega) were added to each solution.

The firefly luciferase and Renilla luciferase enzymes were diluted as described in Examples 3 and 5. The luciferase reactions were initiated by adding 50 μl of the firefly luciferase and Renilla luciferase enzyme solution to 50 μl of reagent solution. The reactions were performed as described above. The retained Renilla and firefly luciferase-mediated luminescence and selectivity values for each inhibitor tested are listed in Table 6.

TABLE 6

| | | Retained | Retained | |
|---|---|---|---|---|
| | Inhibitor | Firefly | Renilla | |
| | Concentration | Luciferase | Luciferase | Selectivity |
| Inhibitor | (μM) | Value (%) | Value (%) | Value |
| Inhibitors with Selectiv- | | | | |
| ity at ≧10 at least one | | | | |
| tested concentration | | | | |
| 3077- | 1000 | 98 | 0.1 | 980 |
| (E)-N-(2-(5-(3-(4- | 100 | 98 | 1.0 | 98 |
| methoxybenzylamino)-3- | 10 | 100 | 5.0 | 20 |
| oxyprop-1-enyl)-1H-indol- | | | | |
| 3-yl)ethyl)-N,N- | | | | |
| dimethylbutan-1-aminium | | | | |
| iodide | | | | |
| 3078- | 1000 | 99 | 0.1 | 990 |
| (E)-N-(2-(5-(3-(4- | 100 | 100 | 1.0 | 100 |
| methoxybenzylamino)-3- | 10 | 100 | 5.0 | 20 |
| oxyprop-1-enyl)-1H-indol- | | | | |
| 3-yl)ethyl)-N,N- | | | | |
| dimethylhexan-1-aminium | | | | |
| iodide | | | | |
| 3051- | 1000 | 102 | 1.9 | 53.7 |
| (E)-N-(2,4- | 100 | 102 | 14 | 7.3 |
| dimethoxybenzyl)-3-(3-(2- | 10 | 102 | 55 | 1.9 |
| (dimethylamino)ethyl)-1H- | | | | |
| indol-5-yl)acrylamide | | | | |
| 3062- | 1000 | 99 | 5.1 | 19.4 |
| (E)-3-(3-(2- | 100 | 98 | 30 | 3.3 |
| (dimethylamino)ethyl-1H- | 10 | 100 | 74 | 1.4 |
| indol-5-yl)-N-(4- | | | | |
| methoxyphenethyl)acrylamide | | | | |
| 3063- | 1000 | 102 | 3.1 | 33 |
| (E)-3-(3-(2- | 100 | 102 | 19 | 5.4 |
| (dimethylamino)ethyl)-1H- | 10 | 102 | 63 | 1.6 |
| indol-5-yl)-N-(4- | | | | |
| nitrobenzyl)acrylamide | | | | |
| 3064- | | | | |
| (E)-3-(3-(2- | 1000 | 103 | 9.0 | 11.4 |
| (dimethylamino)ethyl)-1H- | 100 | 101 | 35 | 2.9 |
| indol-5-yl)-1- | 10 | 102 | 81 | 1.3 |
| morpholinoprop-2-en-1-one | | | | |
| 3067- | 1000 | 100 | 7.0 | 14.3 |
| (E)-3-(3- | 100 | 99 | 34 | 2.9 |
| ((dimethylamino)ethyl)-1H- | 10 | 100 | 79 | 1.3 |
| indol-5-yl)-N-(4- | | | | |
| methoxybenzyl)acrylamide | | | | |
| 3068- | 1000 | 102 | 2.1 | 48.6 |
| (E)-3-(3-(2- | 100 | 102 | 10 | 10.2 |
| dimethylamino)ethyl)-1H- | 10 | 101 | 40 | 2.5 |
| indol-5-yl)-N-(4- | | | | |
| methyloxybenzyl)acrylamide | | | | |
| 3070- | 1000 | 86 | 1.1 | 78.2 |
| (E)-N-(2-(5-(3-(4- | 100 | 96 | 6 | 16 |
| methoxybenzylamino)-3- | 10 | 99 | 30 | 3.3 |
| oxoprop-1-enyl)-1H-indol- | | | | |
| 3-yl)ethyl)-N,N- | | | | |
| dimethyloctan-1-aminium | | | | |
| iodide | | | | |
| 3071- | 1000 | 100 | 2.5 | 40 |
| (E)-3-(3-(3- | 100 | 101 | 15 | 6.7 |
| (dimethylamino)propyl)- | 10 | 101 | 57 | 1.8 |
| 1H-indol-5-yl)-N-(4- | | | | |
| methoxybenzyl)acrylamide | | | | |
| 3044- | 1000 | 132 | 7.6 | 18.9 |
| (E)-2-cyano-3-(3-(2- | 100 | 100 | 36 | 2.8 |
| (dimethylamino)ethyl)-1H- | 10 | 99 | 85 | 1.2 |
| indol-5-yl)-N-(4- | | | | |
| methoxybenzyl)acrylamide | | | | |
| Inhibitors with selectivity | | | | |
| at >2 at least one tested | | | | |
| concentration | | | | |
| 3046- | 1000 | 83 | 24.5 | 3.4 |
| (E)-N-(4- | 100 | 99 | 78 | 1.3 |
| methanoxybenzyl)-3- | 10 | 100 | 96 | 1.0 |
| (quinolin-6-yl)acrylamide | | | | |
| 3055- | 1000 | 99 | 20 | 5.0 |
| (E)-3-(1-benzyl-3-(2- | 100 | 98 | 44 | 2.2 |
| (dimethylamino)ethyl)-1H- | 10 | 100 | 85 | 1.2 |
| indol-5-yl)-N-(4- | | | | |
| methoxybenzyl)acrylamide | | | | |
| 3056- | 1000 | 89 | 32.6 | 2.7 |
| (E)-3-(1H-indol-5-yl)-N-(4- | 100 | 100 | 79 | 1.3 |
| methyloxybenzyl)acrylamide) | 10 | 101 | 93 | 1.1 |
| 3061- | 1000 | 82 | 36 | 2.3 |
| (E)-6-(3-(4- | 100 | 100 | 85 | 1.8 |
| methoxybenxylamino)-3- | 10 | 103 | 96 | 1.1 |
| oxoprop-1-enyl)-1- | | | | |
| methylquinolinium iodide | | | | |
| 3073- | 1000 | 101 | 34.5 | 2.9 |
| (E)-3-(3-(2- | 100 | 100 | 87 | 1.5 |
| (dimethylamino)ethyl)-1H- | 10 | 101 | 102 | 0.99 |
| indol-5-yl)-N-(2,4,6- | | | | |
| trimethoxybenzyl)acrylamide | | | | |
| Inhibitors with Selectivity <2 | | | | |
| at all tested concentrations | | | | |
| 3072- | 1000 | 101 | 55.2 | 1.8 |
| (E)-3-(3-(2- | 100 | 101 | 95 | 1.1 |
| (dimethylamino)ethyl)-1H- | 10 | 101 | 101 | 1 |
| indol-5-yl)-N-(3,4,5-tri- | | | | |
| methyoxybenzyl)acrylamide | | | | |
| 3031- | 1000 | 89 | 66.5 | 1.4 |
| 2-(3-(2- | 153 | 89 | 90 | 0.99 |
| (dimethylamino)ethyl)-1H- | 23 | 96 | 96 | 1 |
| indol-5-yloxy)-N-(4- | | | | |
| metholybenzyl)acetamide | | | | |
| 3045- | 1000 | 98 | 74.7 | 1.3 |
| (E)-4-methoxybenzyl-3-(3- | 153 | 100 | 98 | 1.0 |
| (2-(dimethylamino)ethyl)- | 23 | 100 | 97 | 1.0 |
| 1H-indol-5-yl)acrylate | | | | |
| 3050- | 1000 | 102 | 55 | 1.9 |
| (E)-3-(3-(2- | 153 | 102 | 99 | 1.0 |
| (dimethylamino)ethyl)-1H- | 23 | 102 | 93 | 1.1 |
| indol-5-yl)-N- | | | | |
| hexylacrylamide | | | | |
| 3053- | 1000 | 91 | 84.7 | 1.1 |
| N-(4-methoxybenzyl)-2- | 153 | 97 | 90 | 1.1 |
| (quinolin-6-yl)acrylamide | 23 | 100 | 95 | 1.1 |
| 3054- | 1000 | 101 | 93.3 | 1.1 |
| 2-(1H-indol-5-yl)-N-(4- | 153 | 102 | 95 | 1.1 |
| methoxybenzyl)acrylamide | 23 | 101 | 95 | 1.1 |
| 3057- | 1000 | 102 | 77.5 | 1.3 |
| (E)-N-(4-methoxybenzyl)- | 153 | 98 | 93 | 1.1 |
| 3-(4-methyl-2-oxo-2H- | 23 | 100 | 100 | 1 |
| chromen-6-yl)acrylamide | | | | |
| 3058- | 1000 | 103 | 98.8 | 1.04 |
| N-(4-methoxybenzyl)-2-(4- | 153 | 102 | 98 | 1.04 |
| methyl-2-oxo-2H-chromen- | 23 | 101 | 96 | 1.05 |
| 6-yl)acrylamide | | | | |
| 3059- | 1000 | 101 | 94.6 | 1.07 |
| (E)-10-bromo-2-cyano-N- | 153 | 101 | 96 | 1.05 |
| (4-methoxybenzyl)dec-2- | 23 | 101 | 99 | 1.02 |
| enamide | | | | |

### Example 7

In Example 6, non-detergent compounds were identified which selectively quenched Renilla luciferase-mediated luminescence. The compounds were screened in the presence of a previously identified detergent quencher, BDDABr. To determine whether the effect of the combination of detergent and non-detergent quenching agents was additive, independent (i.e., the effect of both is greater than the sum of each) or overshadowed by the other quenching agent, the level of inhibition was determined for one of the quaternary amide detergent inhibitors, 4-((dodecyldimethylammonium)methyl)benzoate (#1424), and one of the indoles, compound #3077. The compounds were tested separately and in combination using lysates derived from cells expressing Renilla luciferase.

Renilla luciferase was expressed under the control of the CMV promoter in Chinese Hamster Ovary (CHO) cells. The cells were grown in DMEM+10% FBS (Invitrogen). The cells (20,000) were plated in 96-well plates and cultured in the presence of the EnduRen™ Live Cell Substrate (Promega) in order to generate continuous Renilla luciferase-mediated luminescence. After 12 hours of culturing, Renilla luciferase-mediated luminescence was measured. After measurement, the cells were lysed with an equal volume of a solution containing 0.5% Tergitol NP-9 and either 10 μM compound #3077 (non-detergent inhibitor), 0.04% 4-((dodecyldimethylammonium)methyl)benzoate (#1424) (detergent inhibitor), or a combination of the two. The cells were exposed for 3 minutes and Renilla luciferase-mediated luminescence was measured.

As the data in Table 7 shows, the effect of a combination of the two inhibitors exceeds the effect of each compound demonstrating that the agents are quenching independently of each other. The data also demonstrates that compound #3077 alone is more effective (about 12-fold) at quenching Renilla luciferase-mediated luminescence than 4-((Dodecyldimethylammonium)methyl)benzoate (#1424) alone. Also, the example demonstrates that the two quenchers are effective at quenching in the presence of Tergitol NP-9 Renilla luciferase luminescence.

TABLE 7

| | | Renilla | |
|---|---|---|---|
| | Initial | luciferase | |
| | Renilla | luminescence | |
| | luciferase | after inhibitor | Inhibition |
| | luminescence | addition | (Initial/Addition) |
| No Inhibitor Added | 1012320 | 178073 | 6 |
| Compound #3077 Added | 852596 | 1402 | 608 |
| ((Dodecyldimethylammonium) | 1037560 | 19963 | 52 |
| methyl)benzoate | | | |
| Compound #3077 & | 854084 | 682 | 1252 |
| ((Dodecyldimethylammonium) | | | |
| methyl)benzoate | | | |

### Example 8

In this example, the necessity of selective Renilla luciferase-mediated luminescence quenchers in dual reporter assay in which Renilla luminescence is first measured is demonstrated. Often in dual reporter assays, Renilla luciferase is used as an internal control for the normalization of gene expression. Normalization is a method by which data are corrected for factors (e.g., cell number and transfection efficiency) other than those being directly tested in an experiment. For example, the reporter activity from firefly luciferase (containing test sequence) in a sample is divided by the reporter activity from Renilla luciferase in the same sample. In this example, a dual reporter assay was performed in which Renilla luminescence was measured twice prior to the measurement of firefly luminescence. The two Renilla luminescence measurements taken included an initial measurement followed by a measurement (residual luminescence) after the addition (or not) of Renilla luciferase luminescence quenching agents. The resulting residual luminescence seen in the absence of the quenching agents demonstrate the contribution of Renilla luminescence to the firefly luminescence measurement causing the quantitation of gene expression from firefly luciferase to be unreliable.

Renilla luciferase and firefly luciferase were co-expressed in CHO cells as described in Example 7. A CMV promoter was employed to express Renilla luciferase and a SV40 promoter was employed to express firefly luciferase. After a 5 hour incubation in the presence of 60 μM EnduRen™ (Promega), an initial Renilla luciferase-mediated luminescence measurement was taken. After Renilla luminescence was measured, the cells were lysed in 0.1% Tergitol NP-9. To half of the wells, 0.2 mM compound #3077 and 0.02% ((dodecyldimethylammonium)methyl)benzoate (final concentration) were added to quench Renilla luciferase-mediated luminescence. The other wells served as the “no quenching agents added” controls. After a 3 minute incubation, the Renilla luciferase-mediated luminescence was again measured to obtain a “residual” Renilla luciferase luminescence measurement. For the measurement of firefly luminescence, a firefly luciferase reagent (final concentration: 25 mM HEPES pH 7.5, 8 mM MgSO₄, 0.5 mM ATP, 5 mM DTT and 0.5 mM luciferin) was added to all wells. Firefly luciferase-mediated luminescence was then measured as previously described.

As the data in Table 8 indicates, in the absence of the Renilla luciferase quenching agents, the residual Renilla luciferase luminescence remains high, thereby contributing to the measured firefly luciferase luminescence. In the absence of the quenching agents, it is necessary to subtract the residual Renilla luciferase-mediated luminescence from the measured firefly luciferase-mediated luminescence to effectively quantify expression from firefly luciferase-mediated reactions. In the presence of the quenching agents, the residual Renilla luciferase-mediated luminescence is minimal (less than 0.009% of the measured firefly luciferase-mediated luminescence). Therefore, subtraction and measurement of the residual Renilla luciferase-mediated luminescence is not necessary to obtain an accurate measurement of firefly luciferase expression in the presence of Renilla luciferase quenchers. This eliminates the need to measure residual Renilla luciferase luminescence to accurately quantify firefly luciferase expression allowing the development of a homogenous dual reporter gene assay in which Renilla luciferase luminescence is measured and quenched prior to measurement of firefly luciferase luminescence.

TABLE 8

| | | | | Adjusted firefly |
|---|---|---|---|---|
| | | Residual | Measured | luciferase |
| | Initial Renilla | Renilla | Firefly (FF) | luminescence |
| | luciferase | luciferase | luciferase | (Measured FF- |
| | luminescence | luminescence | luminescence | residual Renilla) |
| No Renilla | 851617 | 431965 | 1265656 | 833690 |
| Quenching | | | | |
| Agents | | | | |
| With Renilla | 805644 | 733 | 847627 | 846894 |
| Quenching | | | | |
| Agents | | | | |

### Example 9

This example demonstrates that the Renilla luciferase-mediated luminescence quenchers described herein can be used in a reagent also including cell lysis reagents and components necessary to measure firefly luciferase-mediated luminescence. This homogenous dual reporter gene assay measures and quenches Renilla luciferase luminescence prior to measurement of firefly luciferase luminescence.

HEK 293 cells stably expressing firefly luciferase under the control of the cAMP response element (CRE), dopamine D1 receptor under the control of the CMV promoter and a Renilla luciferase-neomycin fusion protein under the control of the SV40 promoter, were plated at 3300 cells/well in DMEM (Invitrogen) with 10% FBS (Hyclone) and 6 μM EnduRen™ Live Cell Substrate (Promega) into 12 wells of a 96-well plate. To half the wells, 10 μM dopamine was also added to induce firefly luciferase expression by stimulating CRE. After a 5 hour incubation, Renilla luciferase was measured using a VarioSkan Flash luminometer (ThermoFisher). A reagent containing Renilla luciferase luminescence quenchers, cell lysis agents and components necessary to measure firefly luciferase was added at 1:1 to all wells. The reagent comprised 100 mM PIPES pH 6.7, 20 mM MgSO₄, 9 mM CaCl₂, 1 mM CDTA, 0.5% Tergitol NP-9, 0.5% Mazu DF 204, 28 mM thiourea, 55.5 μM Napthol Yellow, 0.4% ammonium carboxylate (#1424), 300 μM indole (#3077), 1 mM TCEP (3,3′, 3″-phosphinidynetrispropanic acid hydrochloride; Promega Biosciences), 4 mM Coenzyme A, 6 mM ATP and 5 mM 5′-fluoroluciferin. The reagent and cells were incubated for 10 minutes to quench Renilla luciferase luminescence and lyse the cells. Firefly luminescence was then measured as previously described.

TABLE 9

| | Renilla | Firefly | | |
|---|---|---|---|---|
| | luciferase | luciferase | | Normalized |
| | luminescence | luminescence | Normalization | Fold |
| Sample | (RLU) | (RLU) | (Firefly/Renilla) | Induction |
| Uninduced | 8624 | 10422 | 1.2 | 1 |
| Induced | 7456 | 724,148 | 97.1 | 81 |

As is seen in Table 8, stimulation of resulted in an 81-fold induction of firefly luciferase expression. Therefore, the Renilla luciferase quenchers described herein can be added to a reagent comprising cell lysis agents and components necessary for firefly luciferase luminescence measurement to create a single reagent capable of quenching Renilla luciferase luminescence, cell lysis and measurement of firefly luciferase luminescence. This provides a homogenous dual reporter gene assay in which Renilla luciferase luminescence is measured then quenched prior to the measurement of firefly luciferase luminescence.

### Example 10

In this example, the broad applicability of the Renilla luciferase quenchers presented herein is demonstrated. Various versions of firefly luciferase reagents were tested to demonstrate that regardless of the firefly luciferase reagent used, the Renilla luciferase quenchers are effective at quenching Renilla luciferase without affecting firefly luciferase luminescence.

Eight different versions of firefly luciferase reagent were made. The versions contained one of two luciferin substrates: luciferin or 5′ fluoroluciferin. The versions also differed in whether they contained Coenzyme A and/or DTT (a thiol). All versions of the reagent contained 250 mM HEPES pH 7.4, 16 mM MgSO₄, 2 mM ATP and 2 mM luciferin or 5′fluoroluciferin. To create the various versions of the firefly luciferase reagent, 20 mM DTT, 2 mM Coenzyme A, 20 mM DTT and 2 mM Coenzyme A or neither DTT or Coenzyme A was added. In addition, sub-versions of the reagent were created which contained 0.3 mM of the indole (#3077) Renilla luciferase quencher, 0.04% (w:v) of the detergent (4-((dodecyldimethylammonium)methyl)benzoate) Renilla luciferase quencher, both quenchers or neither quencher. In all, a total of 32 versions of firefly luciferase reagent were created (Table 10).

To mimic the effect of measuring Renilla luciferase luminescence followed by the measurement of firefly luciferase luminescence in a single sample, side-by-side samples (each in triplicate) were used for each version of reagent. The first triplicate of samples comprised one of the versions of firefly luciferase reagent and a firefly luciferase solution (added 1:1) containing F12 medium (Invitrogen), 1 mg/ml BSA (ThermoFisher) and 14 ng/ml QuantiLum® luciferase (Promega). The second triplicate of samples comprised the same version of firefly luciferase reagent used in the first trio and a firefly/Renilla luciferase solution (added 1:1) containing F12 medium, 1 mg/ml BSA, 14 ng/ml QuantiLum® luciferase and 140 ng/ml Renilla luciferase. Firefly and Renilla luciferase luminescence was measured as previously described in both sets of samples at the same time. The results are displayed in Table 10. The firefly luciferase measurements were obtained from the samples only containing the firefly luciferase while the Renilla luciferase measurements were obtained from the samples containing both firefly and Renilla luciferases.

In the results in Table 10, the measurements from the firefly luciferase only samples demonstrate how much firefly luciferase luminescence should be seen in a sample. The Renilla luciferase luminescence measurement from the fireflylRenilla samples demonstrates how much the Renilla luciferase luminescence would obscure the firefly luciferase luminescence if the Renilla luciferase quenchers were not added. As seen in the results, the Renilla luciferase luminescence was at least 40-fold higher than the firefly luciferase luminescence in all the sample which did not contain any Renilla luciferase quenchers. Therefore, in the absence of the quenchers, firefly luciferase luminescence would not be measured even though it was present in the samples. When the Renilla luciferase quencher(s) were added, Renilla luciferase luminescence is quenched permitting the firefly luciferase luminescence to be measured. This example also demonstrates the utility of the Renilla luciferase quenchers in various versions of firefly luciferase reagent. The quenchers do not have an affect on the components present in the reagent and may be used with a variety of luciferin derivatives allowing firefly luciferase luminescence to be accurately measured in a variety of luminescent assays.

TABLE 10

| | Renilla | Luciferin | | 5′-fluoroluciferin | |
|---|---|---|---|---|---|
| | inhibitors | Firefly | Renilla | Firefly | Renilla |
| no | no add | 115,295 | 5,766,913 | 238,934 | 10,076,203 |
| thiol | 1424 | 144,239 | 660,916 | 268,289 | 1,138,027 |
| | 3077 | 115,749 | 136,759 | 239,138 | 280,188 |
| | both | 139,708 | 160,195 | 256,590 | 294,078 |
| DTT | no add | 265,576 | 5,281,970 | 523,809 | 7,846,683 |
| | 1424 | 291,173 | 699,059 | 512,625 | 1,142,573 |
| | 3077 | 252,225 | 260,392 | 499,900 | 514,808 |
| | both | 280,818 | 284,888 | 482,197 | 503,620 |
| CoA | no add | 1,545,567 | 8,388,327 | 2,401,880 | 11,668,900 |
| | 1424 | 1,621,883 | 2,240,063 | 2,095,790 | 2,891,880 |
| | 3077 | 1,502,143 | 1,572,983 | 2,367,927 | 2,360,350 |
| | both | 1,577,053 | 1,559,307 | 2,015,060 | 2,005,497 |
| DTT + | no add | 1,917,303 | 7,140,900 | 2,789,857 | 9,451,593 |
| CoA | 1424 | 1,881,453 | 2,265,505 | 2,376,253 | 2,833,435 |
| | 3077 | 1,821,797 | 1,770,665 | 2,677,367 | 2,653,610 |
| | both | 1,775,853 | 1,723,233 | 2,229,713 | 2,185,883 |

### Example 11

### General Indole Acrylate Syntheses

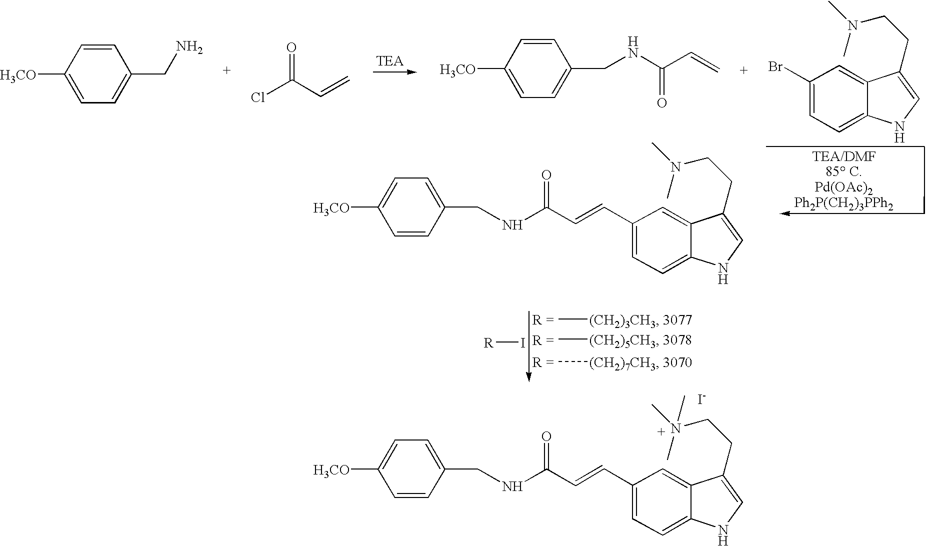

One class of Renilla inhibitors can be prepared as illustrated in the scheme above. These steps are readily amenable for scale-up and many variations can be carried out to prepare various compounds of the invention.

In the scheme above, 4-methoxybenzyl amine reacted with acryloyl chloride to form the corresponding amide. 5-Bromo-3-N,N-dimethylethanamine indole was prepared by literature procedures. An intermolecular Heck reaction between these two intermediates afforded a scaffold for many molecules of the invention, according to various embodiments. Finally, alkylation using the corresponding alkyl halides yielded the targeted quaternary ammonium salts.

### Example 12

### Synthesis of an Indole Acrylate, According to an Embodiment of the Invention

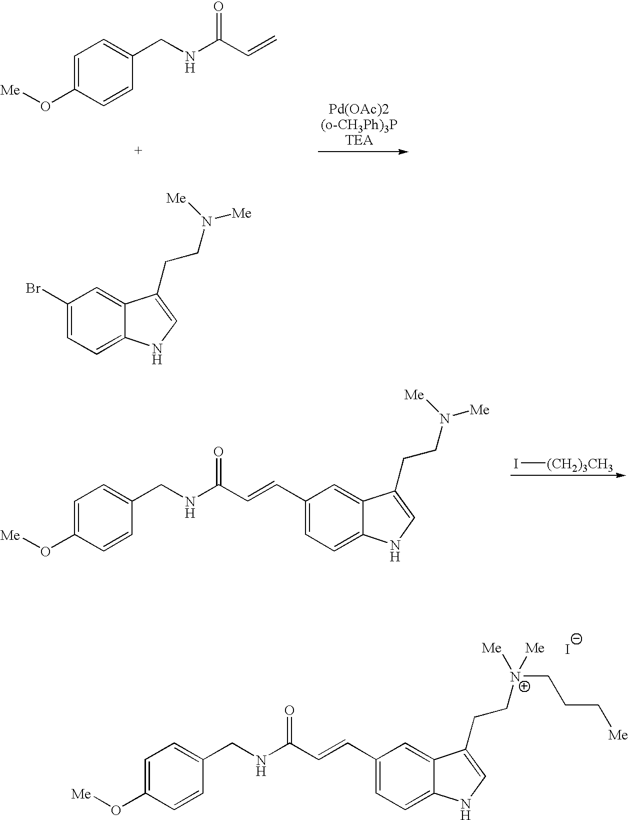

Synthesis of (E)-3-(3-(2-(dimethylamino)ethyl)-1H-indol-5-yl)-N-(4-methoxybenzyl)acrylamide. To a solution of N-(4-methoxybenzyl)acrylamide (0.77 g, 4 mmol) and 2-(5-bromo-1H-indol-3-yl)-N,N-dimethylethanamine (0.5 g, 2.9 mmol, (see J. Org. Chem. 1994, 59, 3738) in 5 mL of DMF were added Pd(OAc)₂ (0.09 g, 0.4 mmol), tri(o-toluene)phosphine (0.17 g, 0.8 mmol) and Et₃N (5 mL). The resultant mixture was heated to 100° C. for 3 hours. Upon cooling to ambient temperature (about 23° C.), 20 mL of ethyl acetate was added and an insoluble solid was removed by filtration. After removal of the solvent from the filtrate, the compound was purified by flash chromatography using methylene chloride/methanol as eluent.

Synthesis of (E)-N-(2-(5-(3-(4-methoxybenzylamino)-3-oxoprop-1-enyl)-1H-indol-3-yl)ethyl)-N,N-dimethylbutan-1-aminium iodide. The solution of (E)-3-(3-(2-(dimethylamino)ethyl)-1H-indol-5-yl)-N-(4-methoxybenzyl)acrylamide (0.20 g, 0.53 mmol) in 10 mL of ethanol was stirred with K₂CO₃ (0.15 g, 1.08 mmol) for 10 minutes. After removal of K₂CO₃, iodobutane (0.117 g, 0.64 mmol) was added to the filtrate. The solution was refluxed overnight. After removal of solvent, the desired compound was purified by flash chromatography using methylene chloride/methanol as eluent.

### REFERENCES

Annaert et al., Pharmaceut. Res., 14, 492 (1997).
Blaise et al., BioTechniques, 16, 932 (1994).
Bronstein, et al., Anal-Biochem., 219, 169 (1994).
Bronstein, et al., Bioluminescence and Chemiluminescence: Current Status. (eds. P. E. Stanley and L. J. Kricka) John Wiley & Sons, Inc. pp. 73-82 (1991).
Denburg et al., Archives of Biochemistry and Biophysics, 134, 381 (1969).
Denburg et al., Archives of Biochemistry and Biophysics, 141, 668 (1970).
Flanagan et al., J. Virology, 65, 769 (1991).
Inoue et al., Tetrahedron Letters, 31, 2685 (1977).
Jain et al., BioTechniques, 12, 681 (1992).
Kobatake et al., Bioluminescence and Chemiluminescence (ed. A. A. Szalay, et al.) John Wiley & Sons, Chichester, pp. 337-341 (1993).
Kondepudi et al., Abstract #725, Annual Meeting of the American Society of Cell Biologist, Dec. 10-14, 1994, San Francisco, Calif.
Leckie et al., BioTechniques, 17, 52 (1994).
Lee et al., Archives of Biochemistry and Biophysics, 141, 38-52 (1970).
Mathews et al., Biochemistry, 16, 85 (1977).
Redden et al., Int. J. Pharm., 180, 151 (1999).
Schaap et al., Clinical Chemistry, 35, 1863 (1989).
Schram, Bioluminescence and Chemiluminescence: Current Status. (eds. P. E. Stanley and L. J. Kricka) John Wiley & Sons, Inc., pp. 407-412 (1991).
Thompson et al., Gene, 103, 171 (1991).
Thorp et al., Methods in Enzymology, 133, 331 (1986).
Tsien, Nature, 290, 527 (1981).
U.S. Pat. No. 5,831,102.
Ward, Chemi- and Bioluminescence (ed. John Burr) Marcel Dekker, Inc., New York, pp. 321-358 (1985).
Wood, Curr. Op. Biotech., 6, 50 (1995).
Wood, in Bioluminescence & Chemiluminescence: Current Status. (eds. Stanley, P. E., and Kricka, J.) John Wiley & Sons, Chichester. pp. 543-546 (1991).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The claims are as follows:

1. A method of assaying an enzyme-mediated luminescence reaction comprising:
(a) detecting or determining luminescence energy produced by an anthozoan luciferase-, copepod luciferase-, or decapod luciferase-mediated luminescence reaction; and
(b) quenching photon emission from the anthozoan luciferase-, copepod luciferase-, or decapod luciferase-mediated luminescence reaction by introducing a composition comprising a compound of formula (I): 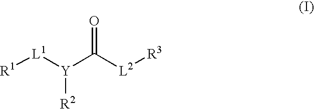
wherein
Y is N or O;
L¹ is (C₁-C₆)alkylene or a direct bond;
R¹ is alkyl, aryl, heteroaryl, or heterocycle;
R² is H, (C₁-C₆)alkyl, or absent;
or L¹ is a direct bond and R¹ and R² together with the nitrogen attached to R² form a heteroaryl or heterocycle group;
L² is optionally unsaturated straight chain or branched (C₁-C₆)alkylene or (C₁-C₆)alkylene-O—;
R³ is alkyl, aryl, heteroaryl, heterocycle;
wherein any alkyl, alkylene, aryl, heteroaryl, or heterocycle is optionally substituted with one to five alkyl, alkenyl, alkoxy, alkanoyl, alkanoyloxy, alkoxycarbonyl, cycloalkyl, cyano, nitro, halo, hydroxy, mercapto, oxo, —SOₙR⁴, N(R^{X})(R^{Y}), N(R^{X})(R^{Y})alkyl, N⁺(R^{X})(R^{Y})(R^{Z}), or N⁺(R^{X})(R^{Y})(R^{Z})alkyl groups wherein R^{X}, R^{Y}, and R^{Z} are each independently H, alkyl, aryl, heteroaryl, or heterocycle; and each substituent is optionally substituted with one to three R³ groups; and any nitrogen atom of a nitrogen heterocycle is optionally substituted with an optionally substituted alkyl or acyl group, or with a nitrogen protecting group;
n is 0, 1, 2, or 3; and
R⁴ is H, alkyl, or aryl;
or a salt thereof;
or a compound of formula (IV): 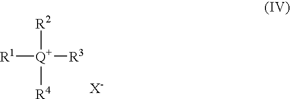
wherein
Q is N or P;
R¹ and R² are each independently alkyl, cycloalkyl, aryl, heteroaryl, heterocycle, arylalkyl;
R³ and R⁴ are each independently (C₁-C₆)alkyl; and
X is an organic or inorganic counterion;
or a combination thereof, to the luminescence reaction.

2. The method of claim 1 in which the composition further comprises reagents capable of initiating a second enzyme-mediated luminescence reaction distinct from the anthozoan luciferase-, copepod luciferase-, or decapod luciferase-mediated luminescence reaction; and
(c) detecting or determining luminescence energy produced by the second enzyme-mediated luminescence reaction.

3. The method of claim 1 wherein prior to detecting or determining luminescence energy a distinct second enzyme mediated luminescence reaction is initiated.

4. The method of claim 3 wherein the anthozoan luciferase-, copepod luciferase-, or decapod luciferase-mediated reaction is initiated at the same time as the distinct second enzyme-mediated luminescence reaction.

5. A method of assaying an enzyme-mediated luminescence reaction comprising:
(a) detecting or determining luminescence energy produced by an anthozoan luciferase-, copepod luciferase-, or decapod luciferase-mediated luminescence reaction; and
(b) introducing a composition capable of selectively quenching the anthozoan luciferase-, copepod luciferase-, or decapod luciferase-mediated luminescence reaction and initiating a second enzyme-mediated luminescence reaction distinct from the anthozoan luciferase-, copepod luciferase-, or decapod luciferase-mediated luminescence reaction, wherein the composition comprises a compound of formula (I): 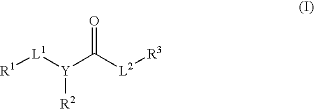
wherein
Y is N or O;
L¹ is (C₁-C₆)alkylene or a direct bond;
R¹ is alkyl, aryl, heteroaryl, or heterocycle;
R² is H, (C₁-C₆)alkyl, or absent;
or L¹ is a direct bond and R¹ and R² together with the nitrogen attached to R² form a heteroaryl or heterocycle group;
L² is optionally unsaturated straight chain or branched (C₁-C₆)alkylene or (C₁-C₆)alkylene-O—;
R³ is alkyl, aryl, heteroaryl, heterocycle;
wherein any alkyl, alkylene, aryl, heteroaryl, or heterocycle is optionally substituted with one to five alkyl, alkenyl, alkoxy, alkanoyl, alkanoyloxy, alkoxycarbonyl, cycloalkyl, cyano, nitro, halo, hydroxy, mercapto, oxo, —SOₙR⁴, N(R^{X})(R^{Y}), N(R^{X})(R^{Y})alkyl, N⁺(R^{X})(R^{Y})(R^{Z}), or N⁺(R^{X})(R^{Y})(R^{Z})alkyl groups wherein R^{X}, R^{Y}, and R^{Z} are each independently H, alkyl, aryl, heteroaryl, or heterocycle; and each substituent is optionally substituted with one to three R³ groups; and any nitrogen atom of a nitrogen heterocycle is optionally substituted with an optionally substituted alkyl or acyl group, or with a nitrogen protecting group;
n is 0, 1, 2, or 3; and
R⁴ is H, alkyl, or aryl;
or a salt thereof;
or a compound of formula (IV): 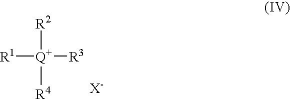
wherein
Q is N or P;
R¹ and R² are each independently alkyl, cycloalkyl, aryl, heteroaryl, heterocycle, arylalkyl;
R³ and R⁴ are each independently (C₁-C₆)alkyl; and
X is an organic or inorganic counterion;
or a combination thereof; and
(c) detecting or determining luminescence energy produced by the second enzyme-mediated luminescence reaction.

6. A method of assaying an enzyme-mediated luminescence reaction comprising:
(a) detecting or determining luminescence energy produced by an anthozoan luciferase-, copepod luciferase-, or decapod luciferase-mediated luminescence reaction;
(b) quenching photon emission from the anthozoan luciferase-, copepod luciferase-, or decapod luciferase-mediated luminescence reaction by introducing a composition comprising a compound of formula (I): 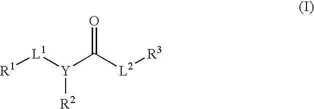
wherein
Y is N or O;
L¹ is (C₁-C₆)alkylene or a direct bond;
R¹ is alkyl, aryl, heteroaryl, or heterocycle;
R² is H, (C₁-C₆)alkyl, or absent;
or L¹ is a direct bond and R¹ and R² together with the nitrogen attached to R² form a heteroaryl or heterocycle group;
L² is optionally unsaturated straight chain or branched (C₁-C₆)alkylene or (C₁-C₆)alkylene-O—;
R³ is alkyl, aryl, heteroaryl, heterocycle;
wherein any alkyl, alkylene, aryl, heteroaryl, or heterocycle is optionally substituted with one to five alkyl, alkenyl, alkoxy, alkanoyl, alkanoyloxy, alkoxycarbonyl, cycloalkyl, cyano, nitro, halo, hydroxy, mercapto, oxo, —SOₙR⁴, N(R^{X})(R^{Y}), N(R^{X})(R^{Y})alkyl, N⁺(R^{X})(R^{Y})(R^{Z}), or N⁺(R^{X})(R^{Y})(R^{Z})alkyl groups wherein R^{X}, R^{Y}, and R^{Z} are each independently H, alkyl, aryl, heteroaryl, or heterocycle; and each substituent is optionally substituted with one to three R³ groups; and any nitrogen atom of a nitrogen heterocycle is optionally substituted with an optionally substituted alkyl or acyl group, or with a nitrogen protecting group;
n is 0, 1, 2, or 3; and
R⁴ is H, alkyl, or aryl;
or a salt thereof;
or a compound of formula (IV): 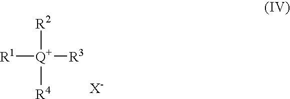
wherein
Q is N or P;
R¹ and R² are each independently alkyl, cycloalkyl, aryl, heteroaryl, heterocycle, arylalkyl;
R³ and R⁴ are each independently (C₁-C₆)alkyl; and
X is an organic or inorganic counterion;
or a combination thereof, to the luminescence reaction;
(c) introducing a composition capable of initiating a second enzyme-mediated luminescence reaction distinct from the anthozoan luciferase-, copepod luciferase-, or decapod luciferase-mediated luminescence reaction; and
(d) detecting or determining luminescence energy produced by the second enzyme-mediated luminescence reaction.

7. The method of claim 1, 5 or 6 wherein in step (a), a Renilla luciferase-mediated luminescence reaction is detected or determined.

8. The method of claim 2, 3, 4, 5 or 6 wherein the second enzyme-mediated luminescence reaction is mediated by a beetle luciferase.

9. A compound of formula (II): 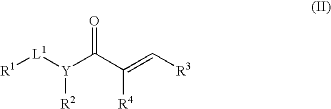
wherein
L¹ is (C₁-C₆)alkylene or a direct bond;
R¹ is alkyl, aryl, heteroaryl, or heterocycle;
R² is H, (C₁-C₆)alkyl, or absent;
or L¹ is a direct bond and R¹ and R² together with the nitrogen attached to R² form a heteroaryl or heterocycle group;
R³ is heteroaryl substituted with at least one quaternary ammonium group, heteroaryl comprising at least one quaternary amine-containing substituent, or alkyl substituted with at least one quaternary ammonium group;
R⁴ is H or CN;
wherein any alkyl, alkylene, aryl, heteroaryl, or heterocycle is optionally substituted with one to five alkyl, alkenyl, alkoxy, alkanoyl, alkanoyloxy, alkoxycarbonyl, cycloalkyl, cyano, nitro, halo, hydroxy, mercapto, oxo, —SOₙR⁴, N(R^{x})(R^{Y}), N(R^{x})(R^{Y})alkyl, N⁺(R^{X})(R^{Y})(R^{Z}), or N⁺(R^{X})(R^{Y})(R^{Z})alkyl groups wherein R^{X}, R^{Y}, and R^{Z} are each independently H, alkyl, aryl, heteroaryl, or heterocycle; each substituent is optionally substituted with one to three R³ groups; and any nitrogen atom of a nitrogen heterocycle is optionally substituted with an optionally substituted alkyl or acyl group, or with a nitrogen protecting group;
n is 0, 1, 2, or 3; and
R⁴ is H, alkyl, or aryl;
or a salt thereof.

10. The compound of claim 9 wherein L¹ is (C₁-C₂)alkylene.

11. The compound of claim 10 wherein R¹ is aryl optionally substituted with one or two alkoxy, nitro, or N(R^{x})(R^{Y}) groups.

12. The compound of claim 9 wherein R² is H.

13. The compound of claim 9 wherein L¹ is a direct bond and R¹ and R² together with the nitrogen attached to R² form a heterocycle group.

14. The compound of claim 13 wherein the heterocycle is a morpholino or piperizino group.

15. The compound of claim 9 wherein R⁴ is H.

16. The compound of claim 9 wherein R³ is a mono- or bi-cyclic heteroaryl group substituted with at least one quaternary ammonium group.

17. The compound of claim 9 wherein R³ is a mono- or bi-cyclic heteroaryl group comprising a nitrogen atom wherein the nitrogen atom is substituted with an optionally substituted alkyl or acyl group.

18. The compound of claim 17 wherein the heteroaryl is an optionally substituted indolyl group.

19. The compound of claim 18 wherein the indolyl group is attached to formula (I) at the indole 5-position.

20. The compound of claim 19 wherein the indolyl group is substituted at its 3-position.

21. The compound of claims 18 wherein the indolyl group is substituted with a N(R^{x})(R^{Y})alkyl- or N⁺(R^{X})(R^{Y})(R^{Z})alkyl-group.

22. The compound claim 21 wherein the N(R^{x})(R^{Y})alkyl-group is dimethylaminomethyl-, dimethylaminoethyl-, or dimethylaminopropyl-.

23. The compound of claim 21 wherein the N⁺(R^{X})(R^{Y})(R^{Z})alkyl-group is dimethyl((C₁-C₁₀)alkyl)ammonium ethyl-.

24. The compound of claim 23 wherein the N⁺(R^{X})(R^{Y})(R^{Z})alkyl-group forms an ion pair with a halide.

25. The compound of claim 9 wherein the compound of formula (II) is a compound of formula (III): 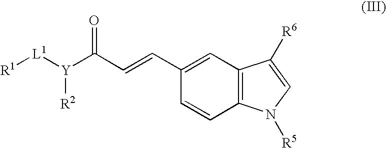
wherein L¹, R¹, and R² are as defined in claim 9;
R⁵ is H, alkyl, aralkyl, or a nitrogen protecting group, wherein alkyl, aralkyl, or the nitrogen protecting group can be optionally substituted with one to five substituents; and
R¹ is an N⁺(R^{X})(R^{Y})(R^{Z})alkyl-group;
or a salt thereof.

26. The compound of claim 25 wherein the compound of formula (I) is N-(2-(5-(3-(4-methoxybenzylamino)-3-oxoprop-1-enyl)-1H-indol-3-yl)ethyl)-N,N-dimethyloctan-1-ammonium iodide (3070); or 6-(3-(4-methoxybenzylamino)-3-oxoprop-1-enyl)-1-methylquinolinium iodide (3061).

27. The compound N-(biphenyl-2-ylmethyl)-N,N-dimethyldodecan-1-aminium halide, wherein halide is fluoride, chloride, bromide, or iodide.

28. The compound N-(4-(dimethylamino)benzyl)-3-(3-(2-(dimethylamino)-ethyl)-1H-indol-5-yl)acrylamide (3043); 3-(3-(2-(dimethylamino)ethyl)-1-methyl-1H-indol-5-yl)-N-(4-methoxybenzyl)acrylamide (3049); N-(2,4-dimethoxybenzyl)-3-(3-(2-(dimethylamino)ethyl)-1H-indol-5-yl)acrylamide (3051); 3-(3-(2-(dimethylamino)ethyl)-1H-indol-5-yl)-N-(4-methoxyphenethyl)acrylamide (3062); 3-(3-(2-(dimethylamino)ethyl)-1H-indol-5-yl)-N-(4-nitrobenzyl)acrylamide (3063); 3-(3-(2-(dimethylamino)ethyl)-1H-indol-5-yl)-1-morpholinoprop-2-en-1-one (3064); 3-(3-((dimethylamino)methyl)-1H-indol-5-yl)-N-(4-methoxybenzyl)-acrylamide (3067); 3-(3-(3-(dimethylamino)propyl)-1H-indol-5-yl)-N-(4-methoxybenzyl)acrylamide (3071); 2-cyano-3-(3-(2-(dimethylamino)ethyl)-1H-indol-5-yl)-N-(4-methoxybenzyl)acrylamide (3044); N-(4-methoxybenzyl)-3-(quinolin-6-yl)acrylamide (3046); 3-(1-benzyl-3-(2-(dimethylamino)ethyl)-1H-indol-5-yl)-N-(4-methoxybenzyl)acrylamide (3055); 3-(1H-indol-5-yl)-N-(4-methoxybenzyl)acrylamide (3056); 3-(3-(2-(dimethylamino)ethyl)-1H-indol-5-yl)-N-(3,4,5-trimethoxybenzyl)acrylamide (3072); 3-(3-(2-(dimethylamino)ethyl)-1H-indol-5-yl)-N-(2,4,6-trimethoxybenzyl)acrylamide (3073); 2-(3-(2-(dimethylamino)ethyl)-1H-indol-5-yloxy)-N-(4-methoxybenzyl)acetamide (3031); 4-methoxybenzyl 3-(3-(2-(dimethylamino)ethyl)-1H-indol-5-yl)acrylate (3045); 3-(3-(2-(dimethylamino)ethyl)-1H-indol-5-yl)-N-hexylacrylamide (3050); N-(4-methoxybenzyl)-2-(quinolin-6-yl)acrylamide (3053); 2-(1H-indol-5-yl)-N-(4-methoxybenzyl)acrylamide (3054); N-(4-methoxybenzyl)-3-(4-methyl-2-oxo-2H-chromen-6-yl)acrylamide (3057); N-(4-methoxybenzyl)-2-(4-methyl-2-oxo-2H-chromen-6-yl)acrylamide (3058); or 10-bromo-2-cyano-N-(4-methoxybenzyl)dec-2-enamide (3059); or a (C₁-C₂₀)alkyl halide salt thereof.

29. An assay kit comprising:
a coelenterazine or a derivative thereof or a luciferase which employs the coelenterazine or derivative thereof as a substrate;
a suitable first container, coelenterazine or derivative thereof or the luciferase disposed therein;
a composition comprising a compound of formula (I): 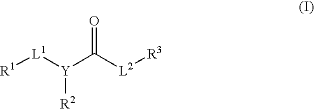
wherein
Y is N or O;
L¹ is (C₁-C₆)alkylene or a direct bond;
R¹ is alkyl, aryl, heteroaryl, or heterocycle;
R² is H, (C₁-C₆)alkyl, or absent;
or L¹ is a direct bond and R¹ and R² together with the nitrogen attached to R² form a heteroaryl or heterocycle group;
L¹ is optionally unsaturated straight chain or branched (C₁-C₆)alkylene or (C₁-C₆)alkylene-O—;
R³ is alkyl, aryl, heteroaryl, heterocycle;
wherein any alkyl, alkylene, aryl, heteroaryl, or heterocycle is optionally substituted with one to five alkyl, alkenyl, alkoxy, alkanoyl, alkanoyloxy, alkoxycarbonyl, cycloalkyl, cyano, nitro, halo, hydroxy, mercapto, oxo, —SOₙR⁴, N(R^{X})(R^{Y}), N(R^{X})(R^{Y})alkyl, N⁺(R^{X})(R^{Y})(R^{Z}), or N⁺(R^{X})(R^{Y})(R^{Z})alkyl groups wherein R^{X}, R^{Y}, and R^{Z} are each independently H, alkyl, aryl, heteroaryl, or heterocycle; and each substituent is optionally substituted with one to three R³ groups; and any nitrogen atom of a nitrogen heterocycle is optionally substituted with an optionally substituted alkyl or acyl group, or with a nitrogen protecting group;
n is 0, 1, 2, or 3; and
R⁴ is H, alkyl, or aryl;
or a salt thereof;
or a compound of formula (IV): 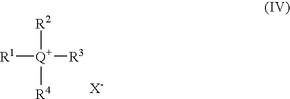
wherein
Q is N or P;
R¹ and R² are each independently alkyl, cycloalkyl, aryl, heteroaryl, heterocycle, arylalkyl;
R³ and R⁴ are each independently (C₁-C₆)alkyl; and
X is an organic or inorganic counterion;
or a combination thereof; and
a suitable second container, the composition disposed therein.

30. A dual reporter enzyme-mediated luminescence reaction assay kit comprising:
a first enzyme substrate for a first enzyme-mediated luminescence reaction;
a suitable first container, the first substrate disposed therein;
a quench-and-activate composition comprising a compound of formula (I): 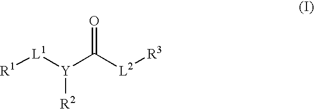
wherein
Y is N or O;
L¹ is (C₁-C₆)alkylene or a direct bond;
R² is alkyl, aryl, heteroaryl, or heterocycle;
R² is H, (C₁-C₆)alkyl, or absent;
or L¹ is a direct bond and R¹ and R² together with the nitrogen attached to R² form a heteroaryl or heterocycle group;
L² is optionally unsaturated straight chain or branched (C₁-C₆)alkylene or (C₁-C₆)alkylene-O—;
R³ is alkyl, aryl, heteroaryl, heterocycle;
wherein any alkyl, alkylene, aryl, heteroaryl, or heterocycle is optionally substituted with one to five alkyl, alkenyl, alkoxy, alkanoyl, alkanoyloxy, alkoxycarbonyl, cycloalkyl, cyano, nitro, halo, hydroxy, mercapto, oxo, —SOₙR⁴, N(R^{X})(R^{Y}), N(R^{X})(R^{Y})alkyl, N⁺(R^{X})(R^{Y})(R^{Z}), or N⁺(R^{X})(R^{Y})(R^{Z})alkyl groups wherein R^{X}, R^{Y}, and R^{Z} are each independently H, alkyl, aryl, heteroaryl, or heterocycle; and each substituent is optionally substituted with one to three R³ groups; and any nitrogen atom of a nitrogen heterocycle is optionally substituted with an optionally substituted alkyl or acyl group, or with a nitrogen protecting group;
n is 0, 1, 2, or 3; and
R⁴ is H, alkyl, or aryl;
or a salt thereof;
or a compound of formula (IV): 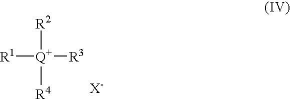
wherein
Q is N or P;
R¹ and R² are each independently alkyl, cycloalkyl, aryl, heteroaryl, heterocycle, arylalkyl;
R³ and R⁴ are each independently (C₁-C₆)alkyl; and
X is an organic or inorganic counterion; or a combination thereof, and a second and distinct functional enzyme substrate for a second and distinct enzyme-mediated luminescence reaction; and
a suitable second container, the quench-and-activate composition disposed therein.

31. The kit of claim 30 wherein one of the substrates is for a beetle luciferase substrate.

32. A kit comprising:
a quench-and-activate composition comprising a compound of formula (I): 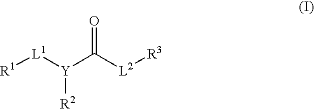
wherein
Y is N or O;
L¹ is (C₁-C₆)alkylene or a direct bond;
R¹ is alkyl, aryl, heteroaryl, or heterocycle;
R² is H, (C₁-C₆)alkyl, or absent;
or L¹ is a direct bond and R¹ and R² together with the nitrogen attached to R² form a heteroaryl or heterocycle group;
L² is optionally unsaturated straight chain or branched (C₁-C₆)alkylene or (C₁-C₆)alkylene-O—;
R³ is alkyl, aryl, heteroaryl, heterocycle;
wherein any alkyl, alkylene, aryl, heteroaryl, or heterocycle is optionally substituted with one to five alkyl, alkenyl, alkoxy, alkanoyl, alkanoyloxy, alkoxycarbonyl, cycloalkyl, cyano, nitro, halo, hydroxy, mercapto, oxo, —SOₙR⁴, N(R^{X})(R^{Y}), N(R^{X})(R^{Y})alkyl, N⁺(R^{X})(R^{Y})(R^{Z}), or N⁺(R^{X})(R^{Y})(R^{Z})alkyl groups wherein R^{X}, R^{Y}, and R^{Z} are each independently H, alkyl, aryl, heteroaryl, or heterocycle; and each substituent is optionally substituted with one to three R³ groups; and any nitrogen atom of a nitrogen heterocycle is optionally substituted with an optionally substituted alkyl or acyl group, or with a nitrogen protecting group;
n is 0, 1, 2, or 3; and
R⁴ is H, alkyl, or aryl;
or a salt thereof;
or a compound of formula (IV): 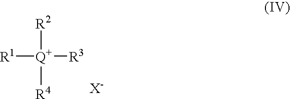
wherein
Q is N or P;
R¹ and R² are each independently alkyl, cycloalkyl, aryl, heteroaryl, heterocycle, arylalkyl;
R³ and R⁴ are each independently (C₁-C₆)alkyl; and
X is an organic or inorganic counterion;
or a combination thereof;
a suitable container, the quench-and-activate composition disposed therein.

33. The kit of claim 32 wherein the composition further comprises reagents for a beetle luciferase-mediated luminescence reaction.

34. A composition comprising a compound of formula (I): 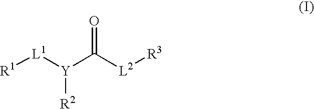
wherein
Y is N or O;
L¹ is (C₁-C₆)alkylene or a direct bond;
R¹ is alkyl, aryl, heteroaryl, or heterocycle;
R is H, (C₁-C₆)alkyl, or absent;
or L¹ is a direct bond and R¹ and R² together with the nitrogen attached to R² form a heteroaryl or heterocycle group;
L² is optionally unsaturated straight chain or branched (C₁-C₆)alkylene or (C₁-C₆)alkylene-O—;
R³ is alkyl, aryl, heteroaryl, heterocycle;
wherein any alkyl, alkylene, aryl, heteroaryl, or heterocycle is optionally substituted with one to five alkyl, alkenyl, alkoxy, alkanoyl, alkanoyloxy, alkoxycarbonyl, cycloalkyl, cyano, nitro, halo, hydroxy, mercapto, oxo, —SOₙR⁴, N(R^{X})(R^{Y}), N(R^{X})(R^{Y})alkyl, N⁺(R^{X})(R^{Y})(R^{Z}), or N⁺(R^{X})(R^{Y})(R^{Z})alkyl groups wherein R^{X}, R^{Y}, and R^{Z} are each independently H, alkyl, aryl, heteroaryl, or heterocycle; and each substituent is optionally substituted with one to three R³ groups; and any nitrogen atom of a nitrogen heterocycle is optionally substituted with an optionally substituted alkyl or acyl group, or with a nitrogen protecting group;
n is 0, 1, 2, or 3; and
R⁴ is H, alkyl, or aryl;
or a salt thereof;
or a compound of formula (I) and a compound of formula (IV): 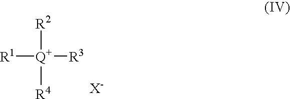
wherein
Q is N or P;
R¹ and R² are each independently alkyl, cycloalkyl, aryl, heteroaryl, heterocycle, arylalkyl;
R³ and R⁴ are each independently (C₁-C₆)alkyl; and
X is an organic or inorganic counterion; and
a suitable solvent.

35. The composition of claim 34 further comprising a sequestering agent.

36. The composition of claim 34 further comprising a yellow colored compound.

37. The composition of claim 34 further comprising a reducing agent.

38. The composition of claim 34 further comprising a cell lysing agent.

39. A method of assaying a luciferase-mediated luminescence reaction comprising:
detecting or determining luminescence energy produced by at least one first enzyme-mediated luminescence reaction in a reaction mixture, wherein the reaction mixture comprises a compound of formula (I): 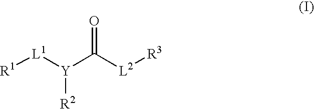
wherein
Y is N or O;
L¹ is (C₁-C₆)alkylene or a direct bond;
R¹ is alkyl, aryl, heteroaryl, or heterocycle;
R² is H, (C₁-C₆)alkyl, or absent;
or L¹ is a direct bond and R¹ and R² together with the nitrogen attached to R² form a heteroaryl or heterocycle group;
L² is optionally unsaturated straight chain or branched (C₁-C₆)alkylene or (C₁-C₆)alkylene-O—;
R³ is alkyl, aryl, heteroaryl, heterocycle;
wherein any alkyl, alkylene, aryl, heteroaryl, or heterocycle is optionally substituted with one to five alkyl, alkenyl, alkoxy, alkanoyl, alkanoyloxy, alkoxycarbonyl, cycloalkyl, cyano, nitro, halo, hydroxy, mercapto, oxo, —SOₙR⁴, N(R^{X})(R^{Y}), N(R^{X})(R^{Y})alkyl, N⁺(R^{X})(R^{Y})(R^{Z}), or N⁺(R^{X})(R^{Y})(R^{Z})alkyl groups wherein R^{X}, R^{Y}, and R^{Z} are each independently H, alkyl, aryl, heteroaryl, or heterocycle; and each substituent is optionally substituted with one to three R³ groups; and any nitrogen atom of a nitrogen heterocycle is optionally substituted with an optionally substituted alkyl or acyl group, or with a nitrogen protecting group;
n is 0, 1, 2, or 3; and
R⁴ is H, alkyl, or aryl;
or a salt thereof;
or a compound of formula (I) and a compound of (IV): 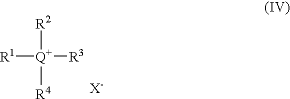
wherein
Q is N or P;
R¹ and R² are each independently alkyl, cycloalkyl, aryl, heteroaryl, heterocycle, arylalkyl;
R³ and R⁴ are each independently (C₁-C₆)alkyl; and
X is an organic or inorganic counterion.

40. A method of assaying an enzyme-mediated luminescence reaction comprising:
(a) detecting or determining luminescence energy produced by a luciferase and a coelenterazine or a derivative thereof in a luciferase-mediated luminescence reaction; and
(b) quenching photon emission from the luminescence reaction by introducing a composition comprising a compound of formula (I): 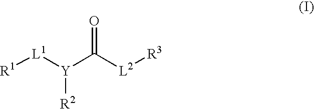
wherein
Y is N or O;
L¹ is (C₁-C₆)alkylene or a direct bond;
R¹ is alkyl, aryl, heteroaryl, or heterocycle;
R is H, (C₁-C₆)alkyl, or absent;
or L¹ is a direct bond and R¹ and R² together with the nitrogen attached to R² form a heteroaryl or heterocycle group;
L² is optionally unsaturated straight chain or branched (C₁-C₆)alkylene or (C₁-C₆)alkylene-O—;
R³ is alkyl, aryl, heteroaryl, heterocycle;
wherein any alkyl, alkylene, aryl, heteroaryl, or heterocycle is optionally substituted with one to five alkyl, alkenyl, alkoxy, alkanoyl, alkanoyloxy, alkoxycarbonyl, cycloalkyl, cyano, nitro, halo, hydroxy, mercapto, oxo, —SOₙR⁴, N(R^{x})(R^{Y}), N(R^{x})(R^{Y})alkyl, N⁺(R^{X})(R^{Y})(R^{Z}), or N⁺(R^{X})(R^{Y})(R^{Z})alkyl groups wherein R^{X}, R^{Y}, and R^{Z} are each independently H, alkyl, aryl, heteroaryl, or heterocycle; and each substituent is optionally substituted with one to three R³ groups; and a nitrogen atom of a nitrogen heterocycle is optionally protected with a nitrogen protecting group;
n is 0, 1, 2, or 3; and
R⁴ is H, alkyl, or aryl;
or a salt thereof;
or a compound of formula (IV): 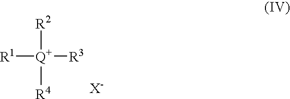
wherein
Q is N or P;
R¹ and R² are each independently alkyl, cycloalkyl, aryl, heteroaryl, heterocycle, arylalkyl;
R³ and R⁴ are each independently (C₁-C₆)alkyl; and
X is an organic or inorganic counterion;
or a combination thereof, to the luminescence reaction.

41. The method of claim 40 wherein the composition is capable of selectively quenching the luminescence reaction and initiating a second enzyme-mediated luminescence reaction distinct from the luciferase-mediated luminescence reaction in (a); and
(c) detecting or determining luminescence energy produced by the second enzyme-mediated luminescence reaction.

42. The method of claim 40 further comprising:
(c) introducing a composition capable of initiating a second enzyme-mediated luminescence reaction distinct from the luciferase-mediated luminescence reaction in (a); and
(d) detecting or determining luminescence energy produced by the second enzyme-mediated luminescence reaction.
